SEQUENCE LISTING (8 sequences)

SEQ ID NO: 1 - protein, 20 aa, Artificial Sequence
  Synthetic construct; BIN3 forward primer
TTGCAGCCTGTGTGTCTAAG

SEQ ID NO: 2 - protein, 20 aa, Artificial Sequence
  Synthetic construct; BIN3 reverse primer
CTCCAGGAAGTGACGTAAGC

SEQ ID NO: 3 - protein, 18 aa, Artificial Sequence
  Synthetic construct; EMP1 forward primer
AAAGTGGATACAGAGACA

SEQ ID NO: 4 - protein, 18 aa, Artificial Sequence
  Synthetic construct; EMP1 reverse primer
GTGAAAAACATCTGGCCA

SEQ ID NO: 5 - protein, 22 aa, Artificial Sequence
  Synthetic construct; BIN3 primer
CCCAGGGACCTCTCTCTAATCA

SEQ ID NO: 6 - protein, 20 aa, Artificial Sequence
  Synthetic construct; BIN3 primer
GCTACAGGCTTGTCACTCGG

SEQ ID NO: 7 - protein, 20 aa, Artificial Sequence
  Synthetic construct; GAPDH primer
GTGAAGGTCGGAGTCAACGG

SEQ ID NO: 8 - protein, 22 aa, Artificial Sequence
  Synthetic construct; GAPDH primer
TGATGACAAGCTTCCCGTTCTC

(19) United States
(12) Patent Application Publication
(10) Pub. No.: US 2023/0075368 A1
(43) Pub. Date: Mar. 9, 2023

(54) IDENTIFICATION OF AN EGFR-BIN3 PATHWAY THAT ACTIVELY SUPPRESSES INVASION AND REDUCES TUMOR SIZE IN GLIOBLASTOMA

(71) Applicant: The United States Govemment as represented by the Department of Veterans Affairs, Washington, DC (US)

(72) Inventor: Amyn Habib, Dallas, TX (US)

(21) Appl. No.: 17/800,210

(22) PCT Filed: Feb. 19, 2021
(86) PCT No.: PCT/US21/18716

§ 371 Date: Aug. 16, 2022

Related U.S. Application Data
(60) Provisional application No. 62/978,776, filed on Feb. 19, 2020.
(60) Provisional application No. 63/027,852, filed on May 20, 2020.

(51) Int. Cl. A61K 31/519; A61K 31/517; A61K 31/495; A61K 45/06; A61K 31/506; A61K 31/137; A61K 31/437; A61K 38/18; A61K 38/17; A61P 35/00

(52) U.S. Cl. CPC A61K 31/519; A61K 31/517; A61K 31/495; A61K 45/06; A61K 31/506; A61K 31/137; A61K 31/437; A61K 38/1808; A61K 38/1841; A61K 38/1796; A61P 35/00

(57) ABSTRACT

The present disclosure is concerned with the use of agents that modulate bridging integrator 3 (BIN3) signaling and/or janus kinase 3 (JAK3) signaling for treating various gliomas such as, for example, glioblastoma. The present disclosure is also concerned with the use of agents that increase levels of EGFR ligand (e.g., tofacitinib, EGFR ligands) for treating gliomas, and, in particular, for treating EGFR amplified gliomas. This abstract is intended as a scanning tool for purposes of searching in the particular art and is not intended to be limiting of the present invention.

### CROSS-REFERENCE TO RELATED APPLICATIONS

This application claims the benefit of U.S. Application No. 62/978,776, filed on Feb. 19, 2020, and U.S. Application No. 63/027,852, filed on May 20, 2020, the contents of which are hereby incorporated by reference in their entirety.

### REFERENCE TO SEQUENCE LISTING

The Sequence Listing submitted Feb. 18, 2021 as a text file named “37759 0301P1 ST25.txt,” created on Feb. 4, 2021, and having a size of 2,798 bytes is hereby incorporated by reference pursuant to 37 C.F.R. § 1.52(e)(5).

### BACKGROUND

The invasive property of cancer cells is considered a hallmark of cancer and plays a particularly important role in glioblastoma (GBM), the most common primary malignant brain tumor in adults. GBM is an untreatable and devastating disease largely because of its highly invasive nature, rendering complete surgical removal impossible (Altieri et al. (2015) Surg. Technol. Int 27: 297-302; Armento et al. (2017) Molecular Mechanisms of Glioma Cell Motility, In Glioblastoma (De Vleeschouwer, S., Ed.), Brisbane (AU)). It has been proposed that glioma invasion and proliferation are spatiotemporally distinct and may be mutually exclusive, the “go or grow hypothesis” (Xie et al. (2014) Neuro. Oncol. 16: 1575-1584; Venere et al. (2015) Sci. Transl. Med. 7: 304ra143; Hatzikirou et al. (2012) Math Med. Biol. 29: 49-65; Horing et al. (2012) Acta neuropathological 124: 83-97; Newman et al. (2017) Nat. Commun. 8: 1913; Dhruv et al. (2013) PLoS One 8: e72134; Gao et al. (2005) Proc. Nat. Acad. Sci. U.S.A. 102: 10528-10533). Thus, under adverse environmental conditions such as hypoxia, mechanisms are triggered that make the glioma cell “go” or invade, whereas in favorable environments glioma cells “grow” or proliferate. A number of environmental and intracellular signaling pathways have been implicated in the invasion vs. proliferation decision including cytoskeletal dynamics, cell volume, and the extracellular composition (Hatzikirou et al. (2012) Math Med. Biol. 29: 49-65; Giese et al. (1996) Int. J. Cancer 67: 275-282). It has been reported that the pentose phosphate pathway (PPP) is used mainly during invasion while glycolysis is used as the energy source during invasion (Kathagen-Buhmann et al. (2016) Neuro. Oncol. 18: 1219-1229). Increased c-Myc activity was reported in proliferating cells while increased NF-κB activation was found in invasive glioma cells (Dhruv et al. 2913) PLoS One 8: e72134). The molecular motor kinesin KIF11 has been reported to play a role in both proliferation and invasion (Venere et al. (2015) Sci. Transl. Med. 7: 304ra143). Previous studies have also reported a role for EGFR signaling in promoting multiple aspects of the malignant phenotype including proliferation and invasion in GBM (Newman et al. (2017) Nat. Commun. 8: 1913; Talasila et al. (2013) Acta neuropathological 125: 683-698; Roos et al. (2018) Mol. Cancer Res. 16: 1185-1195; Ding et al. (2018) Mol. Cancer Res. 16: 322-332; Huang et al. (2009) Sci. Signal 2: re6; Hatanpaa et al. (2010) Neoplasia 12: 675-684), although much remains to be learned about how the EGFR differentially regulates these processes.

Bridging integrator 3 (BIN3) is a member of the Bin-Ampiphysin-Rvs (BAR) domain family of proteins that regulate membrane and actin dynamics (Habermann, B. (2004) EMBO reports 5: 250-255). BIN3 is ubiquitously expressed and conserved throughout evolution. BAR domain proteins also regulate Rho GTPases that are involved in GBM invasion (de Kreuk and Hordijik (2012) Small GTPases 3: 45-52; Simionescu-Bankston et al. (2013) Dev. Biol. 382: 160-171; Rotin et al. (2013) Front Oncol. 3: 241). BIN3 maps to chromosome 8p21.3 a tumor suppressor region that is often deleted in non-Hodgkin's lymphoma and other epithelial cancers (Binrbaum et al. (2003) The lancet oncology 4: 639-642; Rubio-Moscardo et al. (2005) Blood 10: 3214-3222; Chang et al. (2007) Cancer Res. 67: 4098-4103; Ye et al. (2007) Cancer Genet. Cytogenet. 176: 100-106). BIN3 deletion in mice results in increased susceptibility to lymphoma (Ramalingam et al. (2008) Cancer Res. 68: 1683-1690). Another BAR family member BIN1, functions as a tumor suppressor gene in multiple cancer types (Prendergast et al. (2009) Biochim Biophys Acta 1795: 25-36).

EGFR gene amplification is found in the classical subtype of GBM (Verhakk et al. (2010) Cancer Cell 17: 98-110), and is detected in 40-50% of GBMs (Hatanpaa et al. (2010) Neoplasia 12: 675-684; Frederick et al. (2000) Cancer Res. 60: 1388-1387), resulting in EGFR overexpression. However, expression of EGFR is also detected without gene amplification and has been detected in up to 81% of GBM, transcending the molecular subgroups of GBMs (Hatanpaa et al. (2010) Neoplasia 12: 675-684; Fan et al. (2013) Cancer Cell 24: 438-449). Both EGFR wild type (EGFRwt) and constitutively active pro-invasive mutants such as EGFRvIII are expressed (Roos et al. (2018) Mol. Cancer Res. 16: 1185-1195; Huang et al. (2009) Sci. Signal 2: re6; Hatanpaa et al. (2010) Neoplasia 12: 675-684; Batra et al. (1995) Cell Growth Differ. 6: 1251-1259). EGFRwt also plays an oncogenic role in GBM and is a transforming oncogene (Talasila et al. (2013) Acta neuropathological 125: 683-698; Acquaviva et al. (2011) Cancer Res. 71: 7198-7206; Di Fiore et al. (1987) Cell 51: 1063-1070; Velu et al. (1987) Science 238: 1408-1410). EGFRwt may be activated by ligand binding or signal constitutively when overexpressed in cancer (Acquaviva et al. (2011) Cancer Res. 71: 7198-7206; Nishikawa et al. (1994) Proc. Natl. Acad. Sci. U.S.A. 91: 7727-7731; Chakraborty et al. (2014) Nat. Commun. 5: 5811; Wong et al. (1992) Proc. Natl. Acad. Sci. U.S.A. 89: 2965-2969; Guo et al. (2015) Cancer Res. 75: 3436-3441; Endres et al. (2013) Cell 152: 543-556). There is also evidence that EGFRwt and EGFRvIII are expressed in the same tumor and activate each other (Horing et al. (2012) Acta neuropathological 124: 83-97; Newman et al. (2017) Nat. Commun. 8: 1913; Huang et al. (2009) Sci. Signal 2: re6; Hatanpaa et al. (2010) Neoplasia 12: 675-684; Habermann, B. (2004) EMBO reports 5: 250-255; de Kreuk et al. (2012) Small GTPases 3: 45-52). Cellular stress may also result in a ligand-independent EGFR signaling (Tan et al. (2016) Trends Cell Biol. 26: 352-366). Constitutive signaling is defined here as signaling triggered by EGFR expression in GBM leading to spontaneous dimerization and downstream signaling in the absence of EGFR ligand (Guo et al. (2015) Cancer Res. 75: 3436-3441; Endres et al. (2013) Cell 152: 543-556). It has been reported previously reported that EGFRwt signaling is bimodal and demonstrated that constitutive and ligand-induced EGFR signaling trigger distinct and non-overlapping signaling pathways leading to distinct biological responses (Chakraborty et al. (2014) Nat. Commun. 5: 5811; Guo et al. (2015) Cancer Res. 75: 3436-3441). Despite these advances, there is still much to be learned regarding the role of a ligand-activated EGFR switch in regulating the proliferation and invasion decision, and the identification of EGFR driven pathways that regulate invasion and tumor size in GBM. Accordingly, there remains a need for compositions and methods for treating gliomas, and, in particular, malignant gliomas. These needs and others are met by the present invention.

### SUMMARY

In accordance with the purpose(s) of the invention, as embodied and broadly described herein, the invention, in one aspect, relates to compounds and compositions for use in the prevention and treatment of gliomas such as, for example, malignant gliomas.

Thus, disclosed are methods for treating a subject for glioma, the method comprising administering to the subject an effective amount of an agent that modulates bridging integrator 3 (BIN3) signaling, or a pharmaceutically acceptable salt thereof.

Also disclosed are methods for treating a subject for glioma, the method comprising administering to the subject an effective amount of an agent that modulates JAK3 signaling, or a pharmaceutically acceptable salt thereof.

Also disclosed are methods for treating a glioma in a subject in need thereof, the method comprising administering to the subject an agent that increases EGFR ligand.

Also disclosed are methods for treating an EGFR amplified glioma in a subject in need thereof, the method comprising administering to the subject tofacitinib and an EGFR ligand, wherein at least one of tofacitinib and the EGFR ligand is administered in an effective amount.

Also disclosed are kits comprising an agent that modulates BIN3 signaling, or a pharmaceutically acceptable salt thereof, and one or more of: (a) an agent associated with the treatment of cancer; (b) an agent associated with the treatment of inflammation; (c) instructions for administering the agent that modulates BIN3 signaling in connection with treating glioma; and (d) instructions for treating glioma.

Also disclosed are kits comprising an agent that modulates JAK3 signaling, or a pharmaceutically acceptable salt thereof, and one or more of: (a) an agent associated with the treatment of cancer; (b) an agent associated with the treatment of inflammation; (b) instructions for administering the agent that modulates JAK3 signaling in connection with treating glioma; and (c) instructions for treating glioma.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIGS. 1A-D show representative images illustrating that tofacitinib inhibits the growth of intracranial tumors in mice. Specifically, FIG. 1A and FIG. 1C shows that treatment with tofacitinib prolong survival in a GBM12 or GBM6 (two different patient derived xenografts) orthotopic model (n=8). One week after intracranial injection, mice were divided into 2 groups and treated with control vehicle of tofacitinib (40 mg/kg/day for 3 weeks) (n=8). Kaplan-Meier survival curves were calculated using GraphPad Prism 7. Statistical significance verified by the log rank test. *P<0.05. FIG. 1B and FIG. 1D show that tofacitinib treated tumors (T) do not infiltrate the surrounding normal tissue (NT), whereas vehicle treated tumor do infiltrate into surrounding normal tissue.

FIGS. 2A-K and FIG. 3A-N show representative data illustrating that EGFR signaling activity effects invasion of PDXs.

FIGS. 4A-D show representative data analyzing glioma cell migration by in vitro scratch assays.

FIG. 5A and FIG. 5B show representative data from the BrdU incorporation assay in neuriospheres.

FIGS. 6A-E show representative data illustrating that EGF inhibits EGFR overexpression induced cell invasion.

FIGS. 7A-O show representative data illustrating that EGF-mediated BIN3 inhibits invasiveness.

FIGS. 8A-C show repsentative data illustrating that EGF induces EGR1 activity and enrichment on the BIN3 promoter.

FIGS. 9A-S show representative data illustrating that BIN3 reduces invasion by interacting with DOCK7.

FIGS. 10A-I show representative data illustrating that siRNA-mediated knockdown of CDC42 or RhoA reduced invasiveness.

FIGS. 11A-E show representative data illustrating that HGF induces invasion in GBM12 and GBM6.

FIGS. 12A-O show representative data illustrating that ligand induced EGFR signaling inhibits invasion of PDXs.

FIGS. 13A-H show representative data illustrating that TGFα overexpression prolongs survival, reduces invasiveness, and increases proliferation in orthotopic glioblastoma mouse model.

FIGS. 14A and FIG. 14B show representative data illustrating that EGF reduces cell migration speed in vivo detected by intravital microscopy.

FIGS. 15A-G show representative data illustrating that EGF overexpression prolongs survival, reduces invasiveness, and increases proliferation in an orthotopic glioblastoma mouse model.

FIGS. 16A-J show representative data illustrating that BIN3 overexpression inhibits invasiveness.

FIG. 17A and FIG. 17B show representative data illustrating that BIN3 overexpression inhibits invasion of GBM22.

FIGS. 18A-P and FIG. 19A-F show representative data illustrating that tofacitinib inhibits invasion of PDXs by upregulation of BIN3.

FIGS. 20A-M show representative data illustrating that EGR1 is required for both EGF- and tofacitinib-induced BIN3 expression.

FIGS. 21A-N show representative data illustrating that tofacitinib prolongs survival of mice bearing orthotopic glioblastoma tumors.

FIGS. 22A-C show representative data illustrating that tofacitinib inhibits invasion of GBM44 with HB-EGF knockdown.

FIGS. 23A-P show representative data illustrating that EGFR induced EMP1 overexpression drives invasion and is mediated by Nanog.

FIGS. 24A-C show representative data illustrating the results of a Cignal 45 pathway reporter assay and a schematic diagram of EMP-1 promoter.

FIGS. 25A-P show representative data illustrating that EGF and tofacitinib result in reduced migration and enhanced proliferation in single cell analysis.

FIGS. 26A-N show representative data illustrating that EGF and tofacitinib result in reduced cell migration velocity and enhanced cell proliferation.

FIGS. 27A-L show representative data illustrating EGFR ligands and BIN3 expression in human glioblastoma.

FIGS. 28A-C show representative data illustrating BIN3 expression in human glioblastoma.

FIGS. 29A-C show a representative survival and correlation analysis according to BIN3, HB-EFG, and EGFR expression.

FIGS. 30A-C show a representative overall survival analysis according to BIN3 expression in three cancer types.

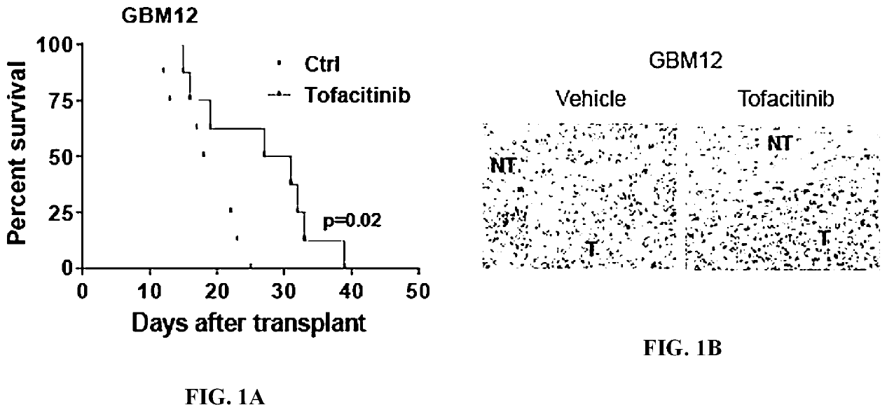

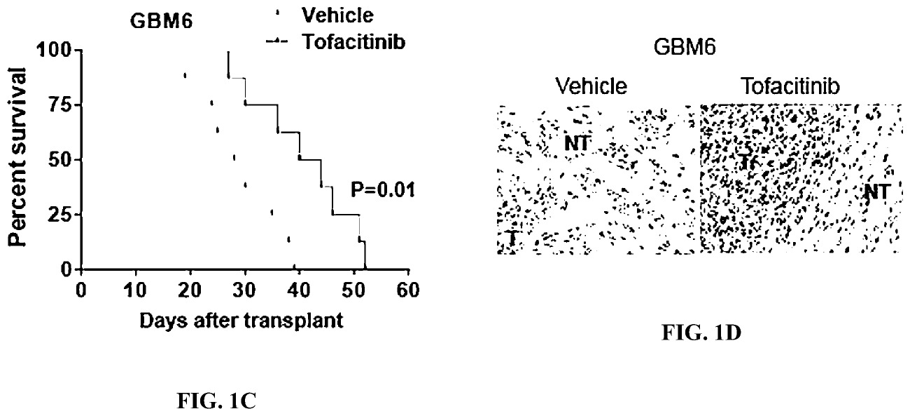

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “EGFR amplified,” when used in reference to a cancer such as, for example, a glioma, means a cancer that carries an increased number of copies of the EGFR gene compared to normal cells. Thus, for example, an EGFR amplified cancer can carry at least 5% more copies, at least 10% more copies, at least 15% more copies, at least 20% more copies, at least 25% more copies, at least 30% more copies, at least 35% more copies, at least 40% more copies, at least 45% more copies, at least 50% more copies, at least 55% more copies, at least 60% more copies, at least 65% more copies, at least 70% more copies, at least 75% more copies, at least 80% more copies, at least 85% more copies, at least 90% more copies, or at least 95% more copies relative to normal cells.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. METHODS FOR TREATING GLIOMA USING AN AGENT THAT MODULATES BIN3 SIGNALING

In one aspect, disclosed are methods for treating a subject for glioma, the method comprising administering to the subject an effective amount of an agent that modulates bridging integrator 3 (BIN3) signaling, or a pharmaceutically acceptable salt thereof.

In one aspect, disclosed are methods for treating a malignant glioma in a patient in need thereof, said method comprising administering to said patient an effective amount of tofacitinib and temozolomide. In a further aspect, the effective amount is an individually effective amount of tofacitinib and/or temozolomide. In a still further aspect, the effective amount is an individually effective amount of tofacitinib. In yet a further aspect, the effective amount is an individually effective amount of temozolomide. In an even further aspect, the effective amount is a combinatorically effective amount of tofacitinib and temozolomide.

In various aspects, the agent that modulates BIN3 signaling is a BIN3 activator. In various further aspects, the agent activates BIN3 with an EC₅₀ of less than about 200 nM, less than about 180 nM, less than about 160 nM, less than about 140 nM, less than about 120 nM, less than about 100 nM, less than about 80 nM, less than about 40 nM, or less than about 20 nM. In a further aspect, the agent activates BIN3 with an EC₅₀ of less than about 100 nM.

In various aspects, the BIN3 activator inhibits janus kinase 3 (JAK3) signaling. In various further aspects, the BIN3 activator inhibits JAK3 signaling with an IC₅₀ of less than about 10 nM, less than about 8 nM, less than about 6 nM, less than about 4 nM, less than about 2 nM, less than about 1 nM, less than about 0.8 nM, or less than about 0.6 nM. In a further aspect, the BIN3 activator inhibits JAK3 signaling with an IC₅₀ of less than about 1 nM.

In various aspects, the BIN3 activator inhibits janus kinase 1 (JAK1) signaling. In various further aspects, the BIN3 activator inhibits JAK1 signaling with an IC₅₀ of less than about 150 nM, less than about 140 nM, less than about 130 nM, less than about 120 nM, less than about 110 nM, or less than about 100 nM. In a further aspect, the BIN3 activator inhibits JAK1 signaling with an IC₅₀ of less than about 130 nM.

In various aspects, the BIN3 activator inhibits JAK1 and JAK3 signaling.

In various aspects, the BIN3 activator does not substantially inhibit janus kinase 2 (JAK2) signaling. For example, the BIN3 activator can inhibit JAK2 signaling with an IC₅₀ of about 20 nM or more, about 30 nM or more, about 40 nM or more, about 50 nM or more, about 60 nM or more, about 70 nM or more, or about 80 nM or more. In a further aspect, the BIN3 activator can inhibit JAK2 signaling with an IC₅₀ of about 20 nM or more. In a further aspect, the BIN3 activator inhibits JAK2 and JAK3 signaling, and inhibits JAK2 with an IC₅₀ that is at least ten times greater, at least fifteen times greater, or at least twenty times greater than the IC₅₀ for JAK3 signaling (i.e., JAK3 is activated at a concentration that is at least about ten times less, at least about fifteen times less, or at least about twenty times less than the concentration needed to activated JAK2). In a still further aspect, the BIN3 activator does not inhibit JAK2. okay

In various aspects, the agent that modulates BIN3 signaling is tofacitinib.

In various aspects, the method further comprises administering to the subject an effective amount of an agent that modulates epidermal growth factor receptor (EGFR) signaling, or a pharmaceutically acceptable salt thereof. In various further aspects, the agent that modulates EGFR signaling is an EGFR inhibitor. In a further aspect, the EGFR inhibitor is a tyrosine kinase inhibitor. Examples of tyrosine kinase inhibitors include, but are not limited to, erlotinib. In a still further aspect, the EGFR inhibitor is a monoclonal antibody.

In various aspects, the EGFR inhibitor is selected from erlotinib, afatinib, cetuximab, panitumumab, erlotinib HCl, gefitinib, lapatinib, neratinib, lifirafenib, HER2-inhibitor-1, nazartinib, naquotinib, canertinib, AG-490, CP-724714, Dacomitinib, WZ4002, Sapitinib, CUDC-101, AG-1478, PD153035 HCl, pelitinib, AC480, AEE788, AP261 13-analog, OSI-420, WZ3146, WZ8040, AST-1306, rociletinib, genisten, varlitinib, icotinib, TAK-285, WHI-P154, daphnetin, PD168393, tyrphostin9, CNX-2006, AG-18, AZ5104, osimertinib, CL-387785, olmutinib, AZD3759, poziotinib, vandetanib, and necitumumab.

In various aspects, the agent that modulates BIN3 signaling is a BIN3 activator and wherein the agent that modulates EGFR signaling is an EGFR inhibitor.

In various aspects, the agent that modulates BIN3 signaling and the agent that modulates EGFR signaling are co-formulated. In various further aspects, the agent that modulates BIN3 signaling and the agent that modulates EGFR signaling are co-packaged.

In various aspects, the agent that modulates BIN3 signaling and the agent that modulates EGFR signaling are administered concurrently. In various further aspects, the agent that modulates BIN3 signaling and the agent that modulates EGFR signaling are not administered concurrently.

In various aspects, the method further comprises administering to the subject an effective amount of an agent associated with the treatment of glioma. Examples of agents known for the treatment of glioma include, but are not limited to, tumor treating fields, bevacizumab, and radiation therapy. In a further aspect, the agent associated with the treatment of glioma is temozolomide.

In various aspects, the agent that modulates BIN3 signaling is tofacitinib and wherein the agent associated with the treatment of glioma is temozolomide.

In various aspects, the agent that modulates BIN3 signaling and the agent associated with the treatment of glioma are co-formulated. In various further aspects, the agent that modulates BIN3 signaling and the agent associated with the treatment of glioma are co-packaged.

In various aspects, the agent that modulates BIN3 signaling and the agent associated with the treatment of glioma are administered concurrently. In various further aspects, the agent that modulates BIN3 signaling and the agent associated with the treatment of glioma are not administered concurrently.

In various aspects, the method further comprises administering to the subject an effective amount of an agent associated with the treatment of inflammation, such as, for example, a glucocorticoid. Examples of glucocorticoids include, but are not limited to, beclomethason, betamethasone, budesonide, cortisone, dexamethasone, hydrocortisone, methylprednisolone, prednisolone, prednisone, and triamcinolone.

In various aspects, the agent that modulates BIN3 signaling and the agent associated with the treatment of inflammation are co-formulated. In various further aspects, the agent that modulates BIN3 signaling and the agent associated with the treatment of inflammation are co-packaged.

In various aspects, the agent that modulates BIN3 signaling and the agent associated with the treatment of inflammation are administered concurrently. In various further aspects, the agent that modulates BIN3 signaling and the agent associated with the treatment of inflammation are not administered concurrently.

In various aspects, the method further comprises administering to the subject an effective amount of an EGFR ligand. Examples of EGFR ligands include, but are not limited to, EGF, TGFA, HB-EGF, AR, EREG, BTC, and EPGN. In a further aspect, the EGFR ligand is EGF.

In various aspects, the agent that modulates BIN3 signaling and the EGFR ligand are co-formulated. In various further aspects, the agent that modulates BIN3 signaling and the EGFR ligand are co-packaged.

In various aspects, the agent that modulates BIN3 signaling and the EGFR ligand are administered concurrently. In various further aspects, the agent that modulates BIN3 signaling and the EGFR ligand are not administered concurrently.

In various aspects, the effective amount is a therapeutically effective amount. In a further aspect, the effective amount is a prophylactically effective amount.

In various aspects, the effective amount is a concentration of less than about 10 nM, less than about 9 nM, less than about 8 nM, less than about 7 nM, less than about 6 nM, less than about 5 nM, less than about 4 nM, less than about 3 nM, less than about 2 nM, less than about 1 nM, less than about 0.8 nM, less than about 0.4 nM, or less than about 0.2 nM. In a further aspect, the effective amount is a concentration of less than about 1 nM.

In a further aspect, the subject has been diagnosed with a need for treatment of glioma prior to the administering step. In a still further aspect, the subject is at risk for developing glioma prior to the administering step.

In a further aspect, the subject is a mammal. In a still further aspect, the mammal is a human.

In a further aspect, the method further comprises the step of identifying a subject in need of treatment of glioma.

In various aspects, the glioma is a glioblastoma.

In a further aspect, the glioma expresses EGFR wild type. In a still further aspect, the glioma expresses EGFR mutant. In yet a further aspect, the glioma is resistant to EGFR inhibition.

In a further aspect, the method further comprises the step of administering a therapeutically effective amount of at least one chemotherapeutic agent. In yet a further aspect, the chemotherapeutic agent is selected from an alkylating agent, an antimetabolite agent, an antineoplastic antibiotic agent, a mitotic inhibitor agent, and an mTor inhibitor agent.

In various aspects, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In various aspects, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In various aspects, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In various aspects, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In various aspects, the mTor inhibitor agent is selected from everolimus, siroliumus, and temsirolimus, or a pharmaceutically acceptable salt, hydrate, solvate, or polymorph thereof.

### C. METHODS FOR TREATING GLIOMA USING AN AGENT THAT MODULATES JAK3 SIGNALING

In one aspect, disclosed are methods for treating a subject for glioma, the method comprising administering to the subject an effective amount of an agent that modulates JAK3 signaling, or a pharmaceutically acceptable salt thereof.

In one aspect, disclosed are methods for treating a malignant glioma in a patient in need thereof, said method comprising administering to said patient an effective amount of tofacitinib and temozolomide. In a further aspect, the effective amount is an individually effective amount of tofacitinib and/or temozolomide. In a still further aspect, the effective amount is an individually effective amount of tofacitinib. In yet a further aspect, the effective amount is an individually effective amount of temozolomide. In an even further aspect, the effective amount is a combinatorically effective amount of tofacitinib and temozolomide.

In various aspects, the agent that modulates JAK3 signaling is a JAK3 inhibitor. In various further aspects, the agent inhibits JAK3 signaling with an IC₅₀ of less than about 10 nM, less than about 8 nM, less than about 6 nM, less than about 4 nM, less than about 2 nM, less than about 1 nM, less than about 0.8 nM, or less than about 0.6 nM. In a further aspect, the agent inhibits JAK3 signaling with an IC₅₀ of less than about 1 nM.

In various aspects, the JAK3 inhibitor also inhibits janus kinase 1 (JAK1) signaling. In various further aspects, the agent inhibits JAK1 signaling with an IC₅₀ of less than about 150 nM, less than about 140 nM, less than about 130 nM, less than about 120 nM, less than about 110 nM, or less than about 100 nM. In a further aspect, the agent inhibits JAK1 signaling with an IC₅₀ of less than about 130 nM.

In various aspects, the JAK3 inhibitor does not substantially inhibit janus kinase 2 (JAK2) signaling. For example, the JAK3 inhibitor can also inhibit JAK2 signaling with an IC₅₀ of about 20 nM or more, about 30 nM or more, about 40 nM or more, about 50 nM or more, about 60 nM or more, about 70 nM or more, or about 80 nM or more. In a further aspect, the JAK3 inhibitor can also inhibit JAK2 signaling with an IC₅₀ of about 20 nM or more. In a further aspect, the JAK3 inhibitor inhibits JAK2 with an IC₅₀ that is at least ten times greater, at least fifteen times greater, or at least twenty times greater than the IC₅₀ for JAK3 signaling (i.e., JAK3 is activated at a concentration that is at least about ten times less, at least about fifteen times less, or at least about twenty times less than the concenation needed to activated JAK2). In a still further aspect, the JAK3 inhibitor does not inhibit JAK2 signaling. okay

In various aspects, the JAK3 inhibitor also activates BIN3 signaling. In various further aspects, the JAK3 inhibitor activates BIN3 signaling with an EC₅₀ of about 200 nM or less, about 180 nM or less, about 160 nM or less, about 140 nM or less, about 120 nM or less, about 100 nM or less, about 80 nM or less, or about 60 nM or less. In a further aspect, the JAK3 inhibitor activates BIN3 signaling with an EC₅₀ of about 100 nM or less.

In various aspects, the the JAK3 inhibitor is selected from AZD1480, tofacitinib, tofacitinib citrate, WHI-P154, ZM 39923 HCl, PF-06651600, JANEX-1, FM-381, decernotinib, WHI-P258, and WHI-P97. In a further aspect, the JAK3 inhibitor is tofacitinib.

In various aspects, the method further comprises administering to the subject an effective amount of an agent that modulates epidermal growth factor receptor (EGFR) signaling, or a pharmaceutically acceptable salt thereof. In various further aspects, the agent that modulates EGFR signaling is an EGFR inhibitor. In a further aspect, the EGFR inhibitor is a tyrosine kinase inhibitor. Examples of tyrosine kinase inhibitors include, but are not limited to, erlotinib. In a still further aspect, the EGFR inhibitor is a monoclonal antibody.

In various aspects, the EGFR inhibitor is selected from erlotinib, afatinib, cetuximab, panitumumab, erlotinib HCl, gefitinib, lapatinib, neratinib, lifirafenib, HER2-inhibitor-1, nazartinib, naquotinib, canertinib, AG-490, CP-724714, Dacomitinib, WZ4002, Sapitinib, CUDC-101, AG-1478, PD153035 HCl, pelitinib, AC480, AEE788, AP261 13-analog, OSI-420, WZ3146, WZ8040, AST-1306, rociletinib, genisten, varlitinib, icotinib, TAK-285, WHI-P154, daphnetin, PD168393, tyrphostin9, CNX-2006, AG-18, AZ5104, osimertinib, CL-387785, olmutinib, AZD3759, poziotinib, vandetanib, and necitumumab.

In various aspects, the agent that modulates JAK3 signaling is a JAK3 inhibitor and wherein the agent that modulates EGFR signaling is an EGFR inhibitor.

In various aspects, the agent that modulates JAK3 signaling and the agent that modulates EGFR signaling are co-formulated. In various further aspects, the agent that modulates JAK3 signaling and the agent that modulates EGFR signaling are co-packaged.

In various aspects, the agent that modulates JAK3 signaling and the agent that modulates JAK3 signaling are administered concurrently. In various further aspects, the agent that modulates JAK3 signaling and the agent that modulates EGFR signaling are not administered concurrently.

In various aspects, the method further comprises administering to the subject an effective amount of an agent associated with the treatment of glioma. Examples of agents known for the treatment of glioma include, but are not limited to, temozolomide.

In various aspects, the agent that modulates JAK3 signaling is tofacitinib and wherein the agent associated with the treatment of glioma is temozolomide.

In various aspects, the agent that modulates JAK3 signaling and the agent associated with the treatment of glioma are co-formulated. In various further aspects, the agent that modulates JAK3 signaling and the agent associated with the treatment of glioma are co-packaged.

In various aspects, the agent that modulates JAK3 signaling and the agent associated with the treatment of glioma are administered concurrently. In various further aspects, the agent that modulates JAK3 signaling and the agent associated with the treatment of glioma are not administered concurrently.

In various aspects, the method further comprises administering to the subject an effective amount of an agent associated with the treatment of inflammation, such as, for example, a glucocorticoid. Examples of glucocorticoids include, but are not limited to, beclomethason, betamethasone, budesonide, cortisone, dexamethasone, hydrocortisone, methylprednisolone, prednisolone, prednisone, and triamcinolone.

In various aspects, the agent that modulates JAK3 signaling and the agent associated with the treatment of inflammation are co-formulated. In various further aspects, the agent that modulates JAK3 signaling and the agent associated with the treatment of inflammation are co-packaged.

In various aspects, the agent that modulates JAK3 signaling and the agent associated with the treatment of inflammation are administered concurrently. In various further aspects, the agent that modulates JAK3 signaling and the agent associated with the treatment of inflammation are not administered concurrently.

In various aspects, the method further comprises administering to the subject an effective amount of an EGFR ligand. Examples of EGFR ligands include, but are not limited to, EGF, TGFA, HB-EGF, AR, EREG, BTC, and EPGN. In a further aspect, the EGFR ligand is EGF.

In various aspects, the agent that modulates JAK3 signaling and the EGFR ligand are co-formulated. In various further aspects, the agent that modulates JAK3 signaling and the EGFR ligand are co-packaged.

In various aspects, the agent that modulates JAK3 signaling and the EGFR ligand are administered concurrently. In various further aspects, the agent that modulates JACKS signaling and the EGFR ligand are not administered concurrently.

In various aspects, the effective amount is a therapeutically effective amount. In a further aspect, the effective amount is a prophylactically effective amount.

In various aspects, the effective amount is a concentration of less than about 10 nM, less than about 9 nM, less than about 8 nM, less than about 7 nM, less than about 6 nM, less than about 5 nM, less than about 4 nM, less than about 3 nM, less than about 2 nM, less than about 1 nM, less than about 0.8 nM, less than about 0.4 nM, or less than about 0.2 nM. In a further aspect, the effective amount is a concentration of less than about 1 nM.

In a further aspect, the subject has been diagnosed with a need for treatment of glioma prior to the administering step. In a still further aspect, the subject is at risk for developing glioma prior to the administering step.

In a further aspect, the subject is a mammal. In a still further aspect, the mammal is a human.

In a further aspect, the method further comprises the step of identifying a subject in need of treatment of glioma.

In various aspects, the glioma is a glioblastoma.

In a further aspect, the glioma expresses EGFR wild type. In a still further aspect, the glioma expresses EGFR mutant. In yet a further aspect, the glioma is resistant to EGFR inhibition.

In a further aspect, the method further comprises the step of administering a therapeutically effective amount of at least one chemotherapeutic agent. In yet a further aspect, the chemotherapeutic agent is selected from an alkylating agent, an antimetabolite agent, an antineoplastic antibiotic agent, a mitotic inhibitor agent, and an mTor inhibitor agent.

In various aspects, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In various aspects, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In various aspects, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In various aspects, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In various aspects, the mTor inhibitor agent is selected from everolimus, siroliumus, and temsirolimus, or a pharmaceutically acceptable salt, hydrate, solvate, or polymorph thereof.

### D. METHODS FOR TREATING GLIOMA USING EGFR LIGANDS

In one aspect, disclosed are methods for treating a glioma in a subject in need thereof, the method comprising administering to the subject an agent that increases EGFR ligand.

In one aspect, disclosed are methods for treating an EGFR amplified glioma in a subject in need thereof, the method comprising administering to the subject tofacitinib and an EGFR ligand, wherein at least one of tofacitinib and the EGFR ligand is administered in an effective amount. In a further aspect, the effective amount is an individually effective amount of tofacitinib and/or the EGFR ligand. In a still further aspect, the effective amount is an individually effective amount of tofacitinib. In yet a further aspect, the effective amount is an individually effective amount of the EGFR ligand. In an even further aspect, the effective amount is a combinatorically effective amount of tofacitinib and the EGFR ligand.

In various aspects, the agent that increases EGFR ligand is an agent that modulates BIN3 signaling and/or JAK3 signaling. In various further aspects, the agent that increases EGFR ligand is tofacitinib.

In various aspects, the agent that increases EGFR ligand is an EGFR ligand. In a further aspect, the EGFR ligand is selected from EGF, TGFA, HB-EGF, AR, EREG, BTC, and EPGN. In a still further aspect, the EGFR ligand is EGF.

In various aspects, the method further comprises administering to the subject an effective amount of an agent that modulates epidermal growth factor receptor (EGFR) signaling, or a pharmaceutically acceptable salt thereof. In various further aspects, the agent that modulates EGFR signaling is an EGFR inhibitor. In a further aspect, the EGFR inhibitor is a tyrosine kinase inhibitor. Examples of tyrosine kinase inhibitors include, but are not limited to, erlotinib. In a still further aspect, the EGFR inhibitor is a monoclonal antibody.

In various aspects, the EGFR inhibitor is selected from erlotinib, afatinib, cetuximab, panitumumab, erlotinib HCl, gefitinib, lapatinib, neratinib, lifirafenib, HER2-inhibitor-1, nazartinib, naquotinib, canertinib, AG-490, CP-724714, Dacomitinib, WZ4002, Sapitinib, CUDC-101, AG-1478, PD153035 HCl, pelitinib, AC480, AEE788, AP261 13-analog, OSI-420, WZ3146, WZ8040, AST-1306, rociletinib, genisten, varlitinib, icotinib, TAK-285, WHI-P154, daphnetin, PD168393, tyrphostin9, CNX-2006, AG-18, AZ5104, osimertinib, CL-387785, olmutinib, AZD3759, poziotinib, vandetanib, and necitumumab.

In various aspects, the agent that increases EGFR ligand and the agent that modulates EGFR signaling are co-formulated. In various further aspects, the agent that increases EGFR ligand and the agent that modulates EGFR signaling are co-packaged.

In various aspects, the agent that increases EGFR ligand and the agent that modulates EGFR signaling are administered concurrently. In various further aspects, the agent that increases EGFR ligand and the agent that modulates EGFR signaling are not administered concurrently.

In various aspects, the method further comprises administering to the subject an effective amount of an agent associated with the treatment of glioma. Examples of agents known for the treatment of glioma include, but are not limited to, tumor treating fields, bevacizumab, and radiation therapy. In a further aspect, the agent associated with the treatment of glioma is temozolomide.

In various aspects, the agent that increases EGFR ligand and the agent associated with the treatment of glioma are co-formulated. In various further aspects, the agent that increases EGFR ligand and the agent associated with the treatment of glioma are co-packaged.

In various aspects, the agent that increases EGFR ligand and the agent associated with the treatment of glioma are administered concurrently. In various further aspects, the agent that increases EGFR ligand and the agent associated with the treatment of glioma are not administered concurrently.

In various aspects, the method further comprises administering to the subject an effective amount of an agent associated with the treatment of inflammation, such as, for example, a glucocorticoid. Examples of glucocorticoids include, but are not limited to, beclomethason, betamethasone, budesonide, cortisone, dexamethasone, hydrocortisone, methylprednisolone, prednisolone, prednisone, and triamcinolone.

In various aspects, the agent that increases EGFR ligand and the agent associated with the treatment of inflammation are co-formulated. In various further aspects, the agent that increases EGFR ligand and the agent associated with the treatment of inflammation are co-packaged.

In various aspects, the agent that increases EGFR ligand and the agent associated with the treatment of inflammation are administered concurrently. In various further aspects, the agent that increases EGFR ligand and the agent associated with the treatment of inflammation are not administered concurrently.

In various aspects, the method comprises administering tofacitinib and an EGFR ligand. In a further aspect, the EGFR ligand is selected from EGF, TGFA, HB-EGF, AR, EREG, BTC, and EPGN. In a further aspect, the EGFR ligand is EGF. In a still further aspect, each of tofacitinib and the EGFR ligand is administered in an effective amount.

In various aspects, the agent that modulates BIN3 signaling and the EGFR ligand are co-formulated. In a still further aspect, the agent that modulates BIN3 signaling and the EGFR ligand are co-packaged.

In various aspects, tofacitinib and the EGFR ligand are administered concurrently. In various further aspects, tofacitinib and the EGFR ligand are not administered concurrently.

In various aspects, the effective amount is a therapeutically effective amount. In a further aspect, the effective amount is a prophylactically effective amount.

In various aspects, the effective amount is a concentration of less than about 10 nM, less than about 9 nM, less than about 8 nM, less than about 7 nM, less than about 6 nM, less than about 5 nM, less than about 4 nM, less than about 3 nM, less than about 2 nM, less than about 1 nM, less than about 0.8 nM, less than about 0.4 nM, or less than about 0.2 nM. In a further aspect, the effective amount is a concentration of less than about 1 nM.

In a further aspect, the subject has been diagnosed with a need for treatment of glioma prior to the administering step. In a still further aspect, the subject is at risk for developing glioma prior to the administering step.

In a further aspect, the subject is a mammal. In a still further aspect, the mammal is a human.

In a further aspect, the method further comprises the step of identifying a subject in need of treatment of glioma.

In various aspects, the glioma is a glioblastoma.

In a further aspect, the glioma expresses EGFR wild type. In a still further aspect, the glioma expresses EGFR mutant. In yet a further aspect, the glioma is resistant to EGFR inhibition. In an even further aspect, the glioma is an EGFR amplified glioma. Thus, for example, the glioma can carry at least 5% more copies, at least 10% more copies, at least 15% more copies, at least 20% more copies, at least 25% more copies, at least 30% more copies, at least 35% more copies, at least 40% more copies, at least 45% more copies, at least 50% more copies, at least 55% more copies, at least 60% more copies, at least 65% more copies, at least 70% more copies, at least 75% more copies, at least 80% more copies, at least 85% more copies, at least 90% more copies, or at least 95% more copies relative to normal cells.

In a further aspect, the method further comprises the step of administering a therapeutically effective amount of at least one chemotherapeutic agent. In yet a further aspect, the chemotherapeutic agent is selected from an alkylating agent, an antimetabolite agent, an antineoplastic antibiotic agent, a mitotic inhibitor agent, and an mTor inhibitor agent.

In various aspects, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In various aspects, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In various aspects, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In various aspects, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In various aspects, the mTor inhibitor agent is selected from everolimus, siroliumus, and temsirolimus, or a pharmaceutically acceptable salt, hydrate, solvate, or polymorph thereof.

### E. ADDITIONAL METHODS OF USING THE COMPOUNDS

The compounds and pharmaceutical compositions of the invention are useful in treating or controlling gliomas such as, for example, malignant gliomas.

To treat or control glioma, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of glioma.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of glioma.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Use of Agents and Compositions

In one aspect, the invention relates to the use of a disclosed agent, a disclosed pharmaceutical composition, or a product of a disclosed method. In a further aspect, a use relates to the manufacture of a medicament for the treatment of glioma in a subject.

Also provided are the uses of the disclosed agents, compositions, and products. In one aspect, the invention relates to use of at least one disclosed agent, or a pharmaceutically acceptable salt, hydrate, solvate, or polymorph thereof, or at least one disclosed composition. In a further aspect, the composition used is a product of a disclosed method of making.

In a further aspect, the use relates to a process for preparing a pharmaceutical composition comprising a therapeutically effective amount of a disclosed agent or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed agent or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of glioma in a subject. In one aspect, the use is characterized in that the subject is a human. In one aspect, the use is characterized in that the glioma is a malignant glioma.

In a further aspect, the use relates to the manufacture of a medicament for the treatment of glioma in a subject.

It is understood that the disclosed uses can be employed in connection with the disclosed agents, products of disclosed methods of making, methods, compositions, and kits. In a further aspect, the invention relates to the use of a disclosed agents or a disclosed product in the manufacture of a medicament for the treatment of glioma in a mammal. In a further aspect, the glioma is a malignant glioma.

2. Manufacture of a Medicament

In one aspect, the invention relates to a method for the manufacture of a medicament for treating glioma in a subject in need thereof, the method comprising combining a therapeutically effective amount of a disclosed agent, composition, or product of a disclosed method with a pharmaceutically acceptable carrier or diluent.

As regards these applications, the present method includes the administration to an animal, particularly a mammal, and more particularly a human, of a therapeutically effective amount of the agents effective in the treatment of glioma. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal and the body weight of the animal.

The total amount of the agent of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the agent or composition and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed agent, composition, or a product of a disclosed method of making, or a pharmaceutically acceptable salt, solvate, or polymorph thereof, with a pharmaceutically acceptable carrier or diluent.

3. Kits

In one aspect, disclosed are kits comprising an agent that modulates BIN3 signaling, or a pharmaceutically acceptable salt thereof, and one or more of: (a) an agent associated with the treatment of cancer; (b) an agent associated with the treatment of inflammation; (c) instructions for administering the agent that modulates BIN3 signaling in connection with treating glioma; and (d) instructions for treating glioma.

In one aspect, disclosed are kits comprising an agent that modulates JAK3 signaling, or a pharmaceutically acceptable salt thereof, and one or more of: (a) an agent associated with the treatment of cancer; (b) an agent associated with the treatment of inflammation; (c) instructions for administering the agent that modulates JAK3 signaling in connection with treating glioma; and (d) instructions for treating glioma.

In various aspects, the agent that modulates BIN3 signaling is tofacitinib. In various aspects, the agent that modulates JAK3 signaling is tofacitinib.

In a further aspect, the glioma is a malignant glioma.

In a further aspect, the agent associated with the treatment of cancer is an EGFR inhibitor.

In a further aspect, the agent associated with the treatment of cancer is a chemotherapeutic agent. In yet a further aspect, the chemotherapeutic agent is selected from an alkylating agent, an antimetabolite agent, an antineoplastic antibiotic agent, a mitotic inhibitor agent, and an mTor inhibitor agent.

In various aspects, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In various aspects, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In various aspects, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In various aspects, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In various aspects, the mTor inhibitor agent is selected from everolimus, siroliumus, and temsirolimus, or a pharmaceutically acceptable salt, hydrate, solvate, or polymorph thereof.

In a further aspect, the agent associated with the treatment of cancer is associated with the treatment of glioma. In a still further aspect, the agent associated with the treatment of glioma is temozolomide.

In a further aspect, the agent that modulates BIN3 signaling and the agent associated with the treatment of cancer are co-packaged. In a still further aspect, the agent that modulates BIN3 signaling and the agent associated with the treatment of cancer are co-formulated.

In a further aspect, the agent that modulates JAK3 signaling and the agent associated with the treatment of cancer are co-packaged. In a still further aspect, the agent that modulates JAK3 signaling and the agent associated with the treatment of cancer are co-formulated.

In a further aspect, the agent that modulates BIN3 signaling and the agent associated with the treatment of cancer are administered sequentially. In a still further aspect, the agent that modulates BIN3 signaling and the agent associated with the treatment of cancer are administered simultaneously.

In a further aspect, the agent that modulates JAK3 signaling and the agent associated with the treatment of cancer are administered sequentially. In a still further aspect, the agent that modulates JAK3 signaling and the agent associated with the treatment of cancer are administered simultaneously.

In various aspects, the agent associated with the treatment of inflammation is a glucocorticoid. Examples of glucocorticoids include, but are not limited to, beclomethason, betamethasone, budesonide, cortisone, dexamethasone, hydrocortisone, methylprednisolone, prednisolone, prednisone, and triamcinolone.

In a further aspect, the agent that modulates BIN3 signaling and the agent associated with the treatment of inflammation are co-packaged. In a still further aspect, the agent that modulates BIN3 signaling and the agent associated with the treatment of inflammation are co-formulated.

In a further aspect, the agent that modulates JAK3 signaling and the agent associated with the treatment of inflammation are co-packaged. In a still further aspect, the agent that modulates JAK3 signaling and the agent associated with the treatment of inflammation are co-formulated.

In a further aspect, the agent that modulates BIN3 signaling and the agent associated with the treatment of inflammation are administered sequentially. In a still further aspect, the agent that modulates BIN3 signaling and the agent associated with the treatment of inflammation are administered simultaneously.

In a further aspect, the agent that modulates JAK3 signaling and the agent associated with the treatment of inflammation are administered sequentially. In a still further aspect, the agent that modulates JAK3 signaling and the agent associated with the treatment of inflammation are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Methods

a. Cell Culture of Mayo PDXS

Mayo PDX cells were cultured in DMEM with 10% FBS, 1% P/S. Cells were grown to 70-80% confluency prior to treatments. All PDXs were authenticated using short-tandem repeat profiling by the Mayo Clinic Brain Tumor Patient-Derived Xenograft National Resource.

b. Plasmids, Transfection, and Generation of Stable Cell Lines

To generate cells stably expressing TGFα or BIN3, PDXs were cultured in six-well plate and transfected with either 2 μg of pCMV-TGFα-Flag (Sino Biological, China) or pCMV-BIN3-HA (Sino Biological, China) or empty vector using Lipofectamine 2000 according to manufacturer's instructions. 48 hours after transfection, the cells were selected for transfection positivity by hygromycin (200 μg/ml) selection. Stable expression colonies were selected and tested for BIN3 or TGFα expression by immunoblotting. Positive clones were selected for further investigation. Full-length EGR1 promoter-reporter plasmid was a gift.

C. Western Blotting, Antibodies, and Reagents

Whole protein extracts from cells or tumor tissues were analyzed by western blot as previously described (Altieri et al. (2015) Surg. Technol. Int 27: 297-302). Mayo PDX cells were serum starved overnight. Following treatment for specific time, cells were harvested and lysed. The human glioblastoma tissues were ground in liquid nitrogen and then were lysed. EGFR (06-847) antibody was from Millipore. DOCK7 was from Proteintech (Rosemont, Ill.). pEGFR (2236), STAT3 (12640), pSTAT3 (9145), cdc42(4376), and RhoA (4695) antibodies were from Cell Signaling Technology (Danvers, Mass.). TGFα antibody was from R&D system. BIN3 (sc-514396) and (3-Actin (sc-47778) were from Santa Cruz Biotechnology (Dallas, Tex.).

Reagents: Recombinant human EGF (AF-100) was obtained from Peprotech (Rocky Hill, N.J.). Recombinant human HB-EGF (259-HE) and TGFα (230-A) were obtained from R&D systems. Erlotinib (S7786) was purchased from SelleckChem (Houston, Tex.). Jak inhibitor tofacitinib and ERK inhibitor (U0126) were from Cayman Chemical (Ann Arbor, Mich.). The JNK inhibitor SP600125, Met inhibitor SU11274 and NF-□B inhibitor BMS-345541 were obtained from EMD Millipore (Billerica, Mass.).

d. Matrigel Invasion Assay

Invasion status of cells was tested with Matrigel Boyden chamber assays (Fisher). 1×105 cells were plated on BD BioCoat Matrigel invasion chambers. 24 hours after treatment with drugs, invaded cells were stained with the HEMA-3 kit (Fisher). Invaded cells were counted in 4-5 random fields.

e. Scratch-Wound Assays

Cells were cultured in 6 well plate until they reached around 100% confluence. A scratch wound was produced on the monolayer using a sterile 200 ul pipette tip. The cell cultured with serum free DMEM containing EGF (50 ng/ml) or vehicle control. Images of wound were captured 0 and 24 hours after scratch generation.

f. Bromodeoxyuridine Cell Proliferation Assay

Cell proliferation was assessed using the bromodeoxyuridine (BrdU) cell proliferation enzyme-linked immunosorbent assay kit (Abcam, Cambridge, United Kingdom) according to the manufacturer's instructions. Cells were plated in 96-well plates (1×104 cells/well) and serum starved overnight, followed by treatment with or without 50 ng/ml EGF or tofacitinib (1 μM) for 48 hours before the assay.

g. Immunoprecipitation, Mass Spectrometry, and Chromatin Immunoprecipitation

Cells were treated with 50 ng/ml EGF for 24 hours, BIN3 (sc-514396AC, Santa Cruz Biotechnology) or mouse IgG antibodies were incubated with whole cell lysates overnight at 4° C., and the mixtures were then incubated with 40 μl protein A/G slurry beads (Sigma) for 2 hours at 4° C. Beads were washed three times with lysis buffer, and immunoprecipitates were boiled in loading buffer for 5 min, subjected to SDS-PAGE followed by Coomassie stain. Bands were cut from the gel and submitted for Mass Spectrometry Facility, UTSW, Dallas. To identify BIN3 and DOCK7 complexes in PDXs upon EGF treatment, cell lysates were immunoprecipitated with DOCK7 antibody or BIN3 antibody, and analyzed by Western blot using BIN3 antibody.

Chromatin immunoprecipitation (ChIP) assays were performed using a ChIP assay kit (Upstate Biotechnology, Lake Placid, N.Y., USA). Cells were treated with or without EGF for the indicated time points or transfected with indicated vectors for 48 hours followed by cell fixation, lysis, chromatin shearing, antibody incubation and washing according to the manufacturer's protocol. ChIP grade anti-EGR1 (4153) or anti-Nanog antibody (5232) antibody from Cell Signaling Technology (Danvers, Mass.) was used to selectively precipitate the corresponding protein-DNA complex. RT-PCR was performed using ViiA 7 Real-Time PCR System (Applied Biosystems) to measure the relative amounts of ChIP DNA and results were quantified relative to inputs. The data are expressed as percentage of input. Following are primer sets: BIN3_Forward: 5′-TTGCAGCCTGTGTGTCTAAG-3′ (SEQ ID NO:1); BIN3_Reverse: 5′-CTCCAGGAAGTGACGTAAGC-3′ (SEQ ID NO:2); EMP1_Forward: 5′-AAAGTGGATACAGAGACA-3′ (SEQ ID NO:3); EMP1_Reverse: 5′-GTGAAAAACATCTGGCCA-3′ (SEQ ID NO:4).

h. Analysis of RhoA, Cdc42, and DOCK7 Activity

The amount of GTP-bound RhoA and Cdc42 were measured using RhoA and Cdc42 pull down assay kit (Cell Signaling) according to the protocol provided by the manufacturer. Briefly, cell lysates were incubated with either glutathione S-transferase (GST)-Rhotekin-RBD or GST-PAK-PBD beads 4° C. for 1 hour to pull down GTP-bound RhoA or Cdc42. Beads were washed four times in washing buffer and re-suspended in lysis buffer. RhoA-GTP and Cdc42-GTP were detected by Western blot using RhoA and Cdc42 antibodies.

To measure GEF activity of DOCK7, cell lysates were incubated with 40 μl Cdc42 G15A agarose beads (ab211185, Abcam) at 4° C. for 1 hour. The agarose beads were then washed boiled and the supernatants were used to immunoblot with DOCK7 antibody to determine levels of active DOCK7.

i. ELISA

To determine EGFR ligands in medium, cells were serum starved for 48 hours, supernatants were collected and concentrated 5- to 10-fold with Pierce protein concentrator (ThermoFisher). HB-EGF, TGFα and EGF protein concentration in supernatant and tumor tissue extracts was determined by ELISA using the corresponding commercial HB-EGF, TGFα and EGF protein detection kits (ThermoFisher) per the manufacturer's instructions.

j. Small Interfering RNA (siRNA) and Lentiviral-Mediated Short Hairpin RNA (shRNA) Knockdown

Human BIN3 (sc-77692), HB-EGF (sc-39420), EGFR (sc-29301), DOCK7(sc-105312), RhoA (sc-29471), CDC42 (sc-29256) and scrambled siRNAs were obtained from Santa Cruz Biotechnology (Dallas, Tex.). PDX cells were seeded in six well plates and transfected with the siRNA pool using Lipofectamine2000 (Invitrogen Carlsbad, Calif.). Experiments were conducted 48 hours after siRNA transfection. Knockdown efficiency was confirmed by Western blot.

Human HB-EGF shRNA lentiviral particles (sc-39420-V) and control lentiviral particles were purchased from Santa Cruz Biotechnology (Dallas, Tex.). GBM39 cells were plated in a six well plate and infected with HB-EGF shRNA or control lentiviral particles in the presence of polybrene for 24 hour. The cells were then incubated with fresh medium for additional 24 hours. The transfected GBM39 cells were sub cultured in fresh DMEM containing 1 mg/ml puromycin. Clones were isolated and expanded, knockdown efficiency was determined by HB-EGF ELISA kit.

k. cDNA Synthesis and Real Time PCR

Total RNA from cells was extracted by TRIzol Reagent (Ambion). First-strand cDNA and PCR was performed as described previously (Altieri et al. (2015) Surg. Technol. Int. 27: 297-302). The expression of each gene was normalized to GAPDH as a reference. The following primers were used. BIN3, 5′-CCCAGGGACCTCTCTCTAATCA-3′ (SEQ ID NO:5) and 5′-GCTACAGGCTTGTCACTCGG-3′ (SEQ ID NO:6); GAPDH, 5′-GTGAAGGTCGGAGTCAACGG-3′ (SEQ ID NO:7) and 5′-TGATG-ACAAGCTTCCCGTTCTC-3′ (SEQ ID NO:8).

1. Single Cell Migration Assay

For time-lapse analysis of individual cell movement, nano-ridge constructed of transparent poly(urethane acrylate) (PUA), and fabricated using UV-assisted capillary lithography as described by Kim et al. were used (Kim, D et al. (2009) Mechanosensitivity of fibroblast cell shape and movement to anisotropic substratum topography gradients, Biomaterials 30: 5433-5444; Kim, et al. (2009) Guided Cell Migration on Microtextured Substrates with Variable Local Density and Anisotropy, Adv Funct Mater 19: 1579-1586; Garzon-Muvdi, et al. (2012) Regulation of brain tumor dispersal by NKCC1 through a novel role in focal adhesion regulation, PLoS Biol 10: e1001320). The 24 well NanoSurface plate was purchased from Curi Bio (Seattle, Wash.). Prior to cell seeding, nanoridges surfaces were coated with laminin (3 μg/cm², Sigma-Aldrich). Cells were plated at low density (1×104 cells/ml) and incubated at 37° C. overnight. The following day, cells were washed with PBS and cultured in DMEM supplemented with 1% FBS and were ready for time-lapse imaging. To measure cell proliferation, 6 hours after plating Histone2B-GFP (C10594, ThermoFisher) was added for nuclear staining overnight. The next day cells were incubated with a mixture of BioTracker® NTP-Transporter Molecule (SCT064, Sigma Aldrich) and Cy3-dUTP (50-190-5459, ThermoFisher) for 10 minutes and then replaced with 1% FBS media containing HB2B2-GFP. For time-lapse imaging, the plate was mounted onto the stage of Andor spinning disk confocal microscope equipped with temperature and CO₂ controlling environmental chamber. Six hours after the start of the imaging, EGF (50 ng/ml) or tofacitinib (1 μM) was added to each well without moving the plate. Both fluorescent and bright-field images were taken every 30 minutes for 24 hours using a 10× objective. ImageJ manual track Plugin was used to track the movement of cells frame by frame.

m. Immunohistochemistry and Immunofluorescence

For immunohistochemistry, tumor tissues were fixed in 10% formalin and embedded in paraffin. Immunohistochemistry analysis was performed using the ABC streptavidin-biotin method with the Vectastain ABC kit (Vector Laboratories, Burlingame, Calif., USA) as described previously (Altieri et al. (2015) Surg. Technol. Int 27: 297-302). Following primary antibodies are used: HB-EGF (1:500, sc-365812, Santa Cruz Biotechnology), TGFα (1:200, R&D systems) and SMI-31 (1:500, R&D systems). All antibodies besides SMI-31 were incubated overnight at four degree. SMI-31 was incubated at room temperature for 2 hours. Three to four complete and non-overlapping high magnification (×400) fields were randomly selected for each section. For Ki-67, the percentage of positively stained nuclei out of the total cells counted was evaluated.

Human glioblastoma slides stained with antibodies to HB-EGF and TGFα were evaluated under microscope for signal intensity. The immunostaining score ranges from 0 to 3 based on percentage positive staining (0: 5%, 1: 5%-30%, 2: 30%-70%, 3: >70%). 3 high cellular (tumor central) or low cellular (invasive) regions per each tumor sections were selected for evaluation. The Wilcoxon rank-sum test was applied to test the significant differences in immunohistochemical staining intensity between two regions.

For immunofluorescence, cells were plated and cultured in the 24 well NanoSurface plate as described in the Single Cell migration assay. Cells were treated with EGF (50 ng/ml) for 24 hours and were fixed with 4% PFA followed by cell membrane permeabilization in 0.5% Triton X-100/PBS and blocked in 1% BSA/PBS. The primary antibody for detecting Ki67 was from Cell Signaling (9129). Alexa555-conjugated secondary antibody (Cell Signaling, 4413) was used. Cell nuclei were counterstained with DAPI (0.1 μg/ml).

n. Luciferase Assays

Cells were plated in 48 well dishes to 70%-80% confluence followed by transfection with EGR1 promoter plasmid or empty plasmid using lipofectamine 2000. Renilla luciferase was co-transfected as an internal control. A dual-luciferase reporter assay system was used according to manufacturer's instructions (Promega, Madison Wis.). Firefly luciferase activity was measured in a luminometer and normalized on the basis of Renilla luciferase activity.

o. Animal Studies

4 to 6 weeks old female athymic nude mice were purchased from Charles River Laboratories. Mayo PDXs were injected into the right corpus striatum of the brains of 6-8 week-old nude mice using a stereotactic frame. For survival experiments, mice were randomly divided into two groups (6-8 mice per group) treated with vehicle or tofacitinib by oral gavage throughout the entire experiment. Kaplan-Maier survival curves were calculated using GraphPad Prism 7.0 software. Alzet Osmotic pumps were installed for brain delivery of EGF or vehicle in tumor bearing mice. To monitor the mice with orthotropic xenografts, MRI was performed at the Mouse MRI Core, Advanced Imaging Research Center, at UT Southwestern.

Two-photon laser intravital microscope combined with cranial window surgery was used to observe cellular movement in vivo. Female athymic nude mice were anesthetized with 2.5% isoflourane and secured in a stereotactic frame. The head was shaved and the scalp was cut in a circular manner. The periosteum was scraped and a drill was used to create a circular cranial window over the front and parietal bone. 10 μL of 1×10⁶ cells/ml GBM12 stably expressing H2B-GFP were implanted at a depth of 0.5 mm. Following implantation, a 5 mm silicone-based polydimethylsiloxane (PDMS) coverslip was glued to the bone surrounding the cranial window. PDMS (SYLGARD184, Sigma-Aldrich) film was prepared as described by Heo et al. (Heo, et al. (2016) A soft, transparent, freely accessible cranial window for chronic imaging and electrophysiology, Sci Rep 6: 27818). PDMS coverslip allows for easy insertion of Hamilton needle into the brain tissue for EGF delivery. Dental cement was applied on the skull surface, covering the edge of the coverslip. Seven days after surgery and 24 hours prior to imaging procedure, 10 ng EGF or vehicle was injected into the mice brain through cranial window. For in vivo imaging, mice were anesthetized with ketamine-xlazine (100 mg/kg, 10 mg/kg) and mouse head was stabilized using a customized head frame. Time-lapse z-stack images of the tumor were acquired at with a time interval of 10 minutes for 2 hours using upright Zeiss LSM780 confocal/multiphoto microscope. Images were imported into Imaris 9.0 for creation of 3D representations and cell movements were tracked using Imaris spot detection function.

All animal studies were done under Institutional Animal Care and Use Committee-approved protocols.

p. Data Analysis of Public Database

The The TCGA-GBM clinical data was downloaded from The Cancer Genome Atlas (TCGA, https://portal.gdc.cancer.gov/). The GBM RNA-sequencing (RNA-seq) data was downloaded from UCSC Xena browser (https://xena.ucsc.edu). EGFR and pEGFR protein expression data were acquired from the Cancer Proteome Atlas (TCPA, https://tcpaportal.org.). TCGA RNA-seq datasets and corresponding survival data for patients with colon, liver, or stomach cancer were downloaded from UCSC Xena browser. Log 2 (UQ-FPKM+1) conversion was performed for all RNA-seq data. The GBM subtype information was acquired from GlioVis (https://gliovis.bioinfo.cnio.es). Kaplan-Meier survival curves were constructed and compared by Gehan's or log rank test. The correlation coefficient between BIN3 and HB-EGF mRNA levels were analyzed by Pearson's method.

q. Statistical Analysis

All data were analyzed for significance using GraphPad Prism 8.0 software. Data are presented as means±SEM of three independent experiments. Two-tailed unpaired Student's t-test were used for comparison of two data sets. For the analysis of cell velocity and Cy3 staining intensity, statistical comparison were made by Mann-Whitney test or two-way ANOVA test as indicated in the legend. Kaplan-Meier survival curves were constructed and compared by log-rank test and Gehan's test. The correlation between mRNA expression levels and protein expression level was analysed by Spearman correlation coefficient. At least 3 independent experiments were performed unless otherwise indicated. Fisher's exact test was used to determine association between immunostaining intensity of high and low cellular areas in human tumor sections. P<0.05 was considered statistically significant. * means that P<0.05, ** means that P<0.01, *** means that <0.001 and **** indicates any P value less than 0.0001.

2. Constitutive Vs. Ligand-Induced EGFR Signaling Elicits Distinct Biological Responses in Glioma Cells

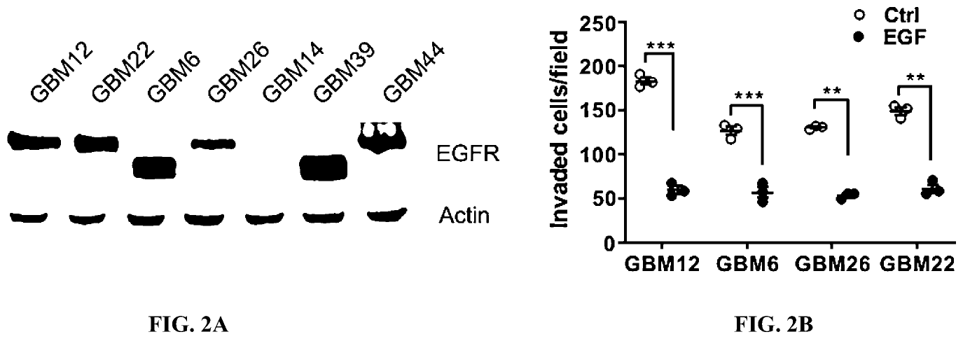

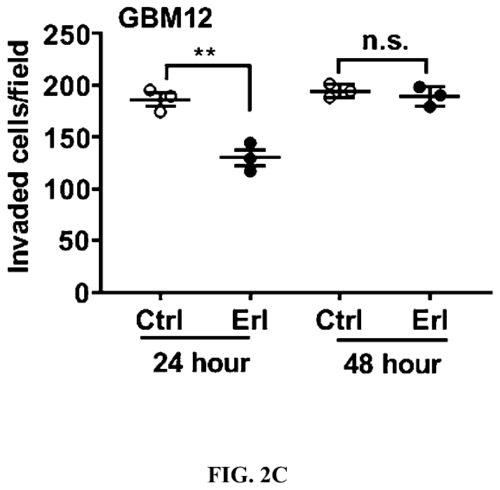

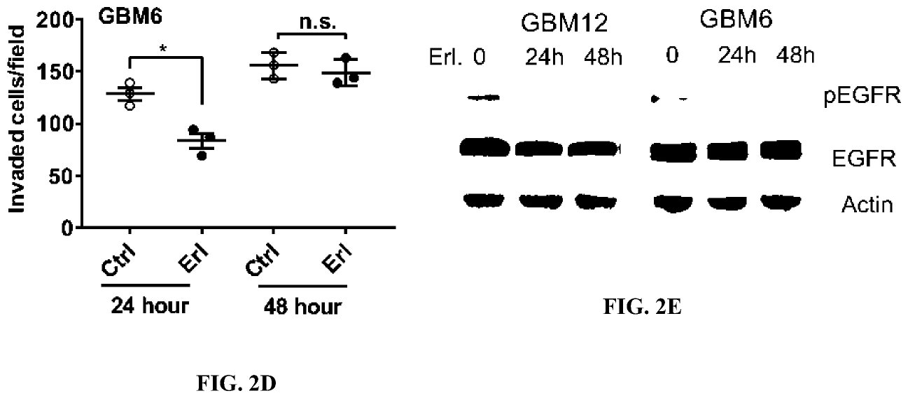

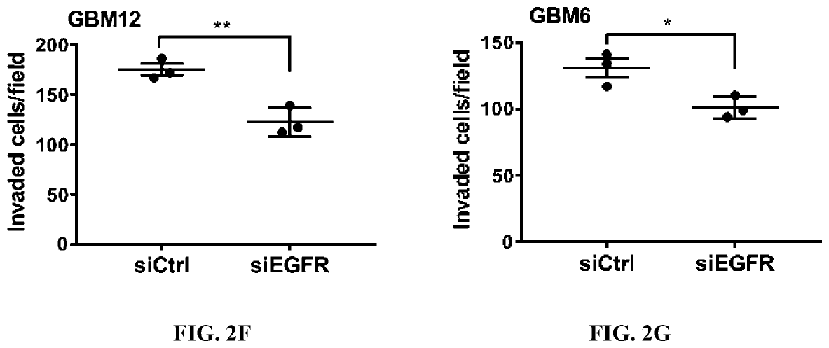

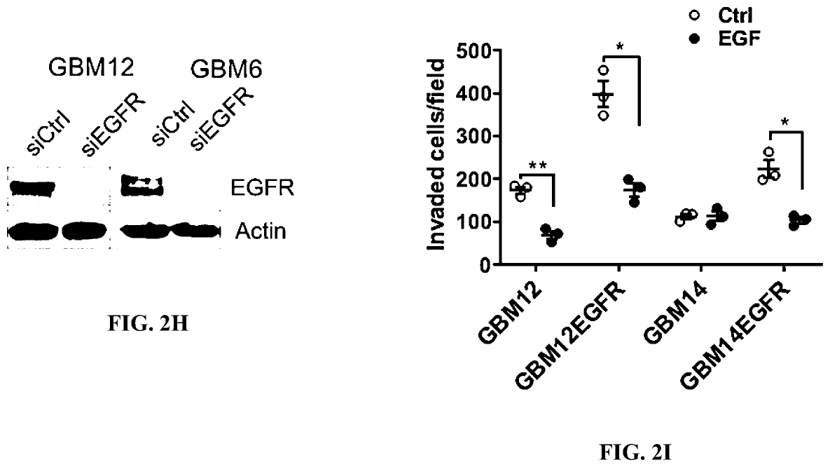

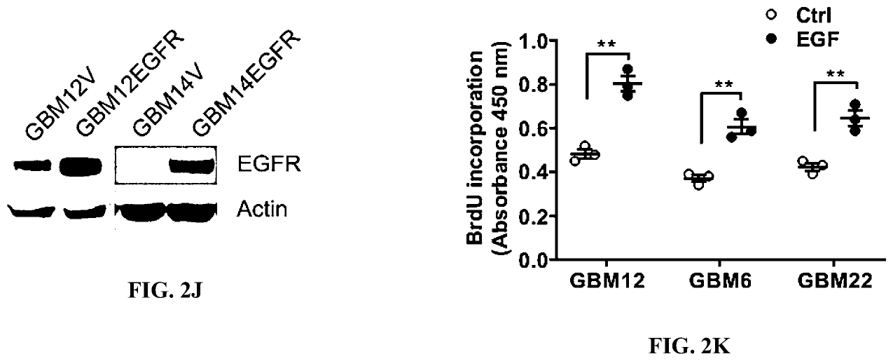

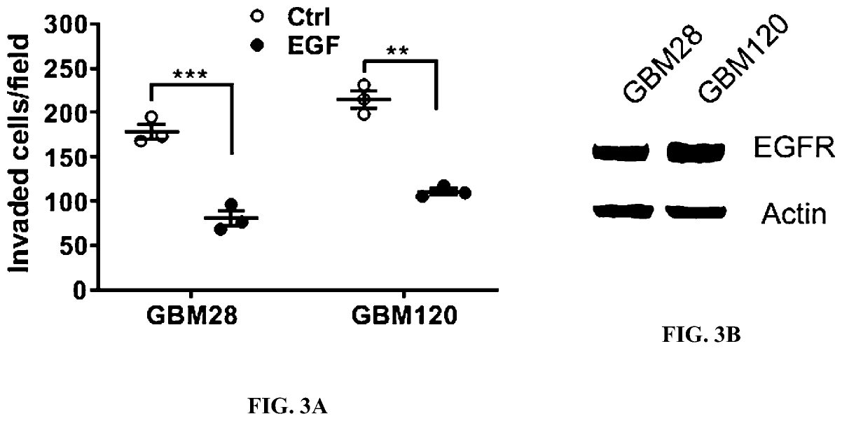

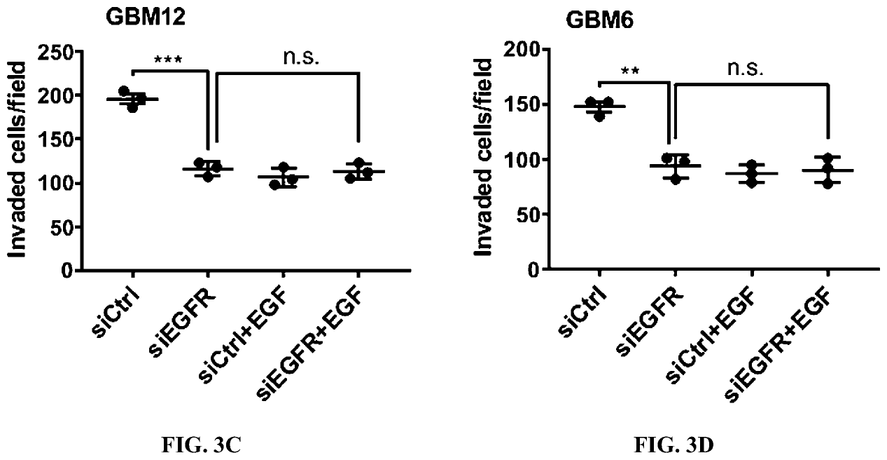

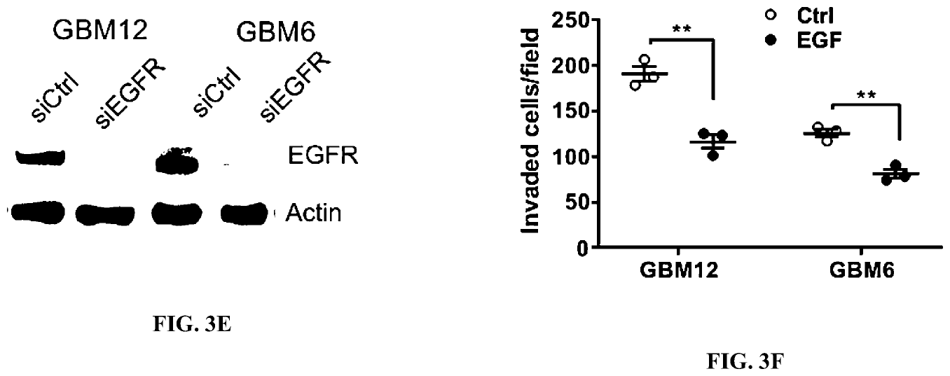

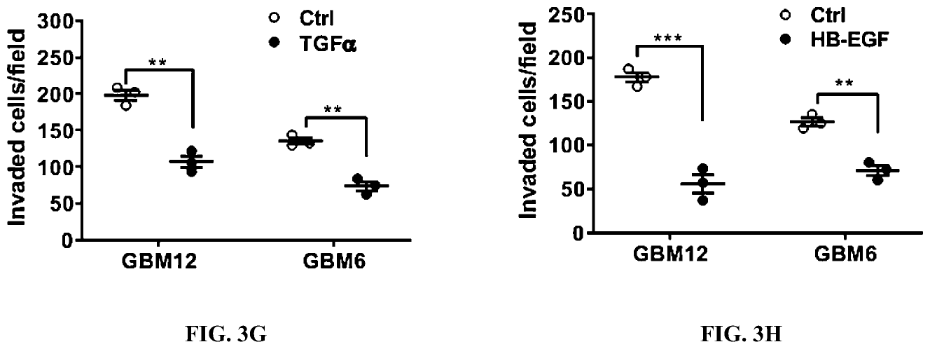

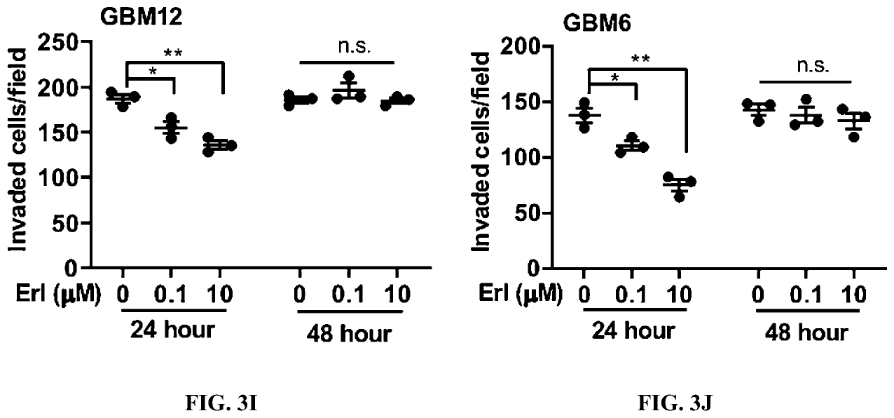

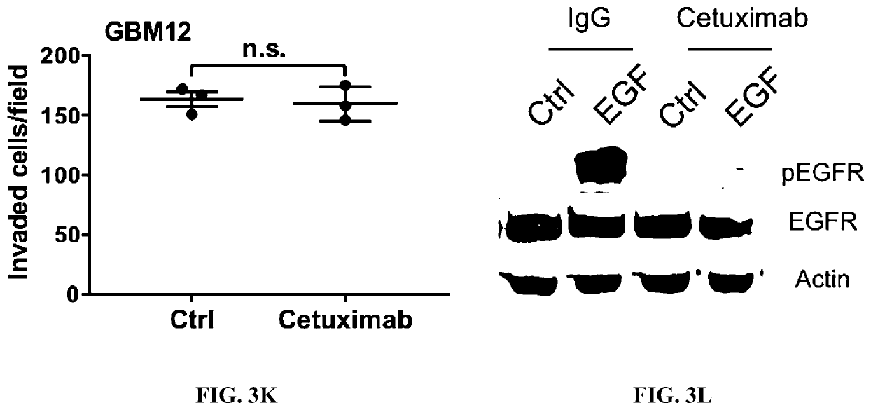

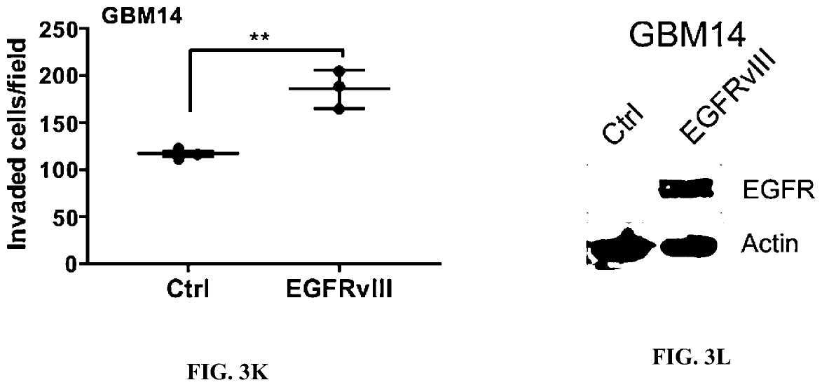

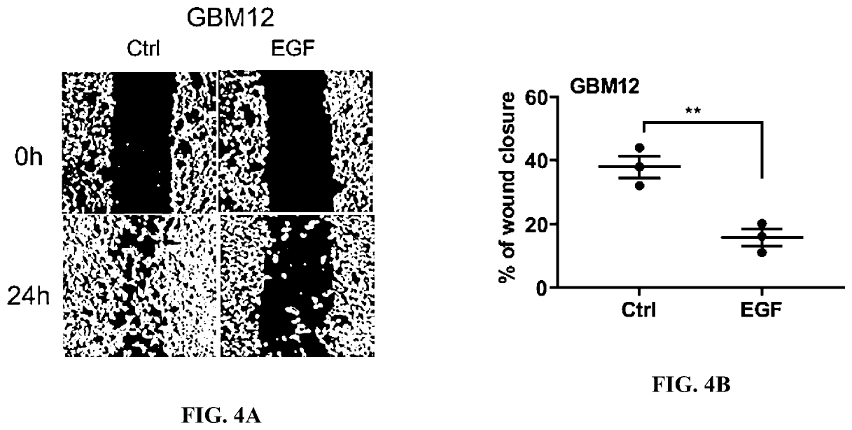

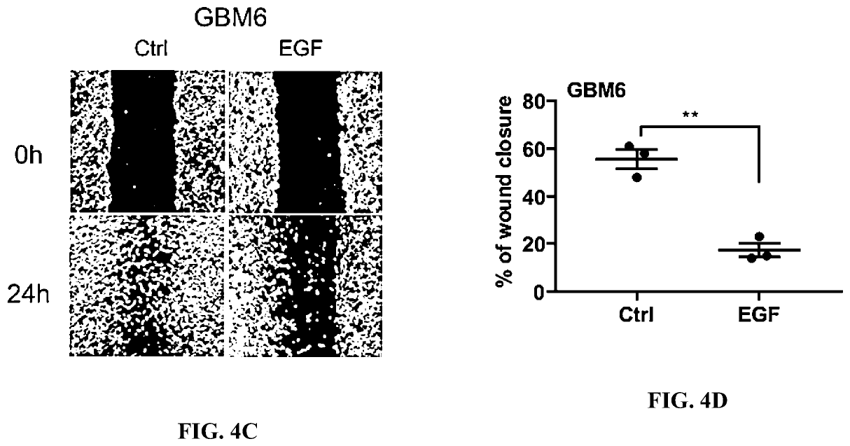

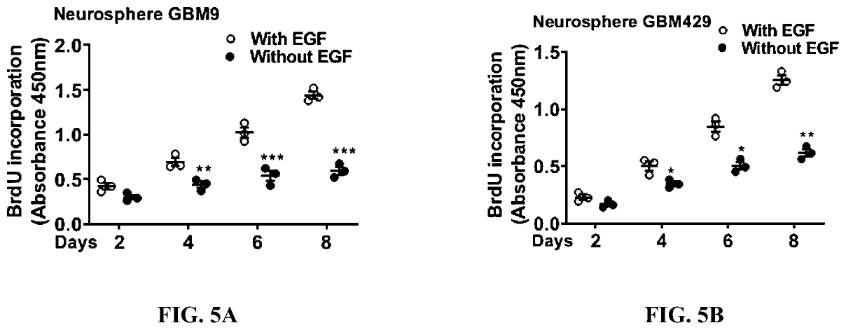

It was previously reported that constitutive vs. ligand-induced EGFRwt signaling trigger distinct downstream signaling cascades (Chakraborty et al. (2014) Nat. Commun. 5: 5811). To understand the biological consequences of constitutive vs. ligand induced EGFR mediated RTK signaling, the effect of EGF on glioma cell invasion was examined using a panel of well characterized Mayo patient derived xenograft (PDX) lines (Carlson et al. (2011) Curr. Protoc. Pharmacol. Chapter 14, Unit 14 16). The Mayo PDX lines tested express EGFRwt alone or EGFRvIII co-expressed with EGFRwt (FIG. 2A and Table 1). Surprisingly, it was found that addition of EGF suppressed invasion in multiple Mayo PDX explant cultures expressing EGFRwt or EGFRvIII plus EGFRwt in a transwell invasion assay (FIG. 2B, FIG. 3A, and FIG. 3B). siRNA knockdown of EGFR abolished the effectif of EGF on invasion indicating that the effect is mediated specifically through the EGFR (FIG. 3C-E). A low dose of EGF also suppressed invasion (FIG. 3F). Similar results were obtained with additional EGFR ligands, TGFα and HB-EGF (FIG. 3G-H). To determine whether EGFR activity is driving the basal invasiveness of these cells, the EGFR tyrosine kinase inhibitor erlotinib was used, and it was found that erlotinib suppressed the invasiveness of glioma cells suggesting that EGFR is driving the invasive phenotype (FIG. 2C and FIG. 2D). The effect of erlotinib is transient and invasion returns to baseline in 48 h despite continued suppression of EGFR activity (FIG. 2C, FIG. 2D, FIG. 3I, and FIG. 3J). The escape from erlotinib induced suppression is likely because of adaptive responses triggered in the cell resulting from EGFR inhibition (Guo et al. (2017) Nat. Neurosci. 20: 1074-1084; Sun and Bernards (2014) Trends in biochemical sciences 39: 465-474), since EGFR activation continues to be suppressed (FIG. 2E). Similarly, siRNA knockdown of EGFR inhibits invasiveness suggesting that ligand-independent EGFR activity is driving invasion in both EGFRwt and EGFRvIII expressing cells (FIG. 2F-H). To further confirm that constitutive EGFR activity drives invasion EGFRwt was overexpressed in PDX lines, including GBM14 that does not express endogenous EGFR and found that invasiveness is increased (FIG. 2I and FIG. 2J). If EGFR overexpressing cells are exposed to EGF, invasion is suppressed. Without wishing to be bound by theory, these data indicate that constitutive EGFR activity drives invasion while ligand-activated EGFR activity suppresses invasion. To rigorously rule out a role for autocrine/paracrine stimulation by EGF ligands in constitutive EGFRwt signaling, cetuximab was used, which blocks EGFR ligand binding, and showed that the invasiveness of GBM cells is unaffected by the addition of cetuximab while cetuximab completely blocks EGF-induced tyrosine phosphorylation of the EGFR (FIG. 3K and FIG. 3L). Additionally, it was shown that expression of the non-ligand binding EGFRvIII mutant in GBM14 lacking any EGFR expression also enhances invasion, ruling out intracellular autocrine activation and demonstrating ligand-independent constitutive EGFR activation enhances invasion (FIG. 3M and FIG. 3N). Scratch wound healing assay was also used as a second method of testing migration and results were found consistent with the transweel assays with ligand induced activation of the EGFR suppressing migration and constitutive EGFR signaling inducing migration (FIG. 4A-D). It should be noted that brain tumor initiating cells/neurosphere lines were not used for these experiments, because although neurospheres are primary cultures derived from GBMs, they are maintained in neural stem cell medium including a high concentration of EGF, rendering them unsuitable for experiments that require analysis of constitutive EGFR signaling. Attempts to culture several neurosphere lines with bFGF alone in the stem cell medium in the absence of EGF failed (FIG. 5A and FIG. 5B).

TABLE 1

| Mayo PDX | EGFR status |
|---|---|
| GBM6 | wt + vIII |
| GBM12 | wt |
| GBM22 | wt |
| GBM26 | wt |
| GBM39 | wt + vIII |
| GBM44 | wt |
| GBM14 | none |
| GBM28 | wt |
| GBM120 | wt |

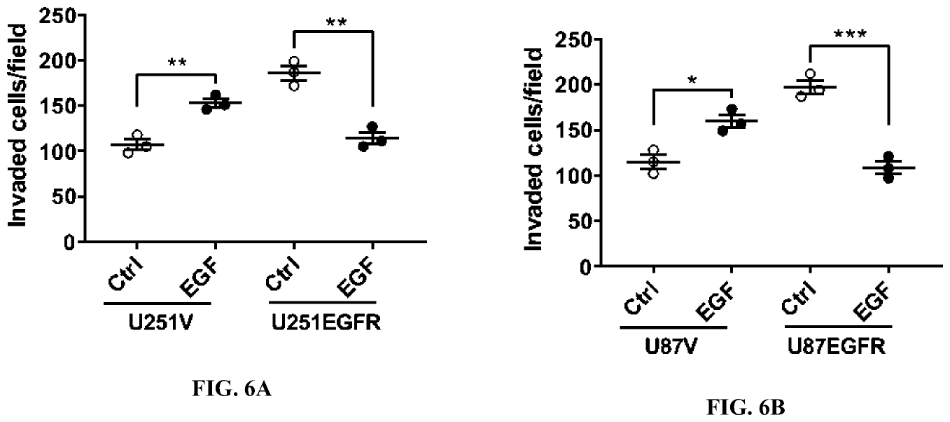

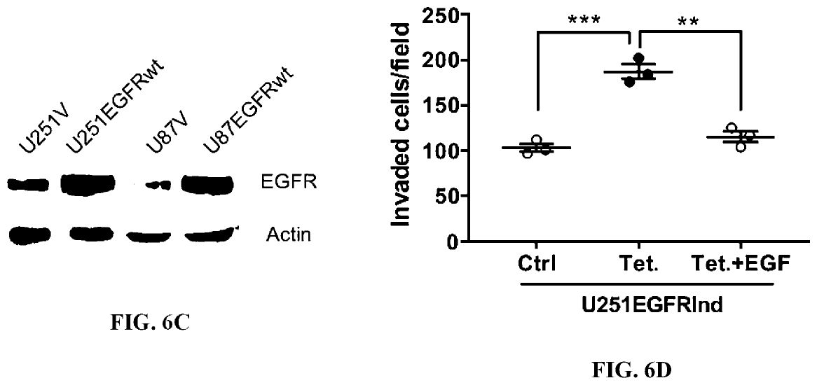

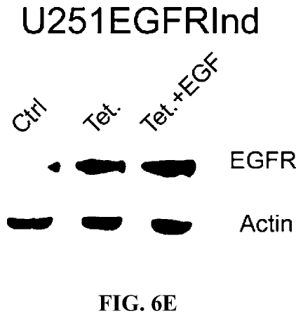

There are prior reports that suggest that addition of EGF results in increased invasiveness of glioma cell lines (Misek, et al. (2017) EGFR Signals through a DOCK180-MLK3 Axis to Drive Glioblastoma Cell Invasion, Mol Cancer Res 15: 1085-1095; Jiang, et al. (2014) PKM2 phosphorylates MLC2 and regulates cytokinesis of tumour cells, Nat Commun 5: 5566; Lund-Johansen, et al. (1990) Effect of epidermal growth factor on glioma cell growth, migration, and invasion in vitro, Cancer Res 50: 6039-6044; Westermark, et al. (1982) Effect of epidermal growth factor on membrane motility and cell locomotion in cultures of human clonal glioma cells, J Neurosci Res 8: 491-507). All of these studies have used established glioblastoma cell lines that are no longer considered to a representative model of GBM (Lee, et al. (2006) Tumor stem cells derived from glioblastomas cultured in bFGF and EGF more closely mirror the phenotype and genotype of primary tumors than do serum-cultured cell lines, Cancer Cell 9: 391-403). Additionally, it is known that established GBM cell lines lose the EGFR amplification. Thus, established GBM lines are considered a poor model for EGFR studies and the data derived from them are of uncertain value. Nonetheless, to address the effect of EGFR ligand reported in previous studies established GBM lines were tested. Consistent with prior studies, it was found that in U251MG and U87MG cell lines that express low levels of endogenous EGFR, exogenous EGF resulted in increased invasiveness (FIG. 6A-C). However, when the EGFR is overexpressed in U87MG and U251MG lines to levels comparable to GBM (Chakraborty, et al. (2014) Constitutive and ligand-induced EGFR signalling triggers distinct and mutually exclusive downstream signalling networks, Nat Commun 5: 5811; Puliyappadamba, et al. (2013) Opposing Effect of EGFRWT on EGFRvIII-Mediated NF-kappaB Activation with RIP1 as a Cell Death Switch, Cell Rep 4: 764-775), ligand addition resulted in decreased invasion (FIG. 6A-C). Similarly, inducible expression of EGFRwt in U251MG cells (Ramnarain, et al. (2006) Differential gene expression analysis reveals generation of an autocrine loop by a mutant epidermal growth factor receptor in glioma cells, Cancer Res 66: 867-874) resulted in increased invasion that is abolished upon addition of EGF (FIG. 6D and FIG. 6E). Most importantly, in multiple Mayo PDXs tested, which are more representative of human GBM60, EGF reduced invasiveness (FIG. 2B, FIG. 3A, and FIG. 3B). As demonstrated below, the inhibitory effect of EGFR ligand on invasion was also detected in multiple in vivo mouse models using several techniques. Thus, without wishing to be bound by theory, these data clearly demonstrate that EGF suppresses invasion in glioma cells. The effect of a growth factor to reduce invasion is not entirely unprecedented and has also been reported for VEGF particularly in the context of studies using the VEGF inhibitior bevacizumab (de Groot, et al. (2010) Tumor invasion after treatment of glioblastoma with bevacizumab: radiographic and pathologic correlation in humans and mice, Neuro Oncol 12: 233-242; Lu, et al. (2012) VEGF Inhibits Tumor Cell Invasion and Mesenchymal Transition through a MET/VEGFR2 Complex, Cancer Cell 22: 21-35).

Unlike its effect on invasion, it was found that ligand-induced EGFR activation induces proliferation in multiple PDX lines (FIG. 2K). Without wishing to be bound by theory, these data demonstrate that constitutive EGFR signaling mediates invasion while ligand-induced EGFR activation induces proliferation, providing support for the “go or grow” hypothesis.

Referring to FIGS. 2A-K and FIGS. 3A-N, EGFR signaling activity effects invasion of PDXs.

Specifically, FIG. 2A shows EGFR expression in multiple GBM PDXs by Western blotting. FIG. 2B shows the results of a Matrigel invasion assay in PDXs with or without EGF treatment. An equal number of cells (5×10⁴) were seeded on Matrigel coated inserts, and were untreated (control vehicle) or stimulated with 50 ng/ml EGF for 24 hours, cells invading to the other side of the membrane were counted and presented as percentage cell invasion. FIG. 2C shows the results of a Matrigel invasion assay of GBM12 treated with or without erlotinib (1 μM) for 24 or 48 hours. A similar invasion assay was conducted on GBM26 cells (FIG. 2D). FIG. 2E shows representative Western blotting of pEGFR and EGFR in cells after erlotinib treatment. FIG. 2F shows the results of a Matrigel invasion assay with EGFR siRNA knockdown in GBM12. Cells were transfected with EGFR or scrambled siRNA for 48 hours before the invasion assay. A similar invasion assay was performed on GBM6 (FIG. 2G). As shown in FIG. 2H, knockdown efficiency of EGFR siRNA was analyzed by Western blotting. FIG. 2I shows the results of a Matrigel invasion assay of GBM12 and GBM14 with EGFR overexpression. Cells were transiently transfected with empty or EGFR wildtype (EGFRwt) overexpressing vector, and invasion assay was performed after 48 hours after transfection. Overexpression of EGFR in GBM12 and GBM14 was confirmed by Western blotting (FIG. 2J). FIG. 2K shows the results of a Brdu incorporation assay of GBM12, GBM6, and GBM22. Cells were treated with or without 50 ng/ml EGF for 48 hours before the assay. The Western blot images are representative of three independent biological replicates. Actin served as loading control. Data are represented as mean±SEM from three independent experiments. *P<0.05, **P<0.01, n.s. not significant, unpaired two-tailed Student's t-test.

FIG. 3A shows the results of a Matrigel invasion assay of GBM28 and GBM120 treated with EGF (50 ng/ml) for 24 hors. Western blot analysis of EGFR in GBM28 and GBM120 is shown in FIG. 3B. FIG. 3C and FIG. 3D show the results of a Matrigel invasion assay of EGFR siRNA knockdown in GBM12 and GBM6 in response to EGF. Cells were treated with EGFR or scrambled siRNA for 48 hours, transfected cells were seeded on Matrigel coated inserts and were treated with or without 50 ng/ml EGF for 24 hours. Knockdown efficiency of EGFR was confirmed by Western blotting (FIG. 3E). FIG. 3F shows the results of a Matrigel invasion assay of GBM12 and GBM6 treated with low-dose EGF (5 ng/ml) for 24 hours. FIG. 3G shows the results of a Matrigel invasion assay of GBM12 and GBM6 treated with TGFα (20 ng/ml) for 24 hours. FIG. 3H shows the results of a Matrigel invasion assay of GBM12 and GBM6 treated with HB-EGF (20 ng/ml) for 24 hours. Results of a Matrigel invasion assay of GBM12 and GBM6 treated with 0.1 and 10 μM erlotinib for 24 and 48 hours are shown in FIG. 3I and FIG. 3J. The results of a Matrigel invasion assay of GBM12 treated with Cetuximab (100 μg/ml) or IgG for 24 hours are shown in FIG. 3K. FIG. 3L shows Western blot analysis of pEGFR, EGFR in GBM12 treated with Cetuximab or IgG in the presence or absence of EGF (50 ng/ml). FIG. 3M shows the results of a Matrigel invasion assay of EGFRvIII overexpressing GBM14. Cells were transiently transfected with EGFRvIII expression or empty vector, invasion assay was performed 48 hours after transfection. Overexpression of EGFRvIII in GBM14 was confirmed by Western blotting (FIG. 3N). Western blot images are representative of three independent biological replicates. Actin served as a loading control. Data are represented as mean±SEM from three independent experiments. **P<0.05, **P<0.01, ***P<0.001, n.s. not significant, unpaired two-tailed t-test.

Referring to FIG. 4A-D, an analysis of glioma cell migration by in vitro scratch assays is shown. Specifically, representative images of a GBM12 scratch assay in the absence and presence of 50 ng/ml EGF are shown in FIG. 4A. FIG. 4B shows a quantitative analysis of scratch wound closure. Representative images of a GBM6 scratch assay in the absence and presence of 50 ng/ml EGF are shown in FIG. 4C. FIG. 4D shows a quantitative analysis of scratch wound closure. Data are represented as mean±SEM from three independent experiments. ** P<0.01, unpaired two-tailed t-test.

Referring to FIG. 5A and FIG. 5B, data from a BrdU incorporation assay in neurospheres are shown. Specifically, the Brdu incorporation assay was done in neurospheres GBM9 and GBM429. Cells were incubated in neurosphere medium supplemented with 20 ng/ml FGF in the presence or absence of 20 ng/ml EGF for 2, 4, 6, or 8 days before the assay. Data are represented as mean±SEM from three independent experiments. *P<0.05, **P<0.01, *** P<0.001, unpaired two-tailed Student's t-test.

Referring to FIG. 6A-E, EGF inhibits EGFR overexpression induced cell invasion. Specifically, FIG. 6A shows the results of a Matrigel invasion assay of U251 cells stably transfected with the empty vector (U251V) or EGFR expression vector (U251EGFR) in response to EGF treatment (50 ng/ml). FIG. 6B shows the results of a Matrigel invasion assay of U87 cells stably expressing empty vector (U87V) or EGFR (U87EGFR) in response to EGF treatment (50 ng/ml). FIG. 6D shows the results of a Matrigel invasion assay of tetracycline induced EGFR overexpressing U251 cells (U251EGFRInd) in response to vehicle, tetracycline, or tetracycline plus EGF treatment. Tetracycline induced EGFR overexpression was confirmed by Western blotting (FIG. 6E). The Western blot images are representative of three independent biological replicates. Actin served as loading control. Data are represented as mean±SEM from three independent experiments. **P<0.01, ***P<0.001, n.s. not significant, unpaired two-tailed t-test.

3. Ligand-Activated EGFR Mediated Suppression of Invasion is Mediated by Upregulation of BIN3

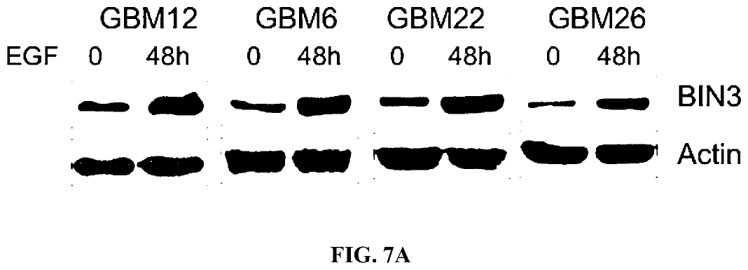

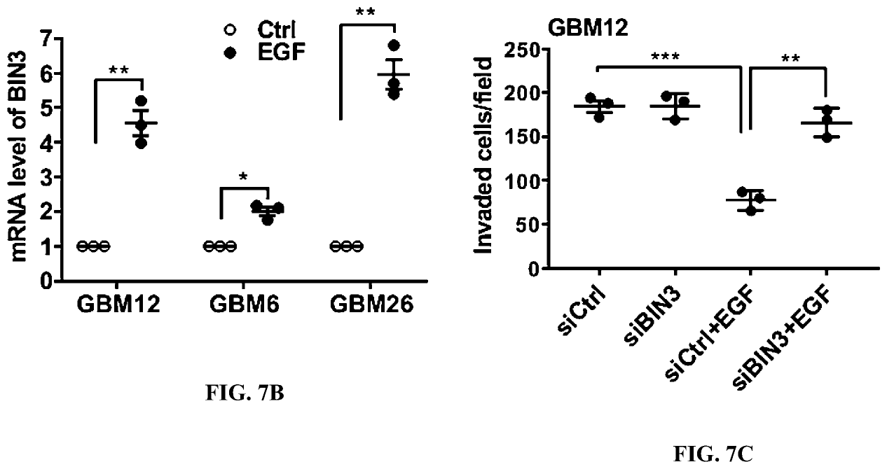

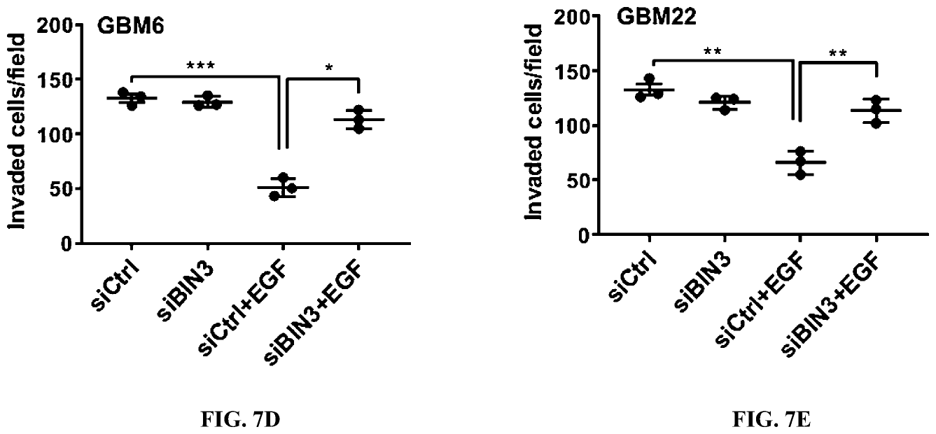

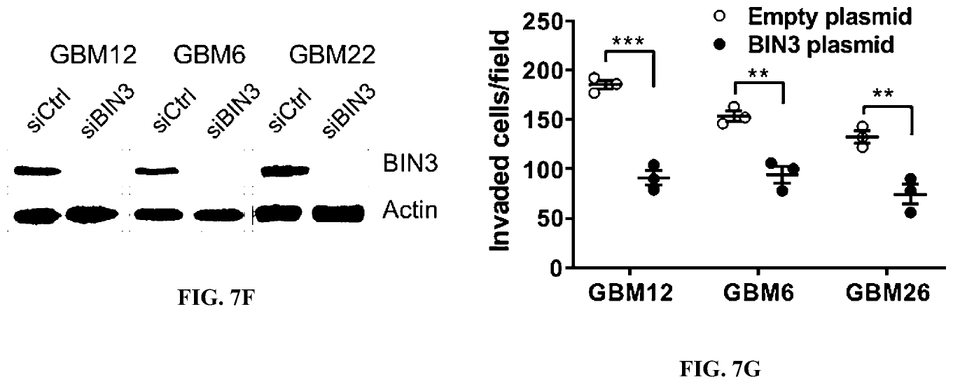

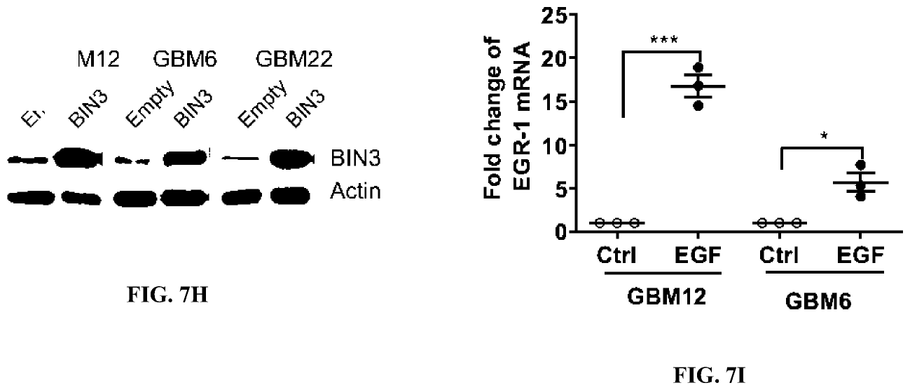

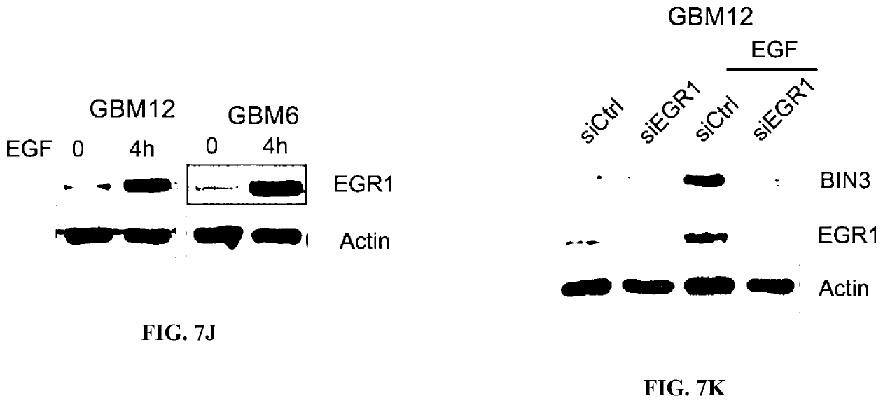

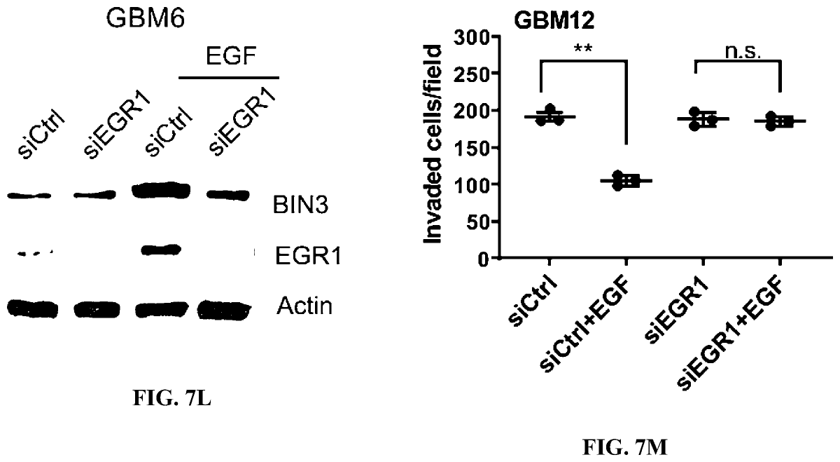

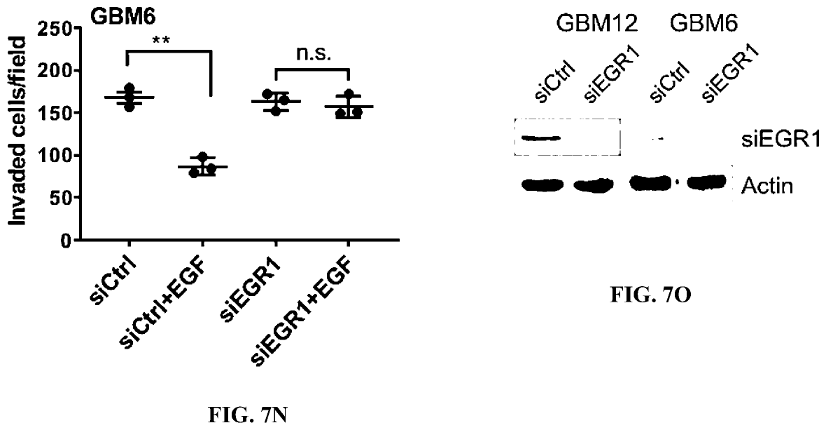

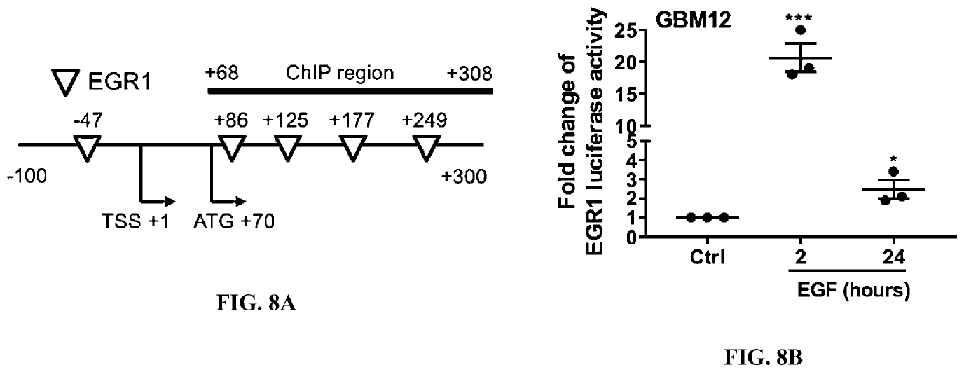

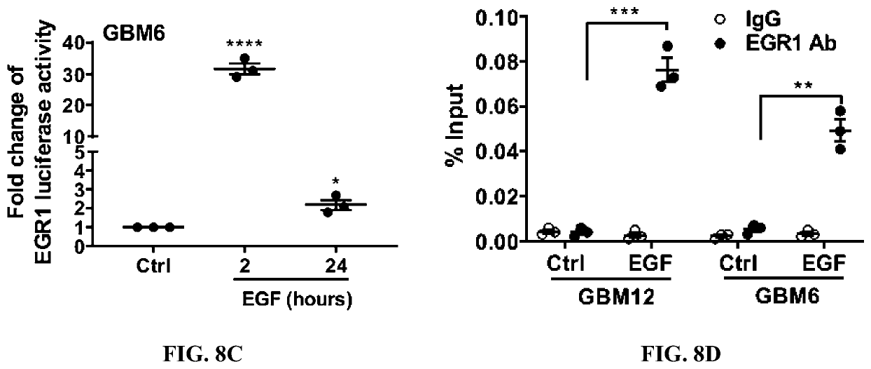

To uncover the downstream signaling mechanisms underlying the effect of EGFR on glioma cell invasion, RNA microarray data from a previous study was examined (Ramnarain et al. (2006) Cancer Res. 66: 867-874). It was found that 93 genes were upregulated by EGFR overexpression in glioma cells in the absence of exogenous EGF while 66 genes were upregulated only when EGF was added (Ramnarain et al. (2006) Cancer Res. 66: 867-874). A list of the genes upregulated by constitutive vs. ligand induced EGFR signaling is provided in Supplemental Table 1 of a previous study (Ramnarain et al. (2006) Cancer Res. 66: 867-874). It was hypothesized that ligand-induced activation of EGFR results in induction of specific downstream signals that inhibit glioma invasion. BIN3 was focused on because it is the most highly upregulated gene when EGF is added (19.2 fold) (Ramnarain et al. (2006) Cancer Res. 66: 867-874), and because of its known role in actin organization (Coll et al. (2007) EMBO J 26: 1865-1877). BIN3 is a member of the N-BAR domain family of proteins that may have a tumor suppressive function (Prendergast et al. (2009) Biochim. Biophys. Acta 1795: 25-36). BAR domains are involved in the regulation of membrane curvature (Peter et al. (2004) Science 303: 495-499), cell motility, and are also known to interact with small GTPases (Siminoescu-Bankston et al. (2013) Dev. Biol. 382: 160-171), which have a critical role in GBM invasion (Fortin Ensign et al. (2013) Front. Oncol. 3: 241). First, it was confirmed that addition of EGF to Mayo PDX lines leads to a robust increase in BIN3 by Western blot (FIG. 7A). In addition, consistent with a previous study, it was confirmed that BIN3 is regulated by EGF at the mRNA level (FIG. 7B). Importantly, it was found that siRNA knockdown of BIN3 rescued the ability of EGF to downregulate GBM invasiveness (FIG. 7C-F), up to here strongly supporting an essential role for BIN3 in EGF-mediated suppression of invasiveness. However, siRNA knockdown of BIN3 did not affect basal invasion in the absence of EGF, perhaps reflecting the low cellular level of BIN3 in the absence of EGF (which upregulates it), or a specific role for BIN3 in inhibiting invasion when it is upregulated. Overexpression of BIN3 results in decreased invasion (FIG. 7G and FIG. 7H). To identify the key transcription factor involved in EGFR mediated transcription of BIN3, results from a previous microarray experiment (Ramnarain, et al. (2006) Differential gene expression analysis reveals generation of an autocrine loop by a mutant epidermal growth factor receptor in glioma cells, Cancer Res 66: 867-874) were examined. EGR1 was identified as a highly induced (13.68 fold) gene that is not upregulated by constitutive EGFR signaling and upregulated only with ligand-activated EGFR signaling. EGR1 sites are present in the BIN3 promoter (FIG. 8A). The upregulation of EGR1 was confirmed by qPCR and by Western blot (FIG. 7I and FIG. 7J) and by reporter assays examining the activation of the transcriptional activity of EGR1 (FIG. 8B and FIG. 8C). Additionally, CHIP assays demonstrate that EGR1 occupies the BIN3 promoter when EGF is added (FIG. 8D). Importantly, siRNA knockdown of EGR1 blocked the ability of EGF to induce BIN3 (FIG. 7K and FIG. 7L) and also rescued EGF induced suppression of invasion (FIG. 7N and FIG. 7O).

Referring to FIG. 7A-0, EGF-mediated BIN3 upregulation inhibits invasiveness. Specifically, Western blot analysis of BIN3 in PDXs in response to EGF was performed (FIG. 4A). Cells were serum starved overnight and treated with 50 ng/ml EGF for 48 hours. BIN3 mRNA levels in EGF-treated PDXs were determined by real-time PCR (FIG. 7B). FIG. 7C shows the results of a Matrigel invasion assay of BIN3 siRNA knockdown GBM12 in response to EGF. Cells were treated with BIN3 or scrambled siRNA for 48 hours, transfected cells were treated with or without 50 ng/ml EGF for 24 hours. Similar experiments were conducted in GBM6 and GBM22 (FIG. 7D and FIG. 7E). Referring to FIG. 7F, knockdown efficiency of BIN3 siRNA was analyzed by Western blotting. FIG. 7G shows the results of a Matrigel invasion assay of PDXs with BIN3 overexpression. Cells were transiently transfected with vector expressing BIN3 for 48 hours followed by invasion assay. BIN3 overexpression in GBM12, GBM6, and GBM26 was confirmed by Western blotting (FIG. 7H). EGR1 mRNA levels in GBM12 and GBM6 after EGF treatment were determined by real-time PCR (RT-PCR) (FIG. 7I). FIG. 7J shows the results of Western blot analysis of EGR1 in GBM12 and GBM6 treated with EGF for 4 hours. FIG. 7K shows the results of Western blot analysis of BIN3 in EGR1 siRNA knockdown GBM12 in the absence and presence of EGF (50 ng/ml). A similar experiment was performed in GBM6 (FIG. 7L). FIG. 7M shows the results of a Matrigel invasion assay of EGR1 siRNA knockdown GBM12 in the absence and presence of EGF (50 ng/ml). A similar experiment was performed in GBM6 (FIG. 7N). Knockdown efficiency of EGR-1 siRNA was analyzed by Western blotting (FIG. 7O). The Western blot images are representative of three independent biological replicates. Actin served as loading control. Data are represented as mean±SEM from three independent experiments. * P<0.05, **P<0.01, unpaired two-tailed t-test.

Referring to FIG. 8A-D, EGF induces EGR1 activity and enrichment on the BIN3 promoter. Specifically, FIG. 8A shows a schematic diagram of the putative EGR binding sites in BIN3 promoter region together with the corresponding ChIP-qPCR amplicons. FIG. 8B shows EGR1 luciferase reporter activity in response to EGF (50 ng/ml) in GBM12. Cells were transiently transfected with EGR1 firefly luciferase reporter vector and Renilla luciferase vector for 48 hours, EGF was added for additional 2 or 24 hours prior to measuring luciferase activity. FIG. 8C shows EGR1 luciferase reporter activity in response to EGF in GBM6. FIG. 8D shows the percentage input done by ChIP-qPCR to assess the EGR-1 occupancy of BIN3 gene in EGF treated GBM12 and GBM6. Cells were treated with or without EGF for 24 hours. ChIP assay was performed as described elsewhere herein. IgG was used as negative control. Data are represented as mean±SEM from three independent experiments. *P<0.05, **P<0.01, ***P<0.001, ****P<0.0001, unpaired two-tailed t-test.

4. BIN3 Downregulates Invasion Via Interaction with DOCK7 and Inhibition of Rho GTPases

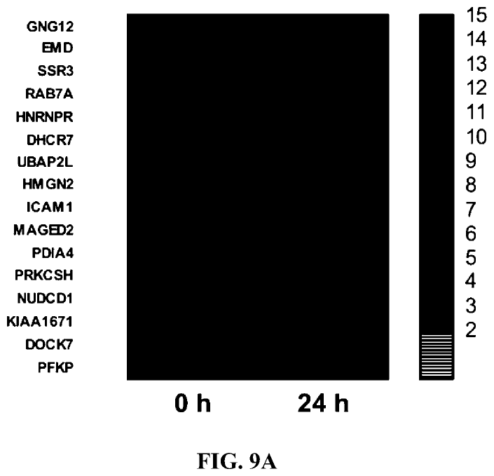

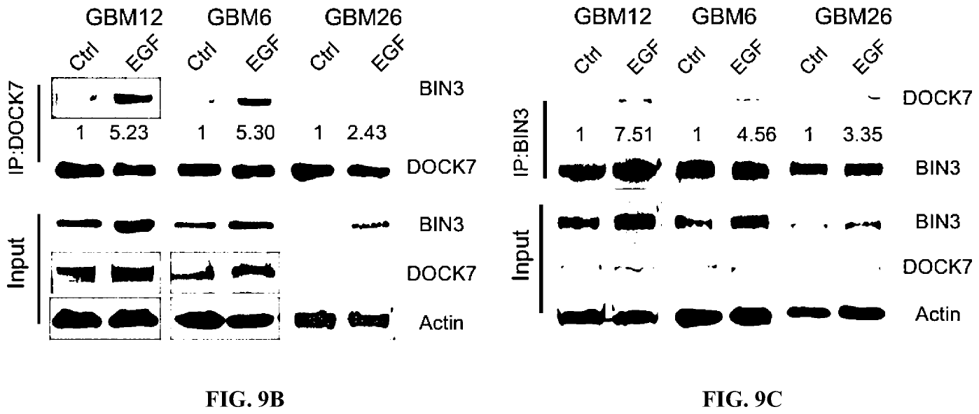

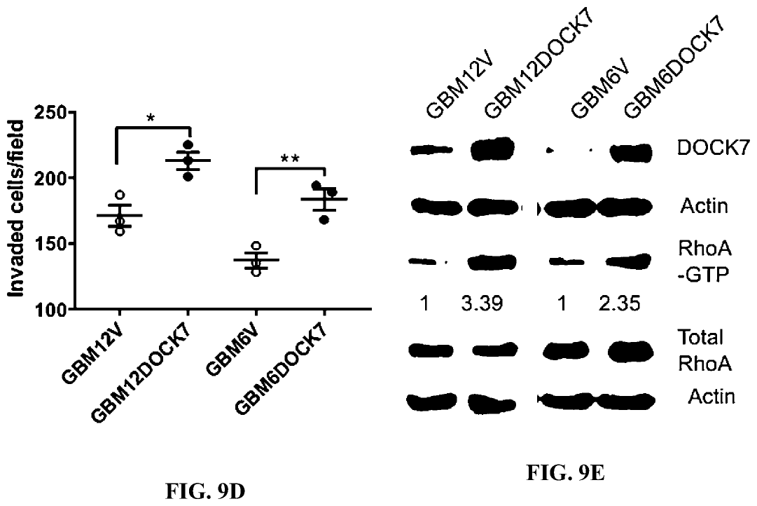

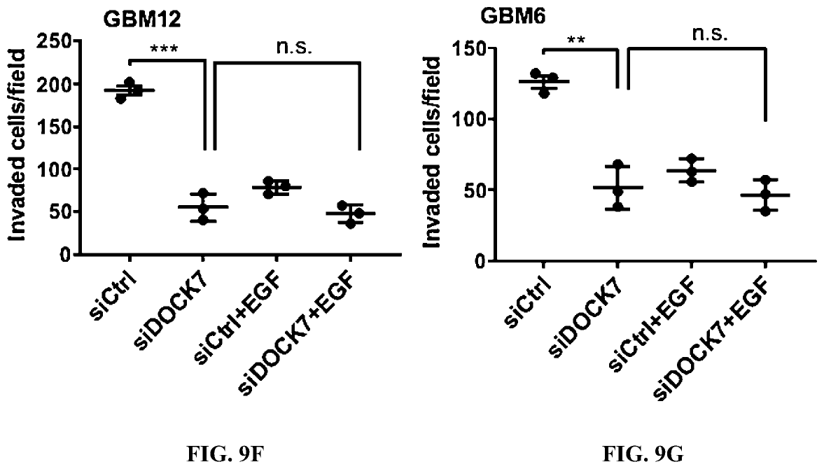

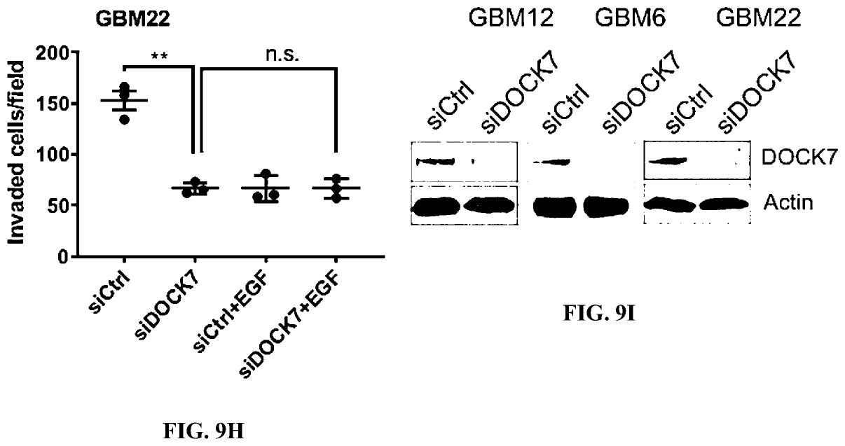

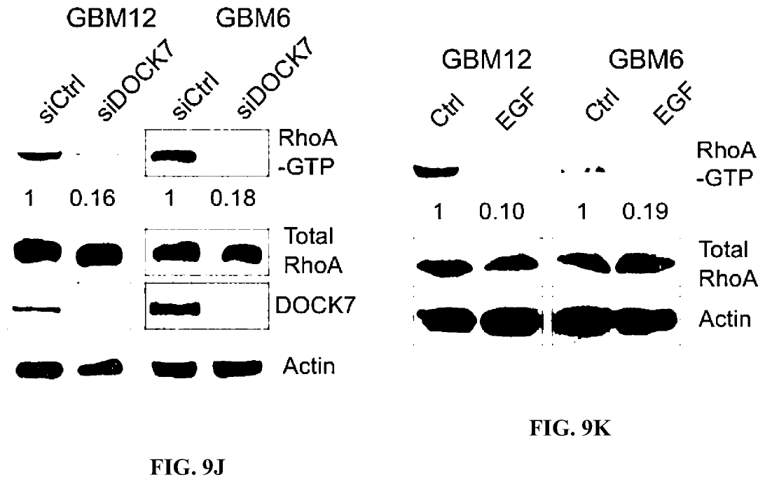

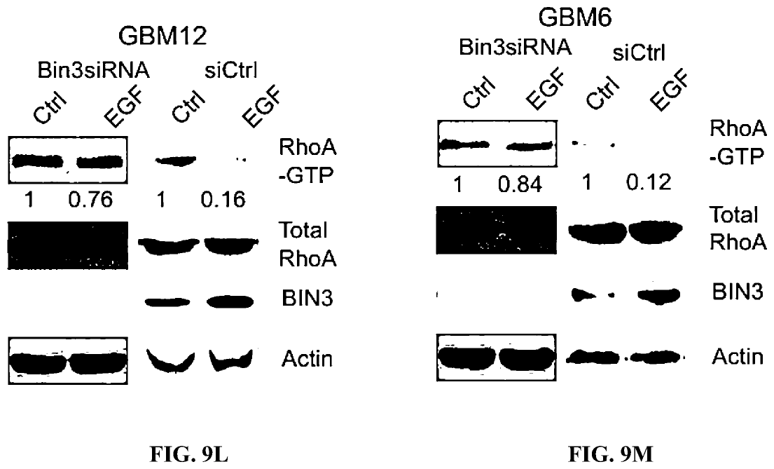

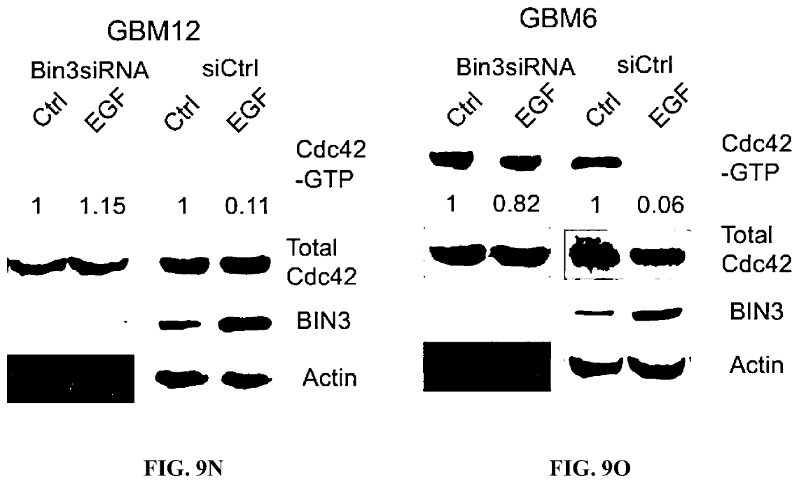

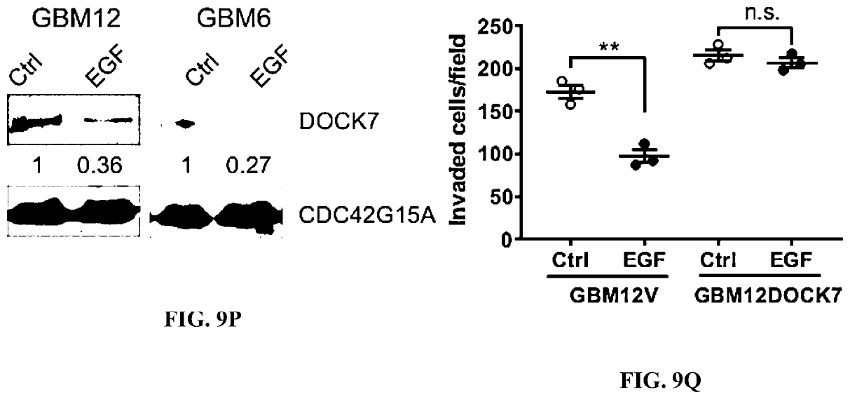

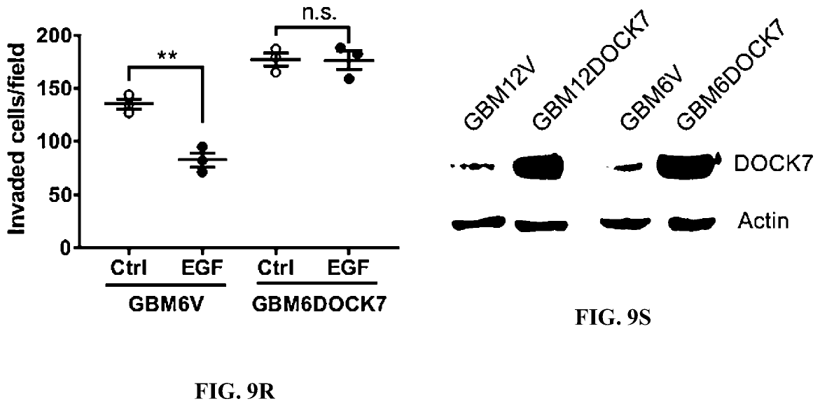

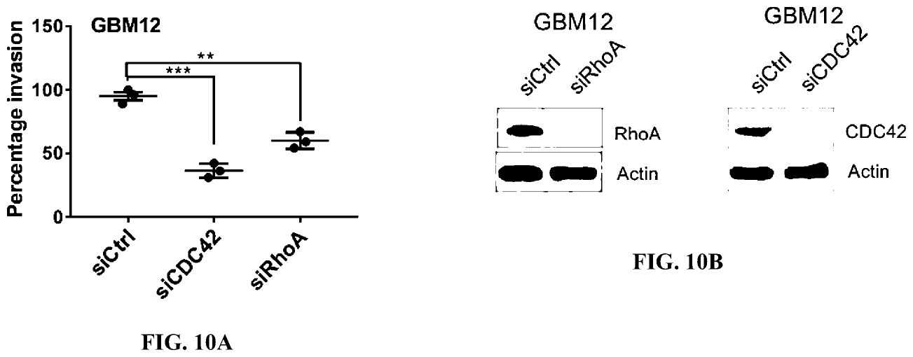

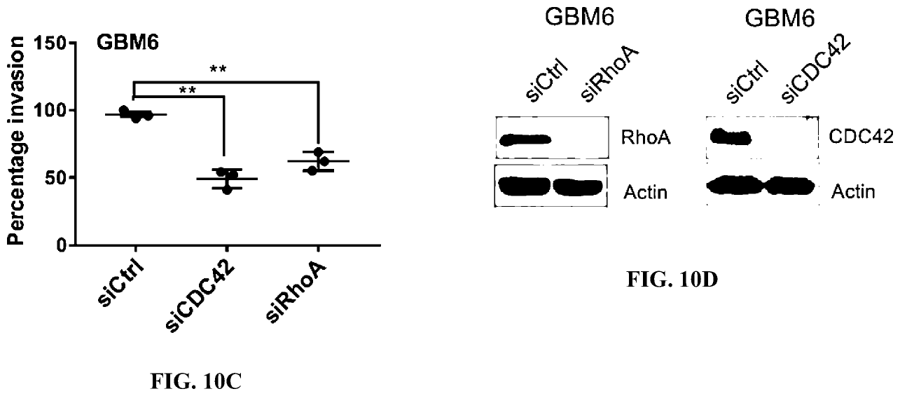

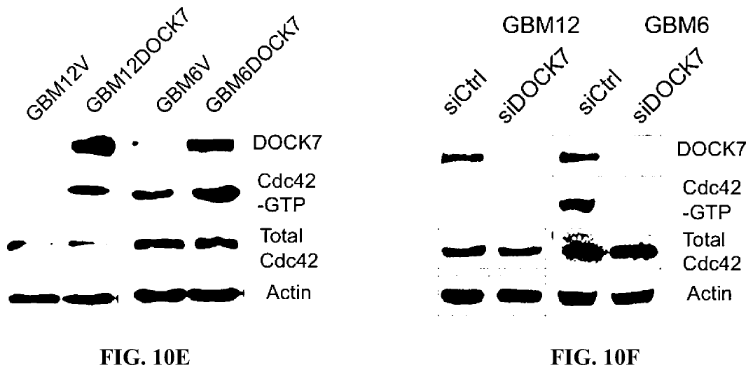

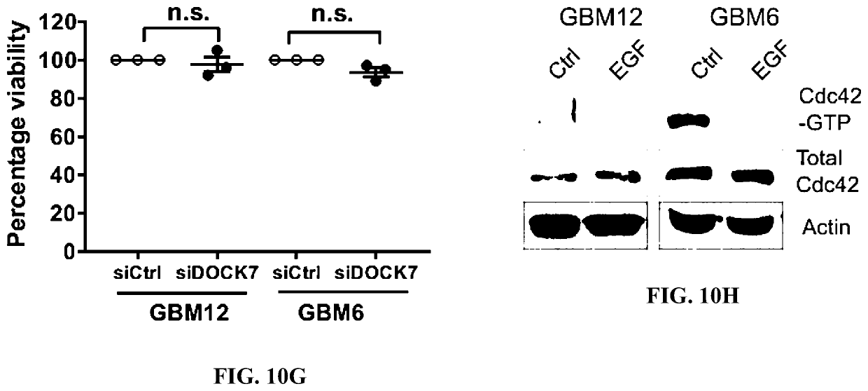

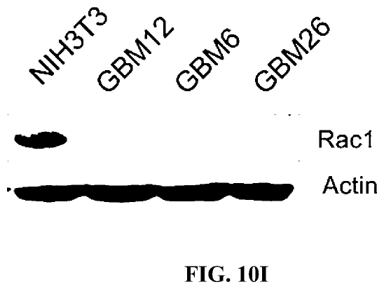

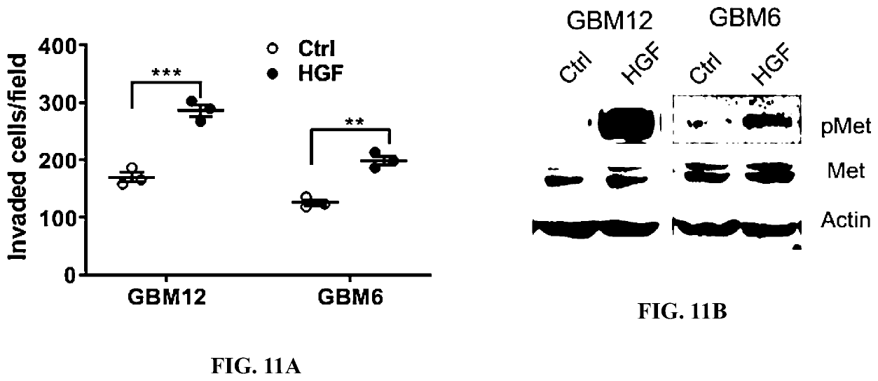

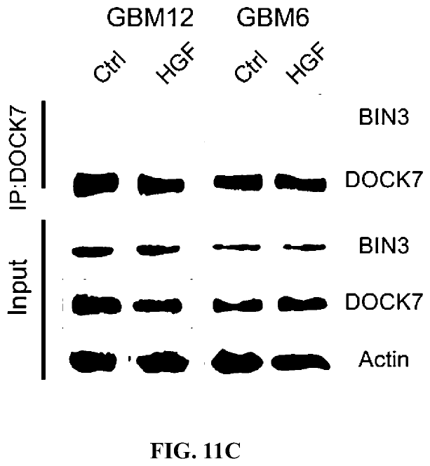

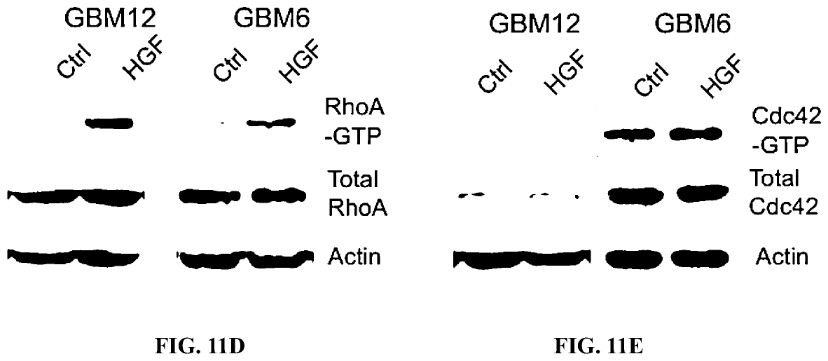

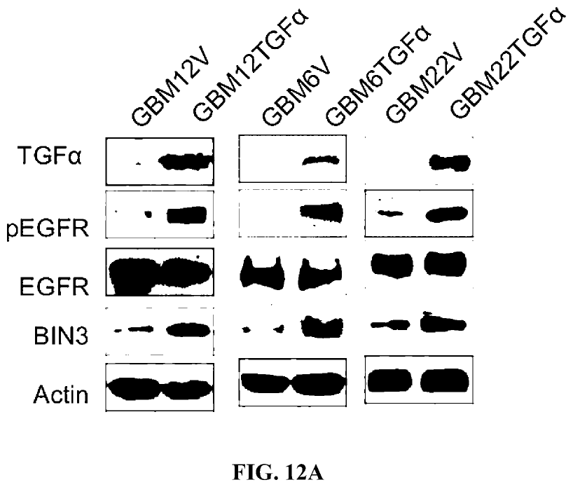

To elucidate the mechanism of action of BIN3, mass spectrometry was undertaken to identify proteins associating with BIN3. This analysis was undertaken in Mayo PDX GBM12 explant cultures. 12 proteins that associate with BIN3 only in the presence of EGF were identified, including DHCR7, DOCK7, EMD, RAB7A, and ICAM1 (FIG. 9A). An investigation of DOCK7 was prioritized based on a previously described role in cell motility/invasion. DOCK7 (dedicator of cytokinesis 7): DOCK7 is a member of the DOCK180 family of atypical Rac/Cdc42 guanine nucleotide exchange factors (GEFs) (Gadea and Blangy (2014) Eur. J. Cell Biol. 93: 466-477). DOCK7 has been implicated in neuronal precursor migration (Nakamuta et al. (2017) J. Cell Biol. 216: 4313-4330), and has been reported to mediate HGF-induced glioblastoma cell invasion via Rac activation (Murray et al. (2014) Br. J. Cancer 110: 1307-1315; Yamamoto et al. (2013) Oncology reports 29: 1073-1079). Previous work suggests that DOCK7 has a pro-oncogenic and invasive effect. First, it was confirmed that EGF significantly induced a physical association between DOCK7 and BIN3 in co-immunoprecipitation experiments using a DOCK7 antibody for immunoprecipitation followed by Western blot with BIN3 antibody (FIG. 9B). A reciprocal experiment with BIN3 antibody used for immunoprecipitation and a DOCK7 antibody for Western blot gave the same result (FIG. 9C). It was hypothesized that DOCK7 constitutively activates Rho GTPases and drives invasion and that the association of BIN3 with DOCK7 inhibits the function of DOCK7. To examine this hypothesis, it was first confirmed that Rho GTPases play a key role in invasion by demonstrating that siRNA knockdown of RhoA and CDC42 leads to decreased invasiveness in transwell invasion assays (FIG. 10A-D). Next, it was found that increased expression of DOCK7 results in increased invasiveness and increased activity of Rho GTPases (FIG. 9D, FIG. 9E, and FIG. 10E). Conversely, siRNA knockdown of DOCK7 results in decreased invasiveness and decreased activity of Rho GTPases (FIGS. 9F-J and FIG. 10F). Addition of EGF to cells with siRNA knockdown of DOCK7 did not decrease invasiveness further indicating that DOCK7 is downstream of BIN3 (FIG. 9F-H). siRNA knockdown of DOCK7 has no effect on viability of cells (FIG. 10G). Next, it was demonstrated that EGF decreases the activity of both RhoA and CDC42 (FIG. 9K and FIG. 10H) Rac activity was not assessed because Rac levels are low in these cells (FIG. 10I). Importantly, siRNA knockdown of BIN3 results in a loss of the EGF-mediated downregulation of Rho GTPase activity, confirming that BIN3 is required for EGF mediated downregulation of Rho GTPases (FIG. 9L-O). Next, it was confirmed that exposure of cells to EGF results in decreased activity of DOCK7 (FIG. 9P) . . . . Overexpression of DOCK7 rescued the EGF effect on invasion (FIG. 9Q-S). As an additional control, the effect of HGF, the ligand for Met, was also tested. Unlike EGF, HGF increased invasiveness of glioma cells and Met phosphorylation (FIG. 11A and FIG. 11B). Furthermore, an association between BIN3 and DOCK7 was not detected in response to HGF (FIG. 11C), and addition of HGF resulted in an increase in RhoA activity, although an increase of CDC42 activity was not detected (FIG. 11D and FIG. 11E). Consistent with these data it was also find that HGF does not affect BIN3 levels (FIG. 11C). Without wishing to be bound by theory, these data indicate the specificity of the BIN3-DOCK7 interaction in response to EGF and support a model in which ligand mediated activation of the EGFR results in decreased invasion via a BIN3 mediated inhibition of a DOCK7-Rho GTPase pathway.

Referring to FIG. 9A-S, BIN3 reduces invasion by interacting with DOCK7. Heat map of spectral counts of the top BIN3 interacting proteins identified by mass spectrometry in GBM12 treated with EGF (50 ng/ml) is shown in FIG. 9A. Western blot analysis of immunoprecipitated extracts from EGF-treated GBM12, GBM6, and GBM26 is illustrated in FIG. 9B. Cell lysates from cells treated with EGF (50 ng/ml) or control vehicle for 24 hours were immunoprecipitated by DOCK7 antibody, followed by Western blot for BIN3. Western blot analysis of BIN3 antibody immunoprecipitated extracts from GBM12, GBM6, and GBM26 24 hours after EGF or vehicle treatment is illustrated in FIG. 9C. Cell lysates from cells treated with EGF (50 ng/ml) or control vehicle for 24 hours were immunoprecipitated by BIN3 antibody, followed by Western blot for DOCK7. FIG. 9D shows the results of a Matrigel invasion assay of DOCK7 overexpressing GBM12 and GBM6. Cells were infected with DOCK7 or scrambled shRNA virus particles for 48 hours, transfected cells were seeded on Matrigel coated inserts in the absence or presence of EGF (50 ng/ml) for 24 hours. Western blotting showing GTP-bound RhoA (RhoA-GTP) and total RhoA levels in DOCK7 overexpressing GBM12 and GBM6, which were generated as described above, is shown in FIG. 9E. FIG. 9F shows the results of a Matrigel invasion assay of DOCK7 siRNA knockdown GBM12 in the absence of presence of EGF (50 ng/ml) for 24 hours. Similar experiments were conducted in GBM6 and GBM22 (FIG. 9G and FIG. 5H). DOCK7 siRNA knockdown was confirmed by Western blot analysis (FIG. 9FI. FIG. 9J shows a Western blot illustrating Rhoa-GTP and total RhoA levels in DOCK7 siRNA knockdown GBM12 and GBM6. FIG. 5K shows a Western blot illustrating GTP-bound RhoA (RhoA-GTP) and total RhoA levels in GBM12 and GBM6 treated with EGF (50 ng/ml) for 24 hours. FIG. 9L and FIG. 9M show Western blotting illustrating Rhoa_GTP in BIN3 siRNA knockdown GBM12 and GBM6 in the presence or absence of EGF (50 ng/ml) for 24 hours. FIG. 9N and FIG. 9O show Western blotting illustrating CDC42-GTP in BIN3 siRNA knockdown GBM12 amd GBM6 in the presence and absence of EGF (50 ng/ml) for 24 hours. FIG. 9P shows a Western blot illustrating active DOCK7 expression in EGF-treated GBM12 and GBM6. Cells were treated with EGF for 24 hours, cell lysates were incubated with agrose CDC42G15A to pull down active DOCK7. FIG. 9Q shows the results of a Matrigel invasion assay of DOCK7 overexpressing GBM12 in response to EGF (50 ng/ml). GBM12 were treated with DOCK7 shRNA or control virus particles for 48 hours before the invasion assay. Similar experiments were performed in GBM6 (FIG. 9O). Overexpression of DOCK7 in GBM12 and GBM14 was confirmed by Western blotting (FIG. 9P). The numbers under the gel lanes represent the relative protein level, which was normalized to Actin. The Western blot images are representative of three independent biological replicates. Actin served as loading control. Data are represented as mean±SEM from three independent experiments. **P<0.01, ***P<0.001, n.s. not significant, unpaired two-tailed t-test.

Referring to FIG. 10A-I, siRNA-mediated knockdown of CDC42 or RhoA results in reduced invasiveness. Specifically, FIG. 10A shows the results of a Matrigel invasion assay of CDC42 or RhoA siRNA knockdown in GBM12. Cells were transfected with CDC42, RhoA, or control siRNA transfection for 48 hours, transfected cells were seeded on Matrigel coated inserts for 24 hours. Knockdown efficiency of RhoA and CDC42 siRNA was analyzed by Western blot (FIG. 10B). FIG. 10C shows the results of a Matrigel invasion assay of CDC42 or RhoA siRNA knockdown GBM6. Knockdown efficiency of RhoA and CDC42 siRNA was analyzed by Western blot (FIG. 10D). A Western blot showing CDC42-GTP and total CDC42 levels in DOCK7 overexpressing GBM12 and GBM6 is shown in FIG. 10E. Cells with overexpressed DOCK7 were generated as described for FIG. 9A. A Western blot showing CDC42-GTP in GBM12 and GBM6 following with DOCK7 siRNA transfection is shown in FIG. 10F. FIG. 10G shows the percentage of cell viability in DOCK7 or scrambled siRNA for 48 hours, transfected cells were seeded on 96 cell well plate for additional 48 hours before MTT assay. Western blot analysis showing GTP-bound CDC42 (CDC42-GTP) and total CDC42 levels in GBM12 and GBM6 treated with EGF (50 ng/ml) for 24 hours is shown in FIG. 10H. Western blot analysis of Rac1 in multiple PDXs is shown in FIG. 10I. The Western blot images are representative of three independent biological replicates. Actin served as a loading control. Data are represented as mean±SEM from three independent experiments. ** P<0.01, n.s. not significant, unpaired two-tailed t-test.

Referring to FIG. 11A-E, HGF induces invasion in GBM12 and GBM6. Specifically, FIG. 11A shows the results of a Matrigel invasion assay of GBM12 and GBM6 in response to HGF. Cells were seeded on Matrigel coated inserts for 24 hours in the absence or presence of HGF (20 ng/ml). A Western blot showing pMet and total Met in GBM12 and GBM6 treated with HGF (20 ng/ml) is shown in FIG. 11B. A Western blot analysis of DOCK7 antibody immunoprecipitated extracts from GBM12 and GBM6 treated with HGF for 24 hours (20 ng/ml) is shown in FIG. 11C. A Western blot showing GTP-bound RhoA (RhoA-GTP) and total RhoA levels after HGF treatment for 24 hours in GBM12 and GBM6 is shown in FIG. 11D. A Western blot showing CDC42-GTP and total CDC42 levels after HGF treatment (20 ng/ml) for 24 hours in GBM12 and GBM6. The Western blot images are representative of three independent biological replicates. Actin served as loading control. Data are represented as mean±SEM from three independent experiments. *** P<0.001, **** P<0.0001, unpaired two-tailed t-test.

5. Effects of Constitutive Vs. Ligand-Activated EGFR Signaling In Vivo

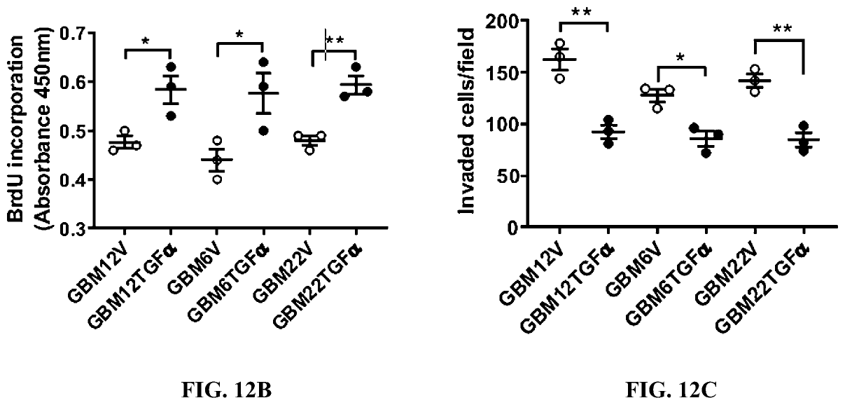

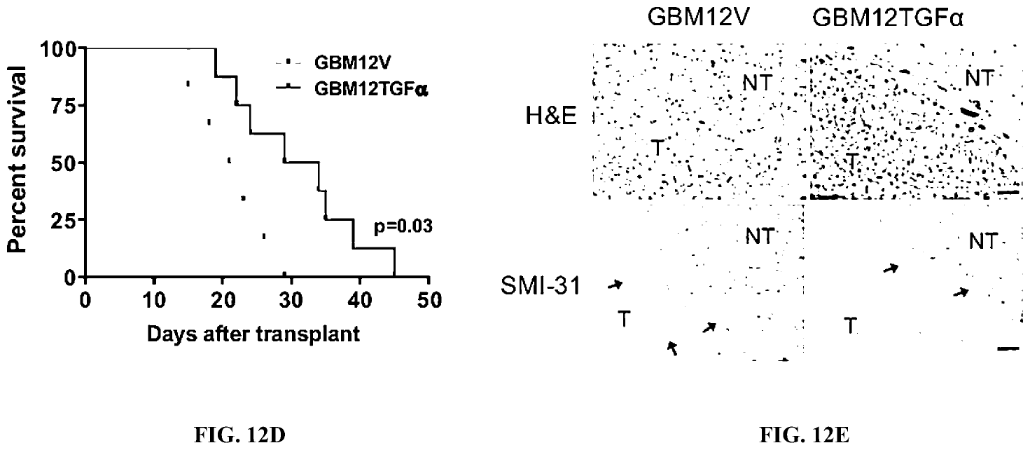

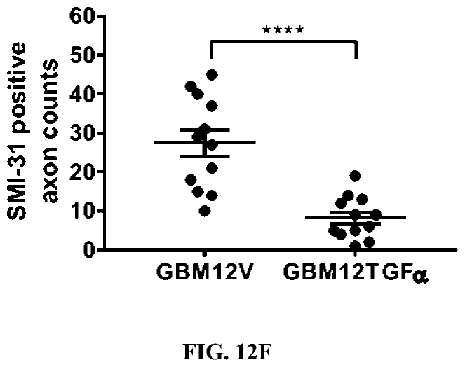

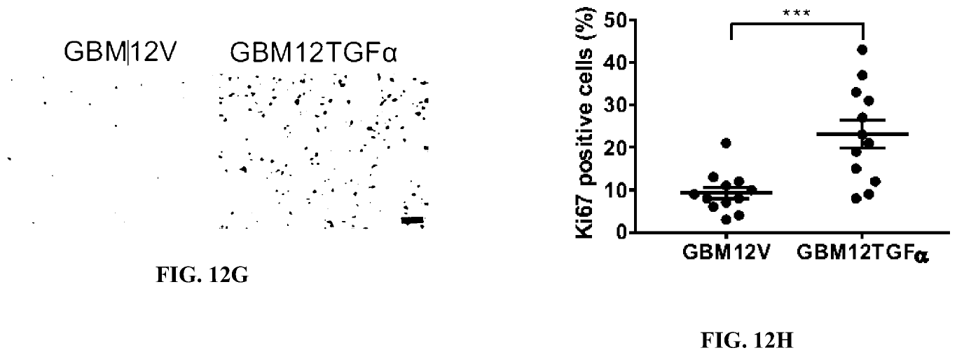

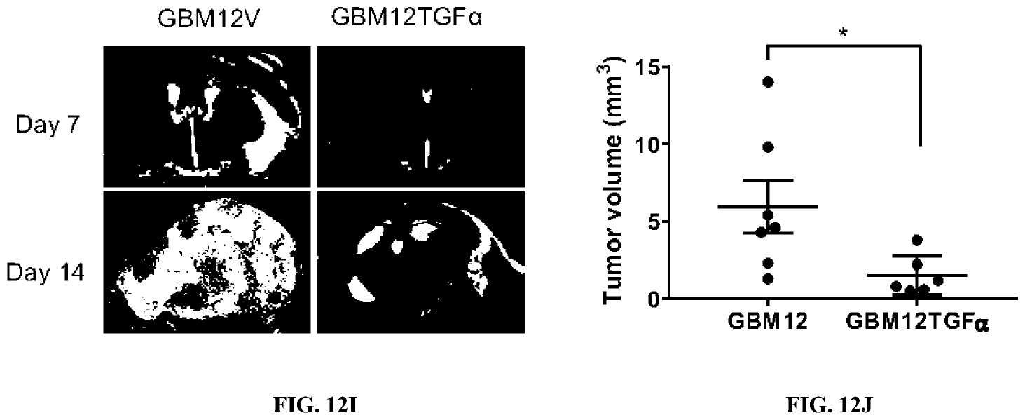

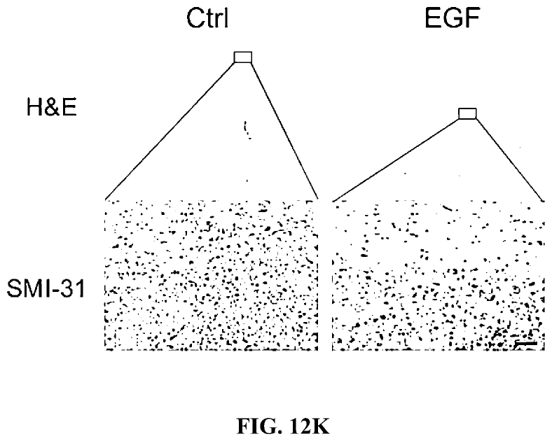

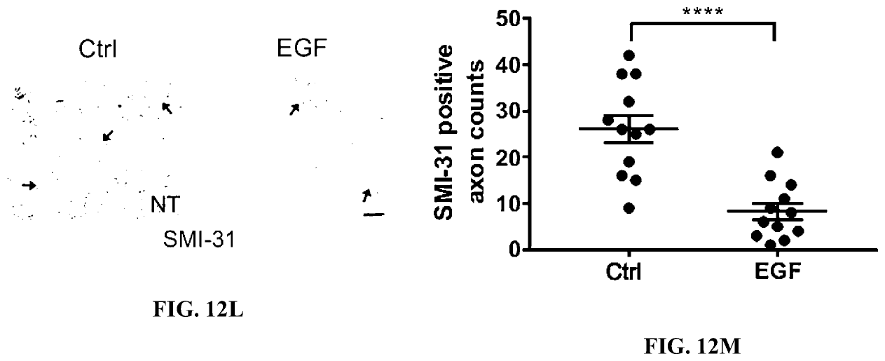

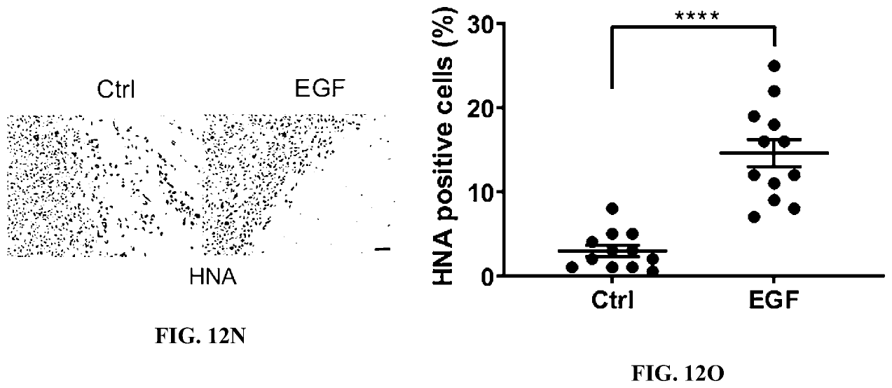

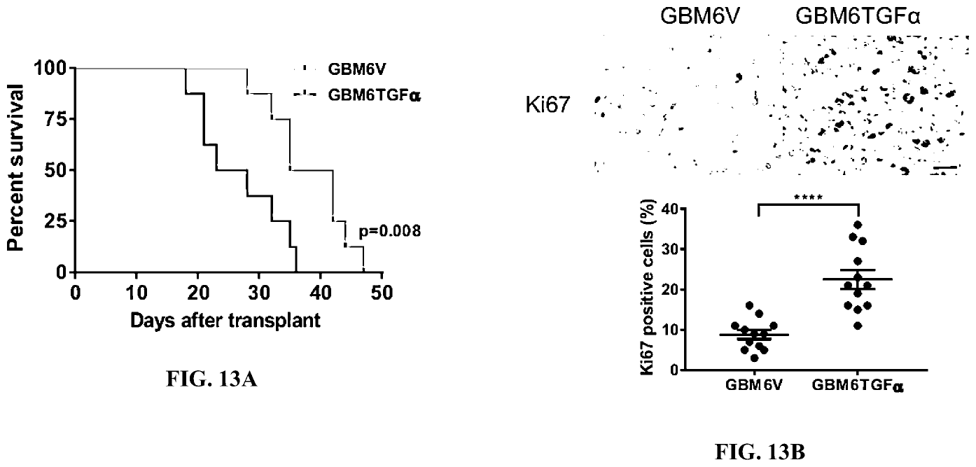

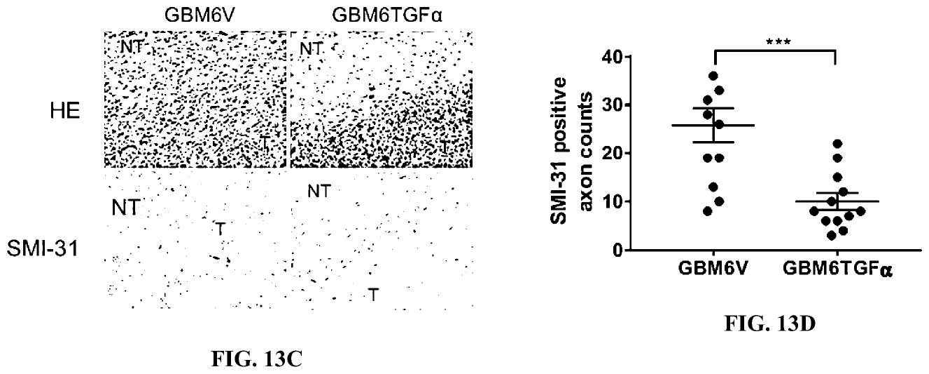

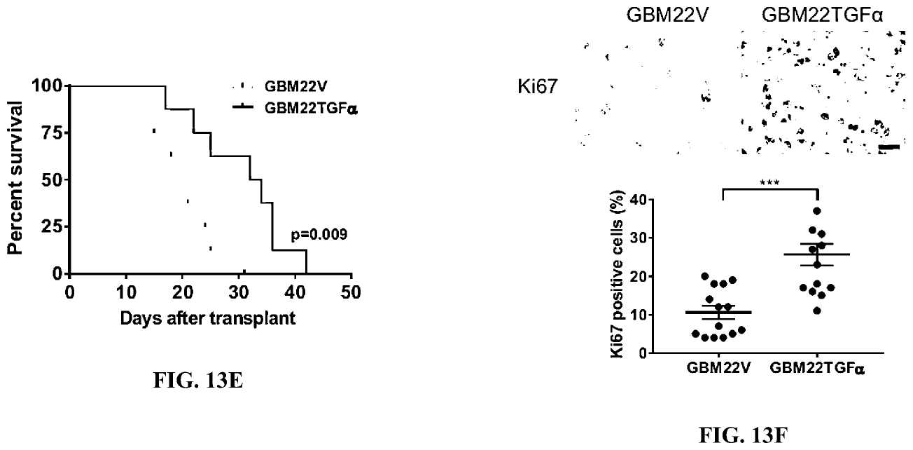

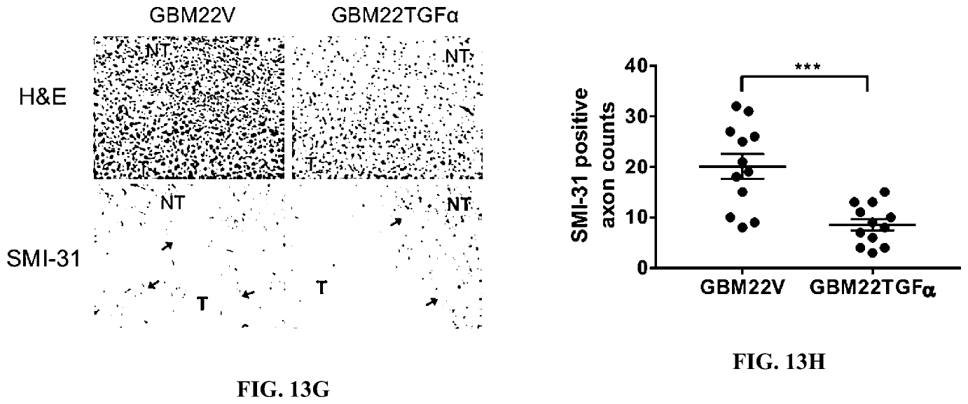

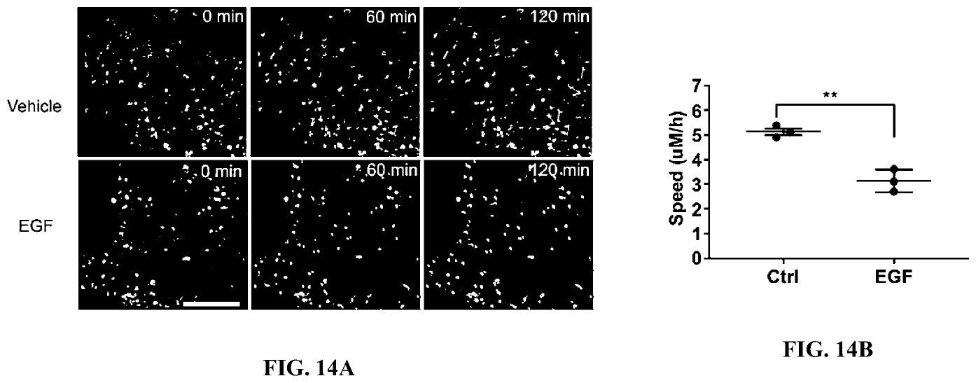

Next, the impact of EGFR ligand on GBM invasion was examined in an orthotopic mouse model. To induce ligand activation of EGFR in vivo, ligand to EGFR-expressing tumors were provided by generating an autocrine loop or by exogenous infusion of ligand. In the autocrine loop experiment, ligand for EGFRwt is provided by the same cells that express the EGFR. Autocrine loops involving EGFR and its ligands are well described in GBM (Ramnarain et al. (2006) Cancer Res. 66: 867-874; Tang et al. (1997) J. Neurooncol. 35: 303-314). Mayo PDX explant cultures were stably transfected with TGFα (FIG. 12A), since a TGFα autocrine loop has been described previously in GBM (Tang et al. (1997) J. Neurooncol. 35: 303-314). Increased TFGα expression resulted in tyrosine phosphorylation of the EGFR and upregulation of BIN3 confirming that the expressed ligand is functional (FIG. 12A). Increased expression of TGFα resulted in increased proliferation and decreased invasion in ex vivo assays consistent with results obtained by adding exogenous ligand (FIG. 12B and FIG. 12C). These data were observed for both EGFRwt expressing and EGFRvIII+EGFRwt expressing PDXs. Next, TGFα transfected GBM12 cells were injected intracranially in athymic mice, followed by a survival analysis. It was found that increased ligand availability results in improved survival (FIG. 12D) and a sharp decrease in invasiveness (FIG. 12E and FIG. 12F), even though proliferation is increased in TGFα expressing tumors (FIG. 12G and FIG. 12H). Similar results were found for GBM6 expressing both EGFRwt plus EGFRvIII and for EGFRwt expressing GBM22 with TGFα expression resulting in hyperproliferative and non-invasive tumors with a better prognosis (FIG. 13A-H). To monitor the effect of TGFα expression on the size of the tumor, an additional experiment was undertaken using GBM12TGFα expressing cells, and serial MRI imaging was done (FIG. 12I and FIG. 12J). While the tumor size is similar at 7 days, at 14 days a striking difference in the size of the tumors is detectable with TGFα expression resulting in much smaller tumors. A 2^{nd} TGFalpha expressing GBM12 line also demonstrated similar results. The effect of exogenous EGF infusion into GBM12 mouse intracranial tumors was also examined. EGF was infused directly into the lateral ventricles using a miniosmotic pump attached to a cannula for 4 weeks as described previously (Bachoo et al. (2002) Cancer Cell 1: 269-277). MRI imaging was done every week for 4 weeks after which mice were euthanized. Consistent with the results of the autocrine loop experiment, exogenous infusion of EGF also resulted in smaller tumors that were noninvasive (FIG. 12K-M). A decreased invasiveness can also be demonstrated by IHC staining for a human nuclear marker (FIG. 12N-O). Additionally, intravital microscopy was used to monitor the motility of GBM cells in the live mouse brain in response to EGF. GBM12 cells were injected into mouse brain. A cranial window was placed and EGF was injected through it to monitor the movement of glioma cells through the brain. While significant migration of glioma cells can be detected following injection of control vehicle, injection of EGF results in an almost complete cessation of movement (FIG. 14A and FIG. 14B). Without wishing to be bound by theory, this experiment provides additional confirmation of the suppressive effect of EGF on glioma invasiveness.

Referring to FIG. 12A-O, ligand induced EGFR signaling inhibits invasion of PDXs. Western blot analysis of TGFα, pEGFR, EGFR, and BIN3 in PDXs stably transfected with empty (GBM12V, GBM6V, GBM22V) or TGFα expressing vector (GBM12TGFα, GBM6TGFα, GBM22TGFα) is shown in FIG. 12A. The Western blot images are representative of three independent biological replicates. Actin served as loading control. FIG. 12B shows the results of a BrdU incorporation assay of the TGFα-overexpressing PDXs as described in panel A. FIG. 12C shows the results of a Matrigel invasion assay of the TGFα overexpressing PDXs as described in panel A. Kaplan-Meier survival curves of mice with orthotopic xenotransplant model of GBM12V and GBM12TGFα, n=8 each group, are shown in FIG. 12D. Representative H&E staining and immunostaining for SMI-31 in GBM12V and GBM12TGFα tumor tissue sections is shown in FIG. 12E. Quantification analysis of SMI-31 n GBM12V and GBM12TGFα tumor tissue sections is shown in FIG. 12F. FIG. 12G shows representative images of Ki67 immunostaining in mice tumor tissue sections. FIG. 12H shows a quantification analysis of Ki67 stained mouse tumor sections. Representative MRI imaging of orthotic tumor bearing mice obtained at 7 and 14 days after transplantation of GBM12V or GBM12TGFα is shown in FIG. 12I. Tumor volume of two groups after 14 days (GBM12V n=7, GBM12TGFα n=6) is shown in FIG. 12J. FIG. 12K shows H&E staining of GBM12 orthtopic tumors from mice treated with or without EGF. GBM12 bearing mice were intracranially infused with EGF or vehicle (Control) continually for 2 weeks. Mice were then sacrified and tumor samples were collected for H&E and immunostaining staining for SMI31 (n=4 each group). FIG. 11L shows representative immunostaining for SMI-31 in GBM12 orthotopic tumors from mice (n=4 each group) treated with or without EGF. Quantification of SMI-31 counts in mouse tumor tissue sections is shown in FIG. 11M. FIG. 11N shows representative immunostaining for anti human nuclear Antigen antibody (HNA) in GBM12 orthotopic tumors from mice (n=4 each group) treated with or without EGF. Quantitative analysis of HNA positive cells in mouse tumor tissue sections. Scale bars: 25 μM. Data are represented as mean±SEM from three independent experiments. *P<0.05, ****P<0.0001, unpaired two-tailed t-test.

Referring to FIG. 13A-H, TGFα overexpression prolongs survival, reduces invasiveness, and increases proliferation in an orthotopic glioblastoma mouse model. Kaplan-Meier survival curves of mice with orthotopic xenotransplant model of GBM6 stably transfected with empty vector (GBM6V) and TGFα overexpression vector (GBM6TGFα), n=8 each group, are shown in FIG. 13A. FIG. 13B shows representative images and quantification of Ki67 staining in GBM6V and GBM6TGFα tumor tissue sections. FIG. 13C shows H&E staining and immunostaining of SMI-31 in GBM6V and GBM6TGFα tumor tissue sections. Quantification of SMI-31 counts in mouse tumor tissue sections is shown in FIG. 13D. Kaplan-Meier survival curves of mice with orthotopic xenotransplant model of control GBM22V and GBM22TGFα tumor tissue sections are shown in FIG. 13E. FIG. 13F shows representative images and quantification of Ki67 staining of GBM22V and GBM22TGFα tumor tissue sections. FIG. 13G shows H&E staining and immunostaining of SMI31 in GBM22V and GBM22TGFα tumor tissue sections. Quantification of SMI-31 counts in tumor tissue sections is shown in FIG. 13H. Data are represented as mean±SEM from three independent experiments. *** P<0.001, **** P<0.0001, unpaired two-tailed t-test.

Referring to FIG. 14A and FIG. 14B, EGF reduces cell migration speed in vivo detected by intravital microscopy. FIG. 14A shows representative time-lapse imaging of migrating tumor cells over 120 minutes through a cranial window. Red lines highlight individual tumor cell tracks. Scale bar: 100 μm. FIG. 14B shows representative quantification of cell velocity for vehicle (Ctrl) and EGF treated group (n=3 mice each group). The cell velocity of around 200 cells per mouse were evaluated. Each point represents the average cell velocity of the total cells per mouse. The data is shown as mean±S.E.M. ** P<0.01, unpaired two-tailed t-test.

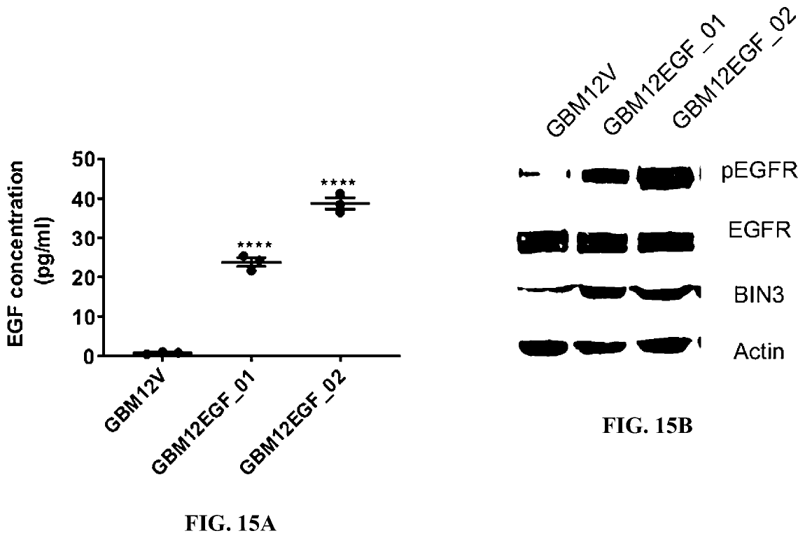

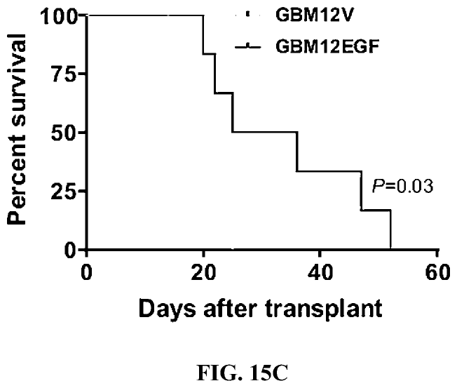

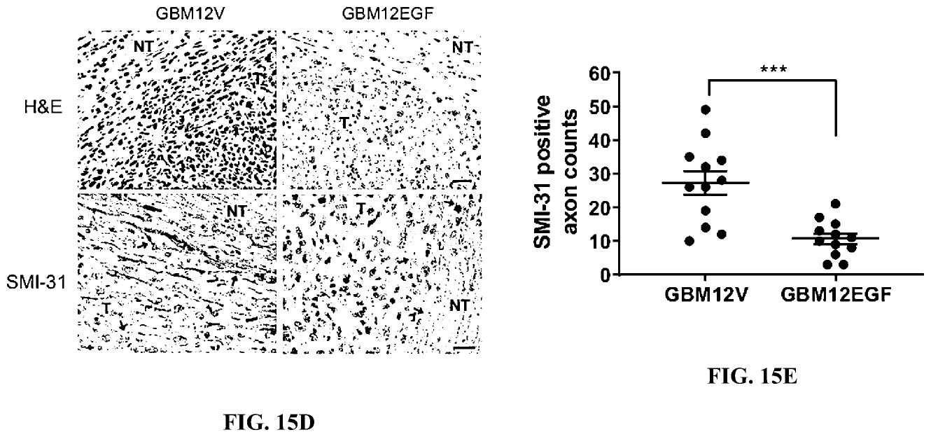

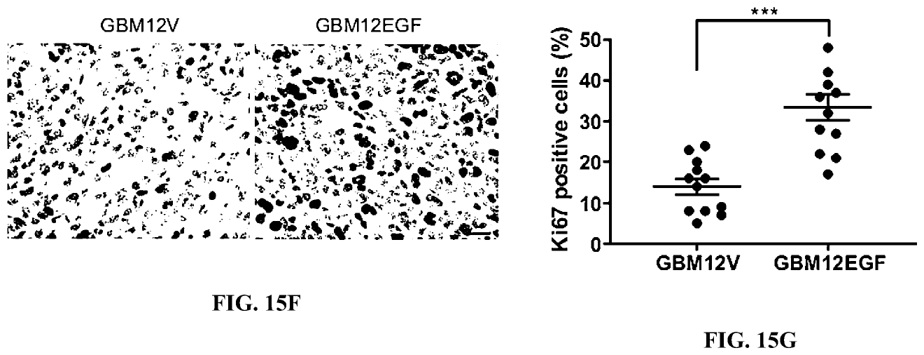

Finally, it was confirmed that stable expression of EGF in GBM12 also resulted in a similar phenotype to GBM12TGFα cells in an orthotopic mouse experiment. EGF overexpression was confirmed by ELISA, and phosphorylation of EGFR was also demonstrated in EGF overexpressing clones (FIG. 15A and FIG. 15B). This was followed by an animal experiment demonstrating that EGF overexpressing tumors are smaller, noninvasive and hyper proliferating and resulting in improved survival (FIG. 15C-G). Without wishing to be bound by theory, these data indicate that ligand activation of EGFR in multiple orthotopic PDX models results in tumors that are smaller, hyper-proliferating, non-invasive and with a better prognosis.

Referring to FIG. 15A-G, EGF overexpression prolongs survival, reduces invasiveness, and increases proliferation in an orthotopic glioblastoma mouse model. Specifically, FIG. 15A shows an ELISA for EGF in GBM12 stably transfected with EGF overexpressing (GBM12EGF_01, GBM12EGF_02) or empty vector (GBM12V). A Western blot analysis of pEGFR, EGFR, and BIN3 in EGF-overexpressing GBM12 clones is shown in FIG. 15B. The Western blot images are representative of three independent biological replicates. Actin served as loading control. FIG. 15C shows Kaplan-Meier survival curves of mice with orthotopic xenotransplant model of control GBM12V and GBM12EGF (GBM12EGF_02), n=6 each group. H&E staining and immunostaining of SMI-31 in GBM12V and GBM12EGF tumor tissue sections are shown in FIG. 15D. FIG. 15E shows quantification of SMI-31 counts in tumor tissue sections. Ki67 staining of GBM12V and GBM12EGF tumor tissue sections is shown in FIG. 15F. FIG. 15G shows quantification of Ki67 positive cells in tumor tissue sections. Scale bar: 25 μM. Data are represented as mean±SEM from three independent experiments. ***P<0.001, ****P<0.0001, unpaired two-tailed t-test.

6. BIN3 is Required for Ligand-Induced EGFR Effects on GBM Invasion In Vivo

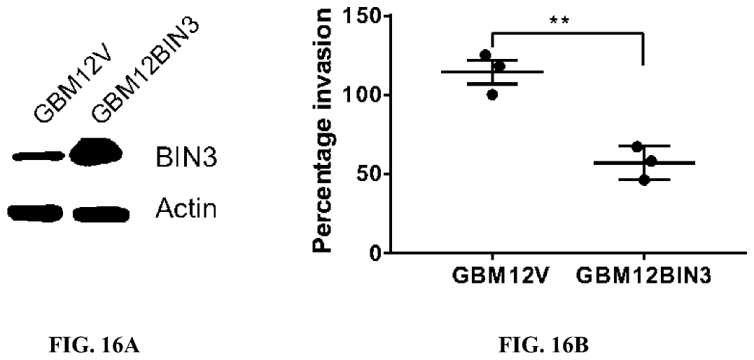

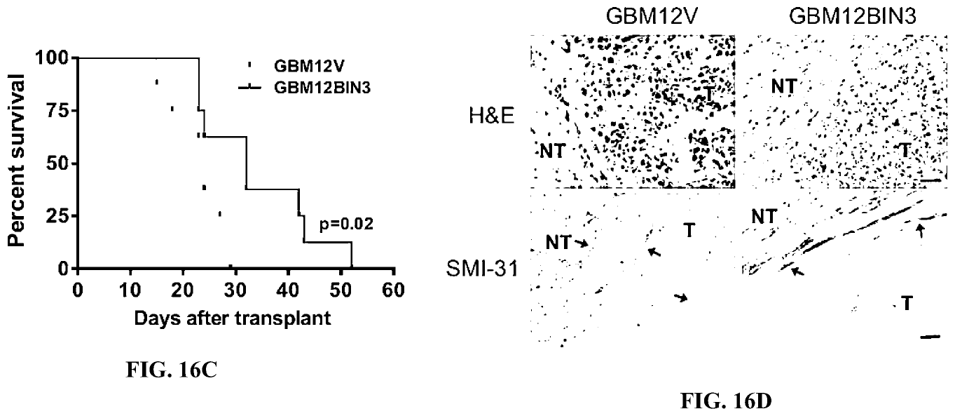

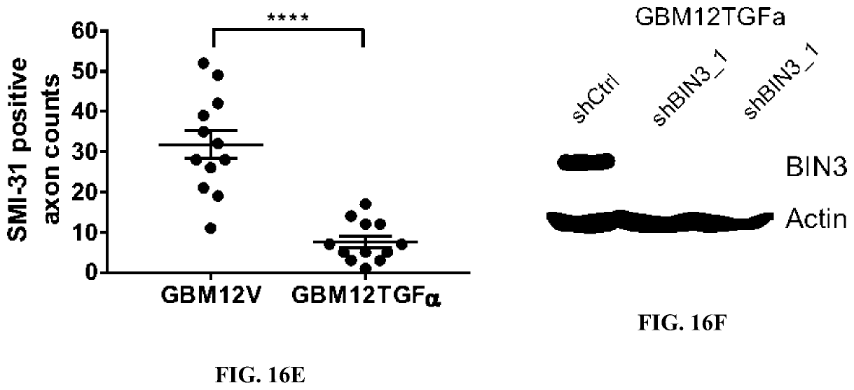

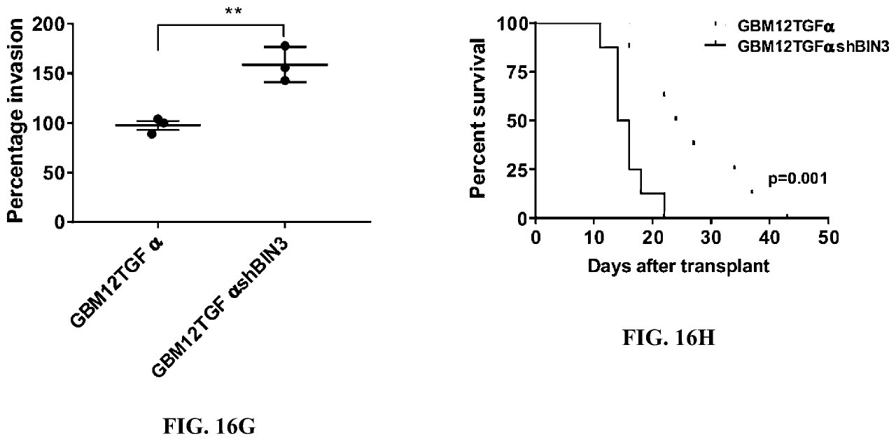

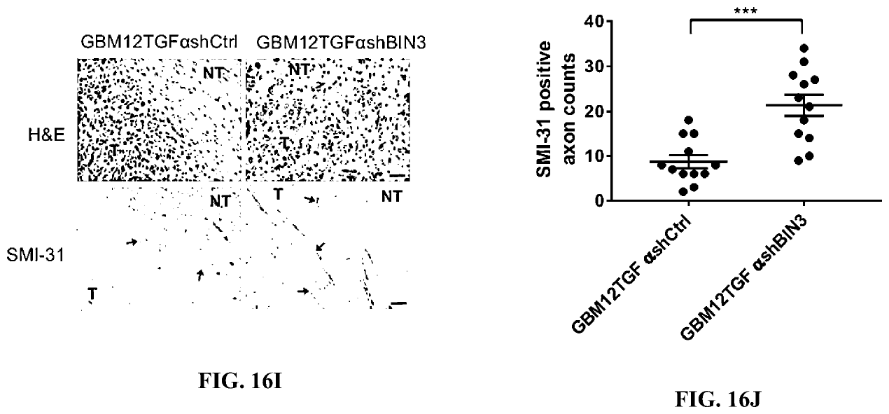

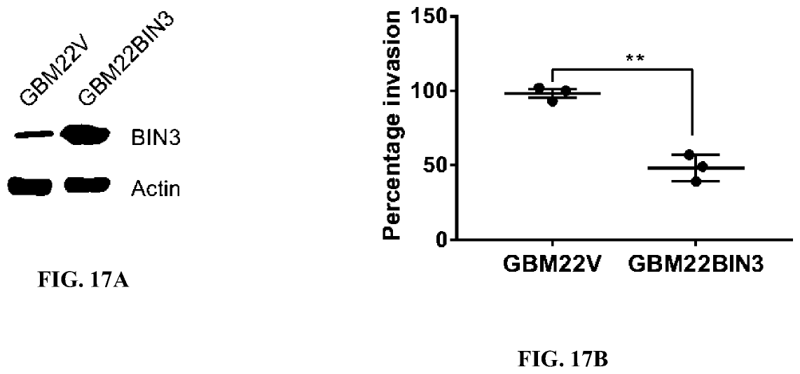

To examine the effect of BIN3 in a mouse model, BIN3 was stably overexpressed in GBM12 explant cultures (FIG. 16A and FIG. 17A). It was found that increased expression of BIN3 results in decreased invasiveness in transwell invasion assays (FIG. 16B and FIG. 17B). Next, a mouse orthotopic experiment was undertaken to examine the effect of BIN3 in an orthotopic model. BIN3 overexpression resulted in a better prognosis and resulted in non-invasive tumors (FIG. 16C-E). The requirement for BIN3 in EGFR-mediated suppression of invasion was subsequently examined in vivo. BIN3 was stably silenced in GBM12TGFα cells. This resulted in increased invasiveness in transwell invasion assays (FIG. 16F and FIG. 16G). BIN3 silencing results in a reversal of the effects of TGFα expression in GBM12 tumors in vivo. Thus, BIN3 silencing in GBM12TGFα cells results in formation of invasive tumors with a worse prognosis (FIG. 16H-J), confirming the critical role of BIN3 in regulating GBM invasion and prognosis.

Referring to FIG. 16A-J, BIN3 overexpression inhibits invasiveness. Western blot analysis of BIN3 in GBM12 stably overexpressing BIN3 (GBM12BIN3) or empty vector (GBM12V) is shown in FIG. 16A. FIG. 16B shows the results of a Matrigel gel invasion assay of GBM12V and GBM12BIN3. Kaplan-Meier survival curves of mice with orthotopic xenotransplant model of GBM12V and GBM12BIN3, n=8 per group, are shown in FIG. 16C. Representative H&E staining and immunostaining images for SMI-31 in GBM12V and GBM12BIN3 tumor tissue sections are shown in FIG. 16D. FIG. 16E shows the quantification of SMI-31 counts in GBM12V and GBM12BIN3 tumor tissue. Western blot analysis of BIN3 in GBM12TGFα stably transfected with control or BIN3 shRNA is shown in FIG. 16F. FIG. 16G shows the results of a Matrigel gel invasion assay of GBM12 TGFαshCtrl and GBM12TGFαshBIN3. Kaplan-Meier survival curves of orthotopic mouse xenotransplant model of GBM12TGFαshCtrl and GBM12TGFαshBIN3 (n=8 each group) are shown in FIG. 16H. FIG. 16I shows representative H&E staining and immunostaining for SMI-31 in GBM12TGFαshCtrl and GBM12TGFαshBIN3 tumor tissue sections. Quantification of SMI-31 counts in tumor tissues is shown in FIG. 16J. The Western blot images are representative of three independent biological replicates. Actin served as loading control. Scale bars: 25 μM. Data are represented as mean±SEM from three independent experiments. ** P<0.01, *** P<0.001, **** P<0.0001, n.s. not significant, unpaired two-tailed t-test.

Referring to FIG. 16A and FIG. 16B, BIN3 overexpression inhibits invasion of GBM22. Western blot analysis of BIN3 in GBM12 stably transfected with empty (GBM22V) or BIN3 expressing vector (GBM22BIN3) is shown in FIG. 16A. Matrigel invasion of GBM22V and GBM22BIN3 is shown in FIG. 16B. The Western blot images are representative of three independent biological replicates. Actin served as loading control. Data are represented as mean±SEM from three independent experiments. **P<0.01, unpaired two-tailed t-test.

7. Tofacitinib Upregulates BIN3 Levels and Decreases GBM Invasion

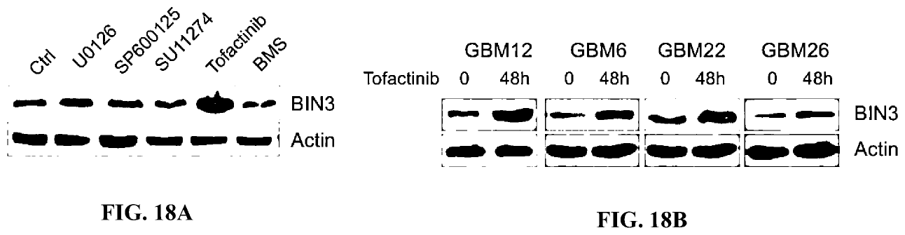

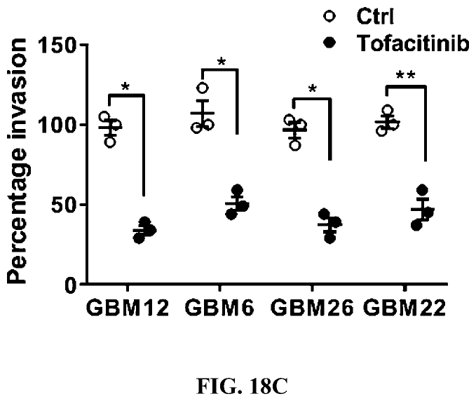

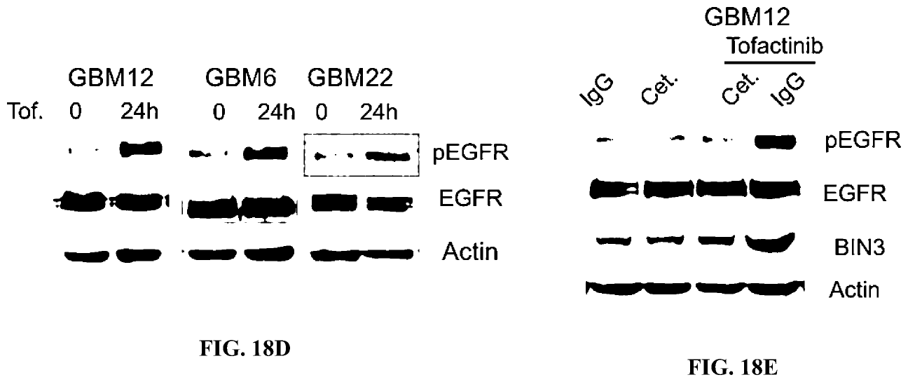

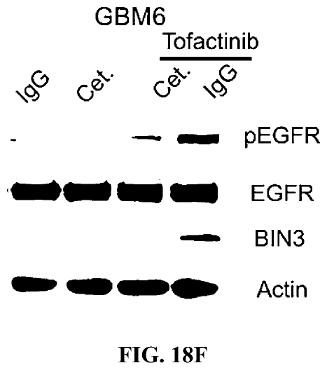

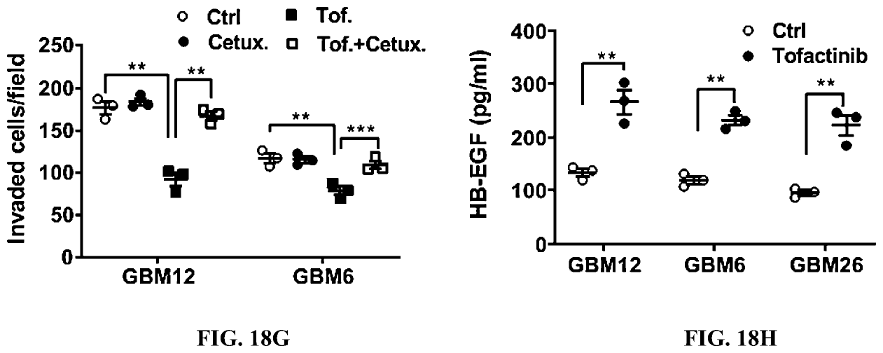

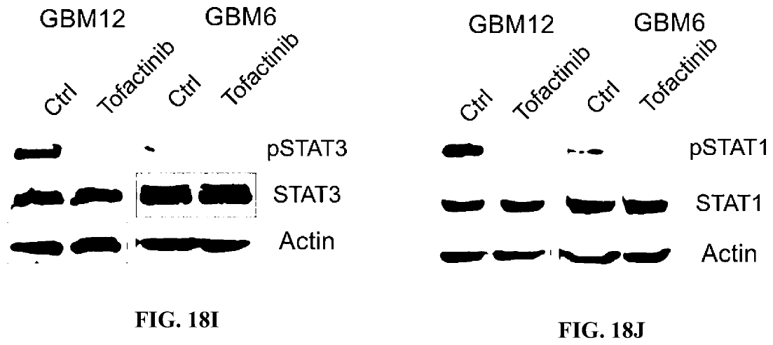

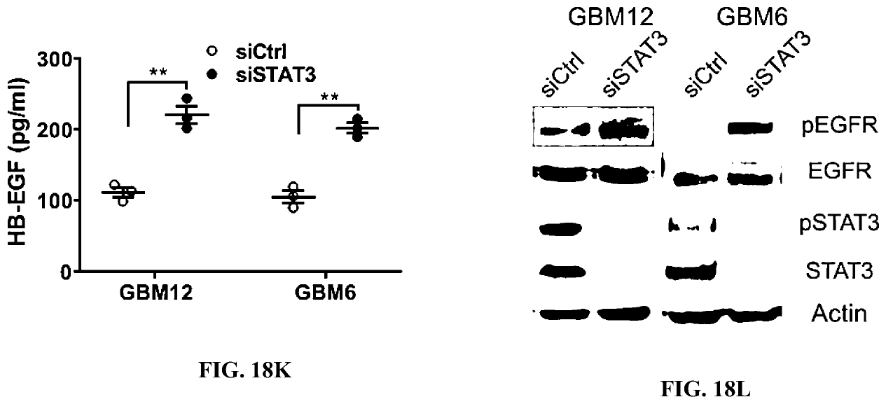

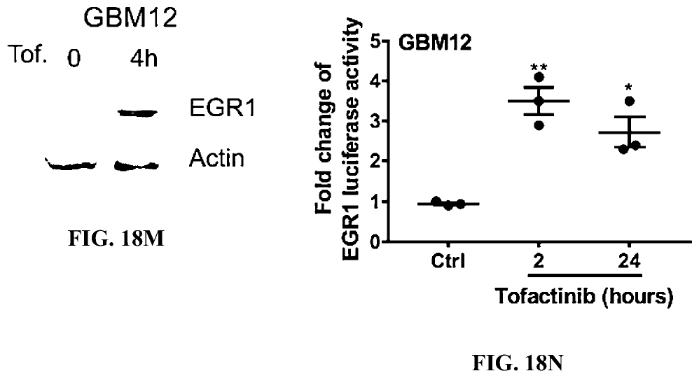

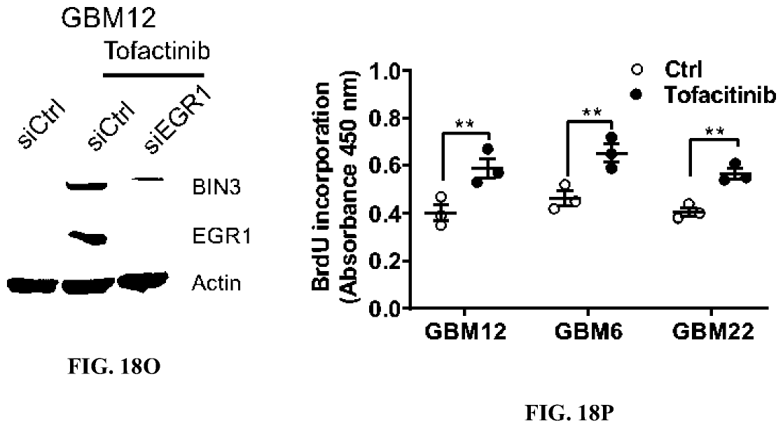

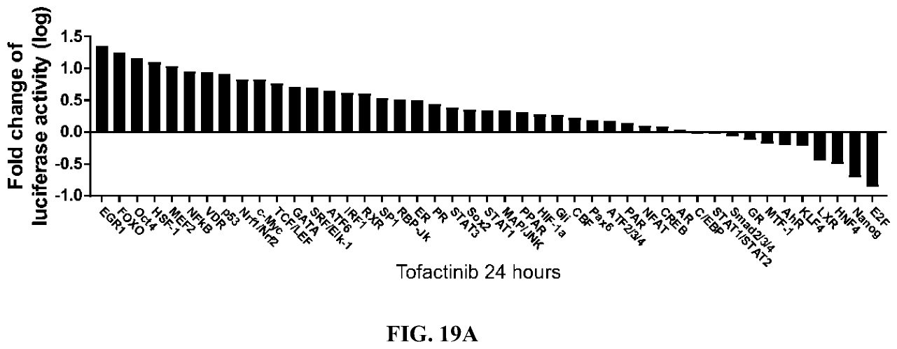

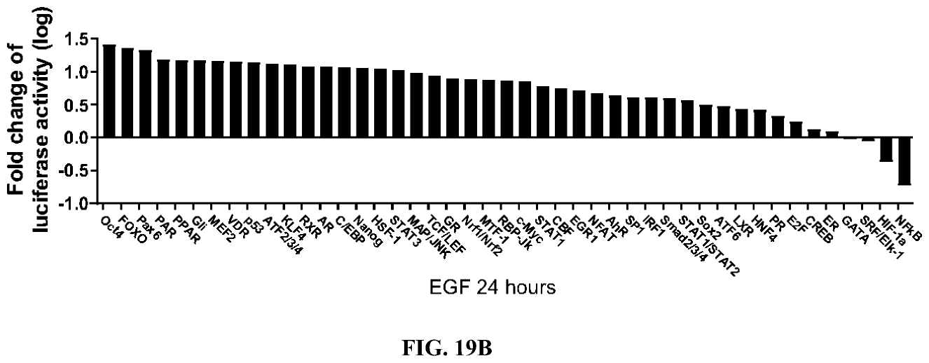

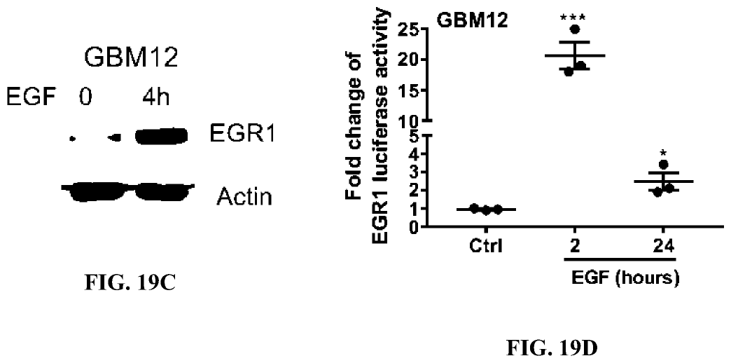

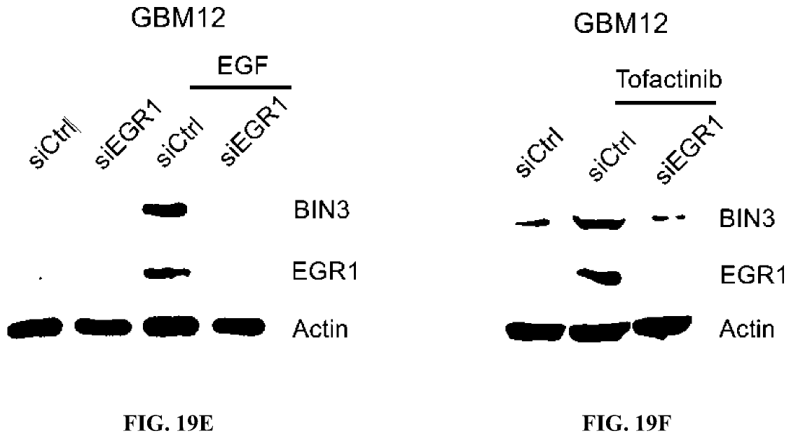

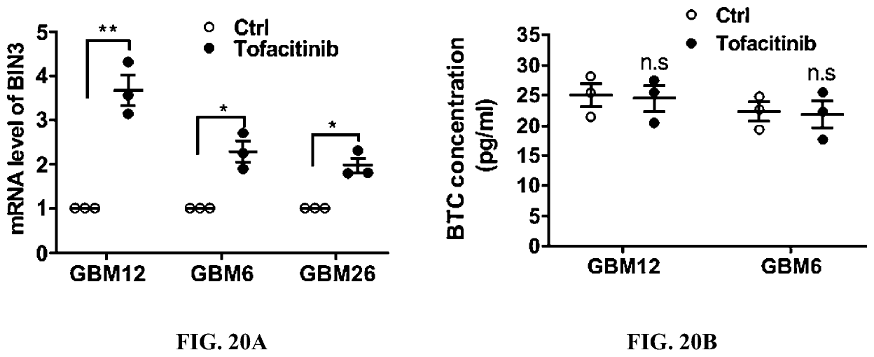

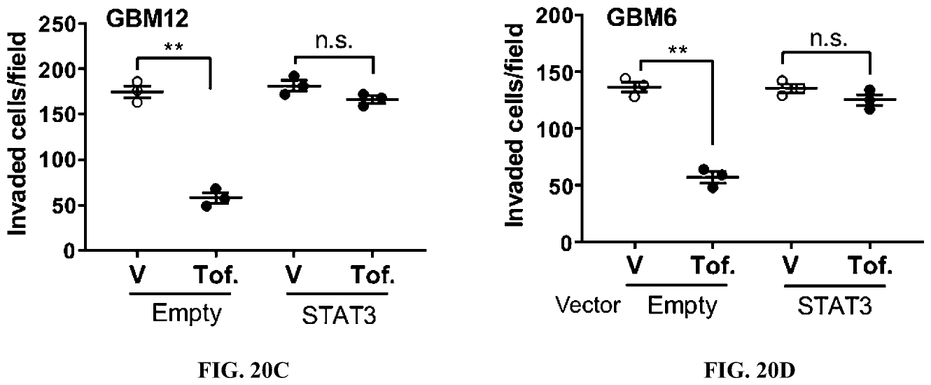

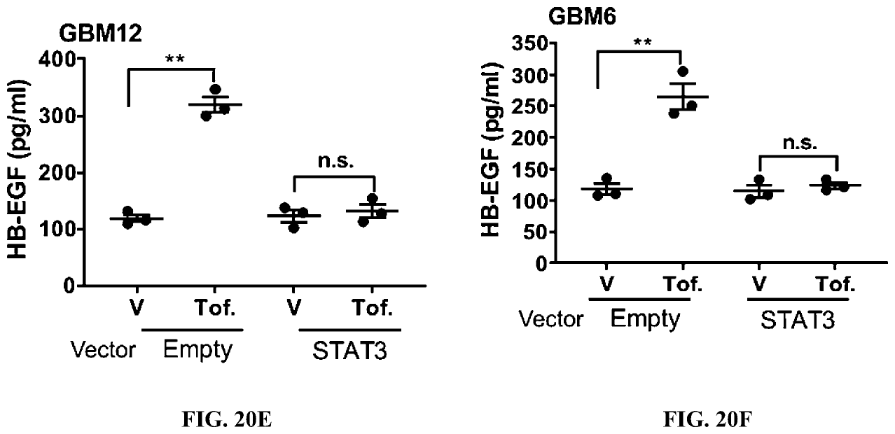

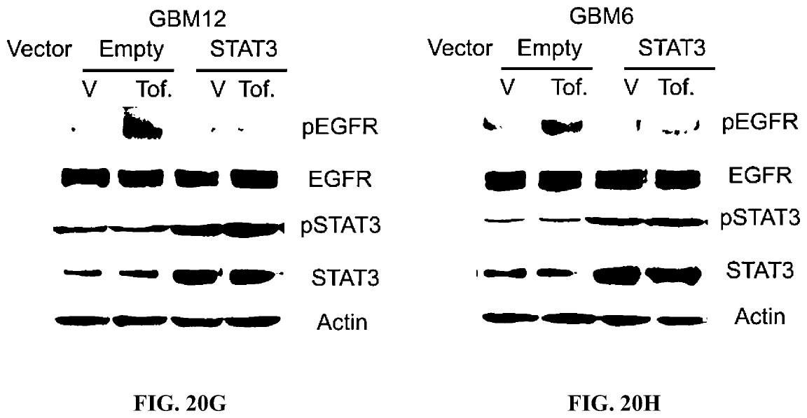

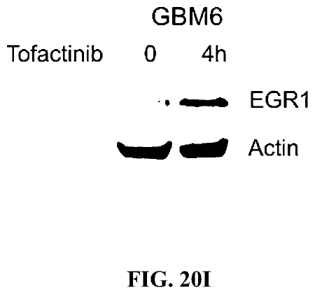

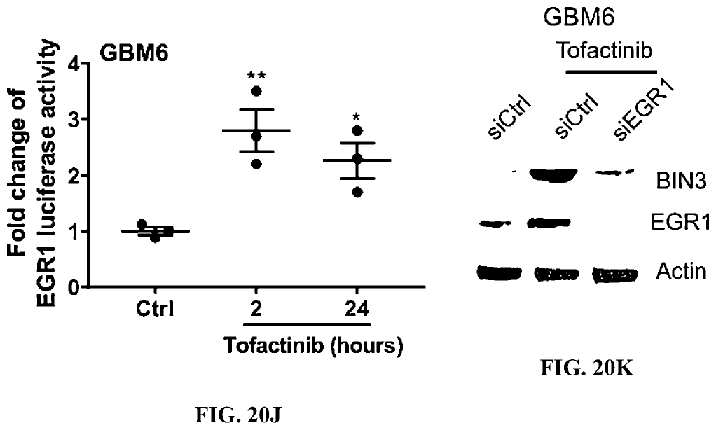

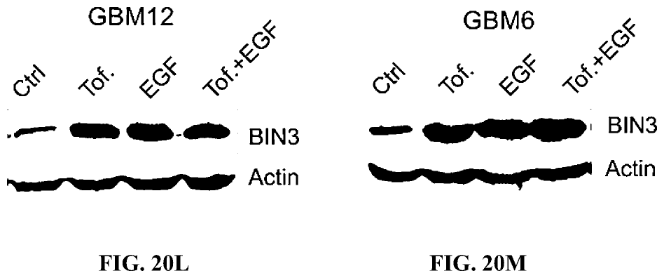

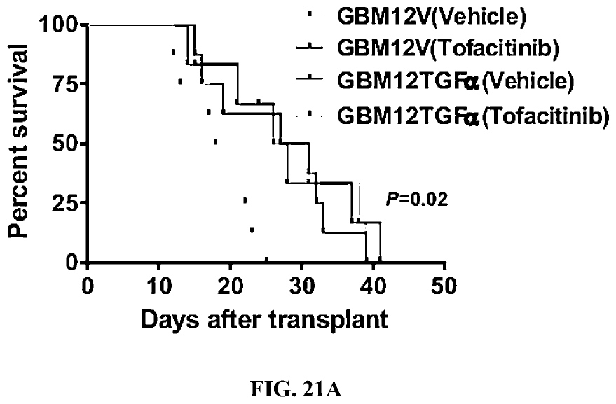

A drug that upregulates BIN3 and specifically inhibits invasion would be enormously useful. A panel of drugs that target key components of receptor tyrosine kinase signaling pathways was examined, and it was found that the Jak1/Jak3 inhibitor tofacitinib strongly upregulated BIN3 levels in multiple PDX lines (FIG. 18A and FIG. 18B). Consistent with this finding, tofacitinib inhibited invasiveness in multiple PDX lines in invasion assays (FIG. 18C). Similar to ligand-induced EGFR activity, tofacitinib also upregulates BIN3 at the mRNA level (FIG. 20A). Previous studies have indicated that inhibition of key downstream components of RTK signaling pathways leads to activation of RTKs such as the EGFR (Prahallad, et al. (2012) Unresponsiveness of colon cancer to BRAF(V600E) inhibition through feedback activation of EGFR, Nature 483: 100-103; Chandarlapaty, S., Sawai, A., Scaltriti, M., Rodrik-Outmezguine, V., Grbovic-Huezo, O., Serra, V., Majumder, P. K., Baselga, J., and Rosen, N. (2011) AKT inhibition relieves feedback suppression of receptor tyrosine kinase expression and activity, Cancer Cell 19: 58-71). The possibility that Jak inhibition by tofacitinib could result in EGFR activation was investigated. Indeed, it was found that tofacitinib results in a robust activation the EGFR (FIG. 18D). Furthermore, cetuximab which blocks ligand binding to the EGFR completely inhibits tofacitinib-induced EGFR activation, BIN3 upregulation, and suppression of invasion (FIG. 18E-G), indicating that tofacitinib upregulates BIN3 and inhibits invasion via a ligand-mediated activation of the EGFR. Consistent with this model, it was found that tofacitinib upregulates the EGFR ligand HB-EGF (FIG. 18H). Jak kinases signal by activating the STATs including STAT1 and STAT3. As expected, tofacitinib blocks phosphorylation of STAT1 and STAT3 (FIG. 18I and FIG. 18J). Next, it was found that siRNA knockdown of STAT3 mimicked the effect of tofacitinib and resulted in increased HB-EGF secretion and EGFR activation (FIG. 18K and FIG. 18L). This is consistent with a recent study reporting that STAT3 inhibition results in increased EGFR activation by increasing EGFR ligand, although the ligand identified in that study was betacellulin (Fan, et al. (2020) Betacellulin drives therapy resistance in glioblastoma, Neuro Oncol 22: 457-469). An upregulation of betacellulin was not found in response to tofacitinib (FIG. 20B) in this model. Thus, it is proposed that tofacitinib induces an HBEGF-induced activation of the EGFR, leading to increased BIN3 expression and decreased invasion. Jak inhibition by tofacitinib inhibits STAT3 activation. STAT3 normally represses HB-EGF transcription and STAT3 inhibition releases the inhibitory effect on HB-EGF. Thus, STAT3 overexpression rescues the effect of tofacitinib on invasion, EGFR activation and HB-EGF upregulation, (FIG. 20C-H). Furthermore, consistent with a model in which tofacitinib induces BIN3 via a ligand-mediated activation of the EGFR, it was shown that tofacitinib induces EGR1 levels and activity (FIG. 18M, FIG. 18N, FIG. 20I, and FIG. 20J). siRNA knockdown of EGR1 results in a loss of tofacitinib-induced BIN3 expression (FIG. 18O and FIG. 20K). Also, in cells treated with a saturating concentration of EGF, tofacitinib did not induce a further increase in BIN3 level (FIG. 20L-M). Finally, consistent with its effect on upregulation of the EGFR ligand HB-EGF, tofacitinib induced proliferation in glioma cells (FIG. 18P).

. . . Referring to FIG. 18A-P, tofacitinib inhibits invasion of PDXs by upregulation of BIN3. Western blot analysis of BIN3 in GBM12 treated with vehicle (Ctrl), U0126, SP600125, SU11274, tofacitinib, and BMS for 48 hours is shown in FIG. 18A. FIG. 18B shows Western blot analysis of BIN3 in PDXs treated with tofacitinib (1 μM) for 48 hours. Matrigel invasion of PDXs treated with 1 μM of tofacitinib for 24 hours is shown in FIG. 18C. FIG. 18D shows a Western blot analysis of pEGFR and EGFR in GBM12, GBM6, and GBM22 treated with tofacitinib (1 μM) for 24 hours. FIG. 18E shows Western blot analysis of pEGFR, EGFR, and BIN3 in GBM12 treated with tofacitinib in the absence or presence of cetuximab or IgG control for 24 hours. A similar experiment was performed in GBM6 (FIG. 18F). FIG. 18G shows the results of a Matrigel invasion experiment of GBM6 and GBM12 in the presence of tofacitinib or cetuximab or the combination. ELISA for HB-EGF in the supernatants of GBM12 and GBM6 treated with tofacitinib (1 μM) for 72 hours is shown in FIG. 18H. FIG. 18I shows a Western blot analysis of pSTAT3 and STAT3 in GBM12 and GBM6 treated with tofacitinib (1 μM) for 24 hours. FIG. 18J shows a Western blot analysis of pSTAT1 and STAT1 in GBM12 and GBM6 treated with tofacitinib (1 μM) for 24 hours. ELISA for HB-EGF in the supernatant of GBM12 and GBM6 72 hours after siRNA-mediated knockdown of STAT3 is shown in FIG. 18K. FIG. 18L shows a Western blot analysis of pEGFR and pSTAT3 in STAT3 siRNA knockdown GBM12. FIG. 18M shows a Western blot analysis of pEGFR and pSTAT3 in STAT3 siRNA knockdown GBM12. FIG. 18M shows a Western blot analysis of EGR1 in GBM12 treated with tofacitinib for 4 hours. Results of a EGR1 promoter luciferase assay of GBM12 treated with tofacitinib for 2 and 24 hours is shown in FIG. 18N. FIG. 18O shows a Western blot analysis of BIN3 in EGR1 siRNA knockdown GBM12 in response to tofacitinib (1 μM). Results of a Brdu incorporation assay of GBM12, GBM6, and GBM22 are shown in FIG. 18P. Cells were treated with/without 1 μM for 48 hours before the assay. The Western blot images are representative of three independent biological replicates. Actin served as loading control. Data are represented as mean±SEM from three independent experiments. *P<0.05, **P<0.01, ***P<0.001, unpaired two-tailed t-test.

Referring to FIG. 19A, the results of a Cignal 45-Pathway Reporter Array of GBM12 treated with/without tofacitinib (1 μM) for 24 hours are shown. Referring to FIG. 19B, the results of a Cignal 45-Pathway Reporter Array of GBM12 treated with/without EGF (50 ng/ml) for 24 hours are shown. FIG. 19C shows a Western blot analysis of EGR1 in GBM12 treated with EGF for 4 hours is shown. The results of an EGR1 promoter luciferase assay of GBM12 treated with EGF is shown in FIG. 19D. FIG. 19E shows a Western blot analysis of BIN3 in EGR1 siRNA knockdown GBM12 in the absence and presence of EGF. FIG. 19F shows a Western blot analysis of BIN3 in EGR1 siRNA knockdown GBM12 in the absence and presence of EGF.

Referring to FIG. 20A-M, EGR1 is required for both EGF- and tofacitinib-induced BIN3 expression. BIN3 mRNA expression in PDXs treated with tofacitinib (1 μM) for 24 hours is shown in FIG. 20A. The results of an ELISA for BTC in the supernatants of GBM12 and GBM6 are shown in FIG. 20B. Supernatants were collected in cells treated with tofacitinib (1 μM) for 72 hours. FIG. 20C shows the results of a Matrigel invasion assay of STA3 overexpressing GBM12 in response to tofacitinib. Cells were transiently transfected with empty vector or STAT3 expression vector for 48 hours, transfected cells were seeded on Matrigel coated inserts and treated with vehicle (V) or tofacitinib (Tof 1 μM) for 24 hours. Similar experiments were performed in GBM6 (FIG. 20D). The results of an ELISA for HB-EGF in STAT3 overexpressing GBM12 treated with toactinib are shown in FIG. 20E. Cells were transiently transfected with empty or STAT3 expression vector for 48 hours, transfected cells were treated with vehicle or tofacitinib for 48 hours and supernatants were collected to measure HB-EGF expression using ELISA kit. Similar experiments were performed in GBM6 (FIG. 20F). FIG. 20G and FIG. 20H show Western blot analysis of pEGFR, EGFR, pSTAT3, STAT3 in STAT3 overexpressing GBM12 and GBM6 in response to tofacitinib. Cells were transiently transfected with empty or STAT3 expression vector and treated with tofacitinib (1 μM) for 24 hours. FIG. 20I shows Western blot analysis of EGR1 in GBM6 treated with EGF (50 ng/ml) for 24 hours. The results of an EGR1 promoter luciferase assay in GBM6 in response to EGF treatment are shown in FIG. 20J. FIG. 20K shows Western blot analysis of BIN3 in EGR1 siRNA knockdown GBM6 in response to tofacitinib (1 μM). FIG. 20L and FIG. 20M show Western blot analysis of BIN3 in GBM12 and GBM6 treated with tofacitinib, EGF, or the combination of both for 48 hours. The Western blot images are representative of three independent biological replicates. Actin served as loading control. Data are represented as mean±SEM from three independent experiments. * P<0.05, **P<0.01, ****P<0.0001, unpaired two-tailed t-test.

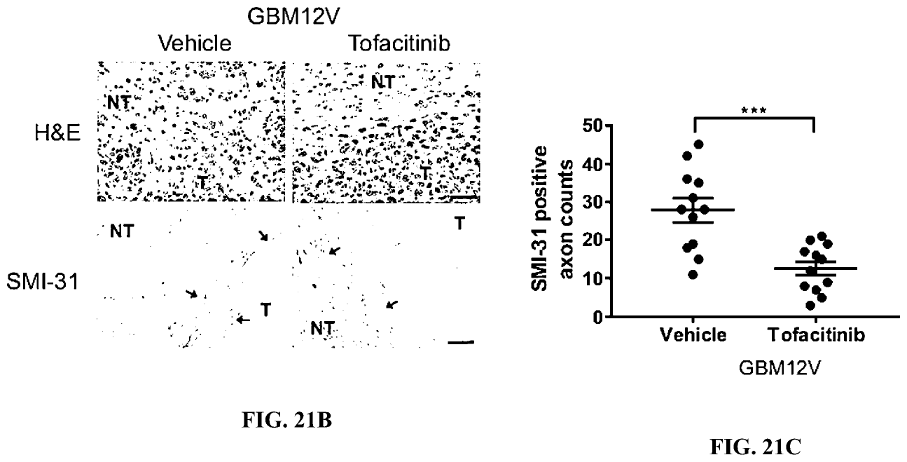

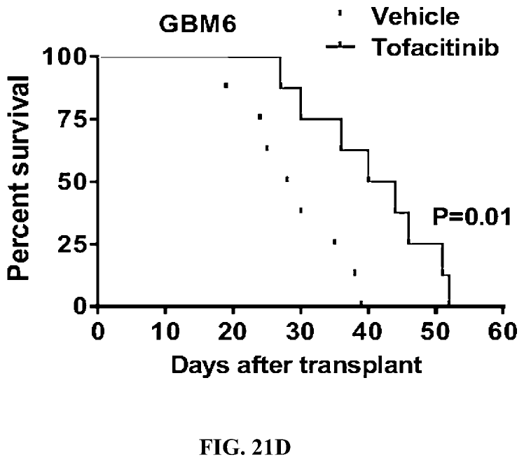

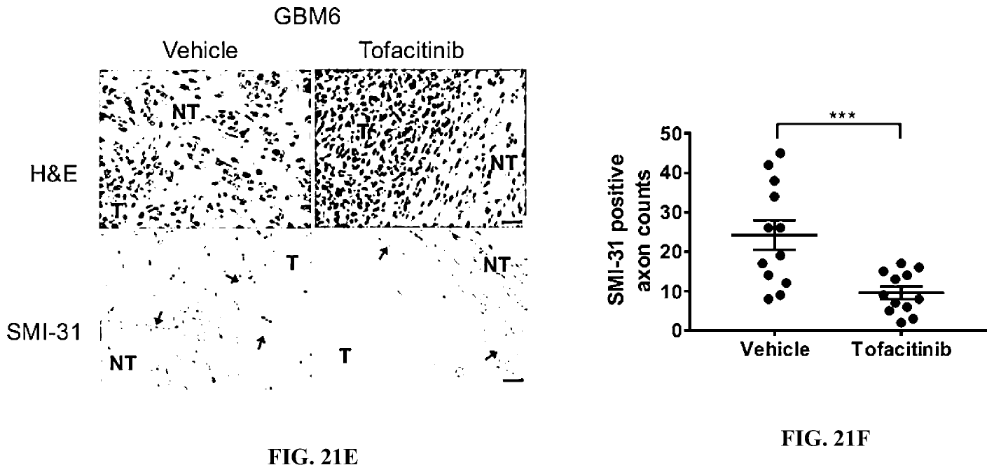

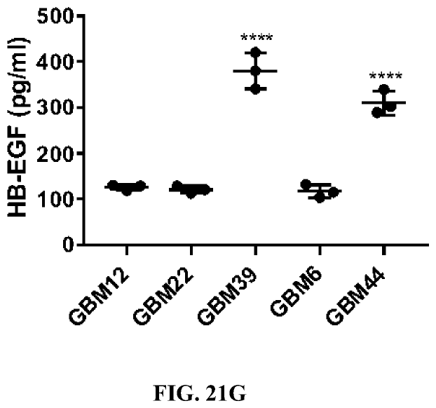

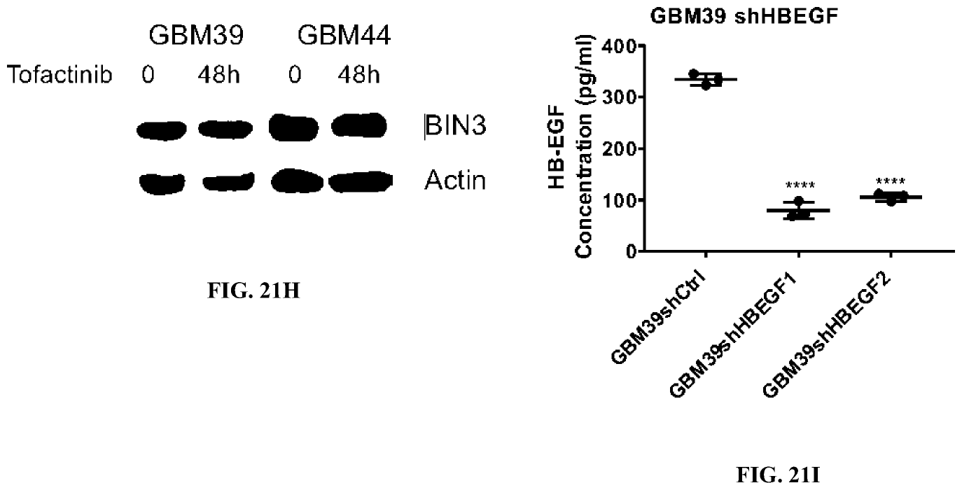

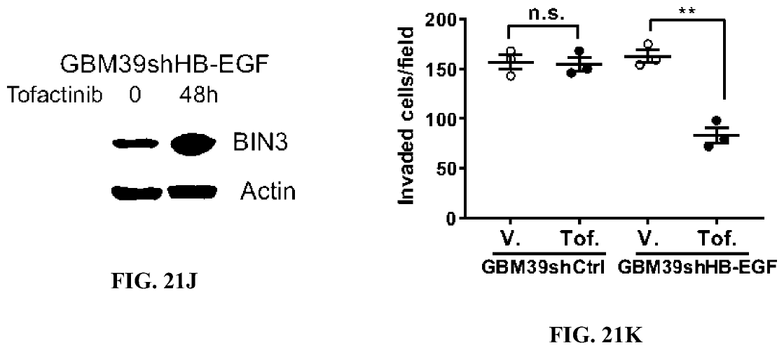

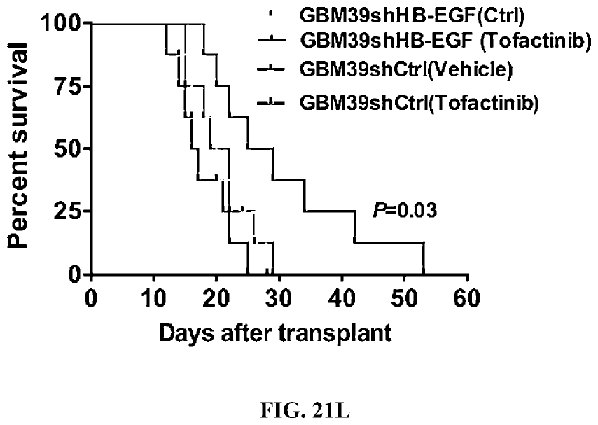

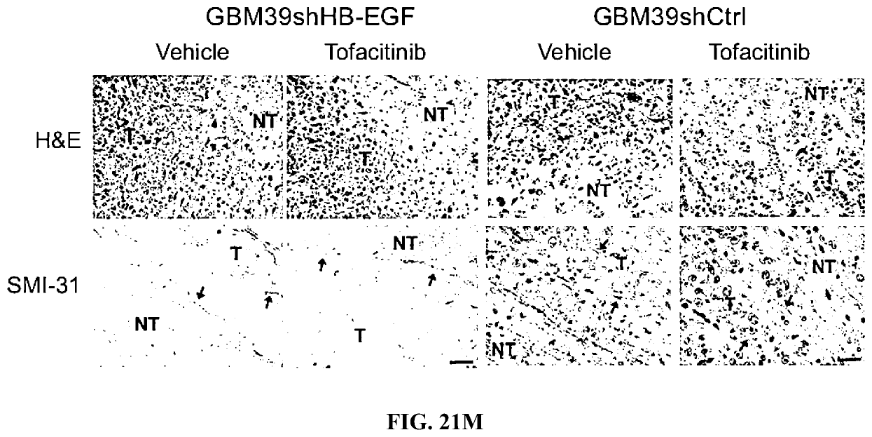

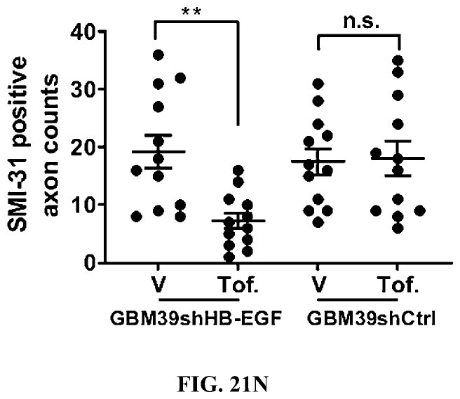

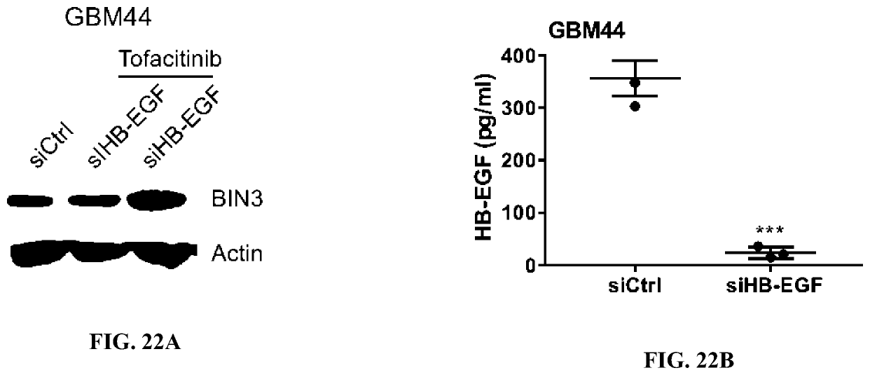

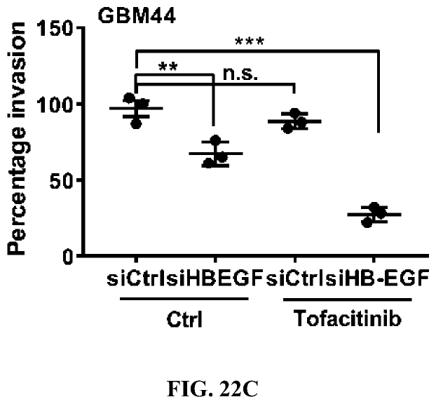

Next, whether tofacitinib is effective in a mouse model was examined. Based on ex vivo experiments suggesting that tofacitinib failed to upregulate BIN3 levels in EGF treated cells, it was predicted that tumors expressing a low level of EGFR ligand would be more responsive to tofacitinib. Thus, the effect of tofacitinib in mouse intracranial GBM tumors generated from GBM12TGFα cells or from GBM12V (vector transfected) cells were compared. Indeed, as noted previously, while GBM12TGFα tumors grow more slowly compared to vector transfected tumors, there is no additional benefit with tofacitinib. GBM12V tumors, on the other handed, responded to tofacitinib treatment with a significant improvement in survival and decreased invasion (FIG. 21A-C). Similar results were detected with the EGFRvIII expressing PDX line GBM6 (FIG. 21D-F). To confirm the observation that low ligand and upregulation of BIN3 renders tumors responsive to tofacitinib, two PDX tumors that have endogenous high EGFR ligand and high BIN3 levels were identified (FIG. 21G). It was found that these tumors do not respond to tofacitinib by upregulation of BIN3 (FIG. 21H). HBEGF was silenced in GBM39 cells, and it was found that tofacinib could now upregulate BIN3 in GBM39HBEGF cells (FIG. 21I and FIG. 21J). GBM39HBEGF cells are more responsive to tofacitinib compared to GBM39C cells in invasion assays (FIG. 21K). A similar result was found with the GBM44 PDX line (FIG. 22A-C). Next, whether silencing of HBEGF confers responsiveness to tofacitinib in vivo was examined, and it was found that silencing HBEGF also renders orthotopic GBM39HBEGF tumors were also more responsive to tofacitinib compared to controls (FIG. 21L-N). As expected, the survival of tofacitinib treated tumors is improved and the tumors are less invasive. Without wishing to be bound by theory, these data suggest that a tofacitinib induced increase in BIN3 level triggers the anti-invasion activity of BIN3.

Referring to FIG. 21A-N, tofacitinib prolongs survival of mice bearing orthotopic glioblastoma tumors. Kaplan-Meier survival curves of mice with orthotopic xenotransplant model of GBM12V and GBM12TGFα treated with vehicle or tofacitinib (50 mg/kg) (n=8 in GBM12 TGFα groups, n=6 in GBM12V groups) are shown in FIG. 21A. P=0.02 Vehicle vs. tofacitinib in GBM12TGFα groups. Representative H&E staining and immunostaining of SMI-31 showing invasiveness in GBM12 orthotropic tumor from vehicle and tofacitinib treated mice is illustrated in FIG. 21B. FIG. 21C shows the quantification of SMI-31 counts in tumor tissue. Kaplan-Meier survival curves of mice with orthotopic xenotransplant model of GBM6 treated with vehicle or tofacitinib (50 mg/kg) are shown in FIG. 21D. Representative H&E staining and immunostaining of SMI-31 showing invasiveness in GBM6 orthotropic tumor from vehicle and tofacitinib treated mice is illustrated in FIG. 21E. FIG. 21F shows the quantification of SMI-31 counts in tumor tissue. ELISA for HB-EGF in the supernatant of PDXs is shown in FIG. 21G. FIG. 21H shows Western blot analysis of BIN3 in GBM39 and GBM44 treated with tofacitinib (1 μM) for 48 hours. Knockdown efficiency of HB-EGF in GBM39 control shRNA (GBM39shCtrl) and HB-EGF shRNA (GBM39shHB-EGF_1, GBM39shHB-EGF_2) clones was confirmed by ELISA (FIG. 21I). FIG. 21J shows Western blot analysis of BIN3 in GBM39shHB-EGF treated with tofacitinib for 48 hours. FIG. 21K shows the results of a Matrigel invasion assay of the GBM39shCtrl and GBM39shHB-EGF (GBM39shHB-EGF_1) in response to tofacitinib (1 μM). Kaplan-Meier survival curves of mice with orthotopic xenotransplant model of GBM39shCtrl and GBM39shHB-EGF treated with vehicle or tofacitinib (n=8 each group) are shown in FIG. 21L. Representative H&E staining and immunostaining for SMI-31 showing invasiveness in GBMshHB-EGF and GBM39shCtrl orthotopic tumor from vehicle and tofacitinib treated mice are shown in FIG. 21M. FIG. 21N shows representative quantification of SMI-31 counts in tumor tissue. The Western blot images are representative of three independent biological replicates. Actin served as loading control. Scale bars: 25 μM. Data are represented as mean±SEM from three independent experiments. ***P<0.001, ***P<0.0001, n.s. not significant, unpaired two-tailed t-test.

Referring to FIG. 22A-C, tofacitinib inhibits invasion of GBM44 with HB-EGF knockdown. A Western blot showing BIN3 expression in GBM44 transfected with scrambled (siCtrl) or HB-EGF siRNA in the presence or absence of 1 μM of tofacitinib is shown in FIG. 22A. HB-EGF siRNA knockdown efficiency of GBM44 was confirmed by ELISA (FIG. 22B). FIG. 22C shows the results of a Matrigel invasion assay of GBM44 transfected with scrambled or HB-EGF siRNA in the presence or absence of tofacitinib (1 μM). Data are represented as mean±SEM from three independent experiments. ** P<0.01, *** P<0.001, n.s. not significant, unpaired two-tailed t-test.

8. Constitutive EGFR Signaling Drives Invasion by Upregulating EMP1

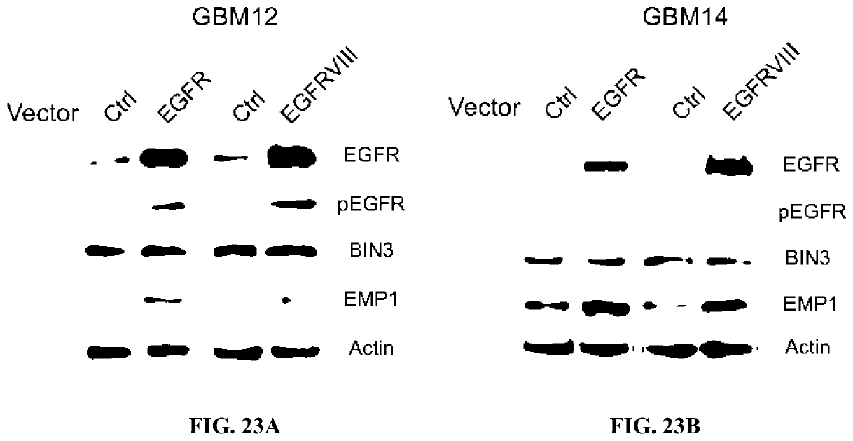

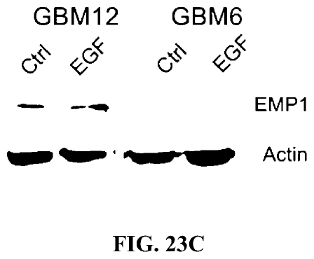

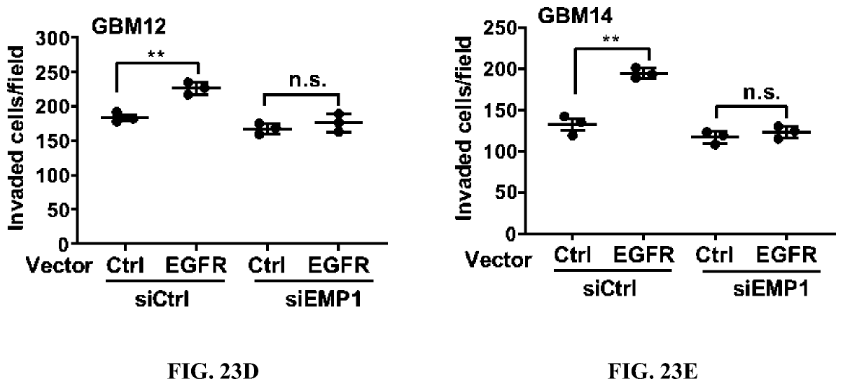

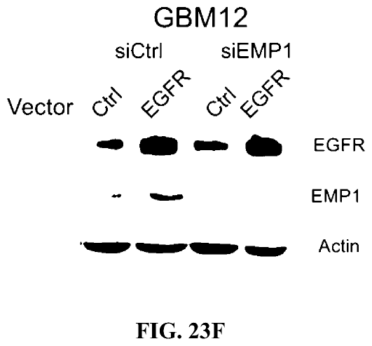

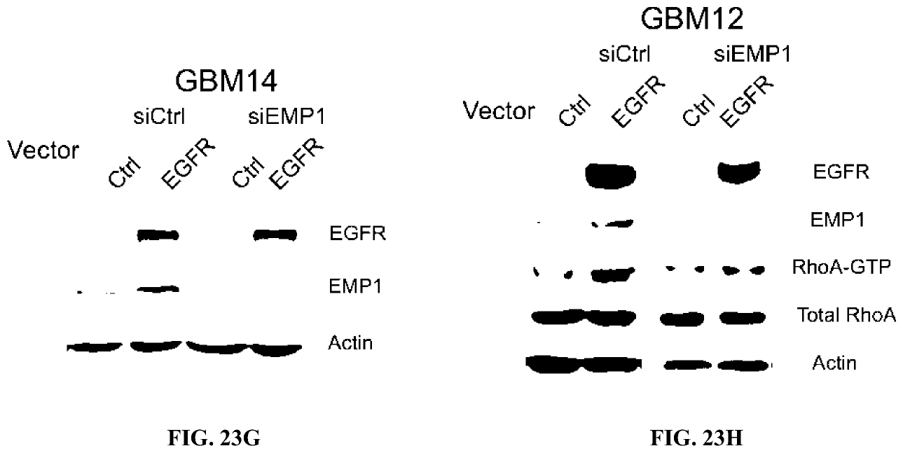

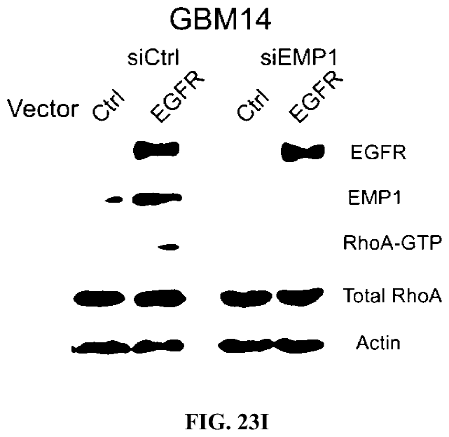

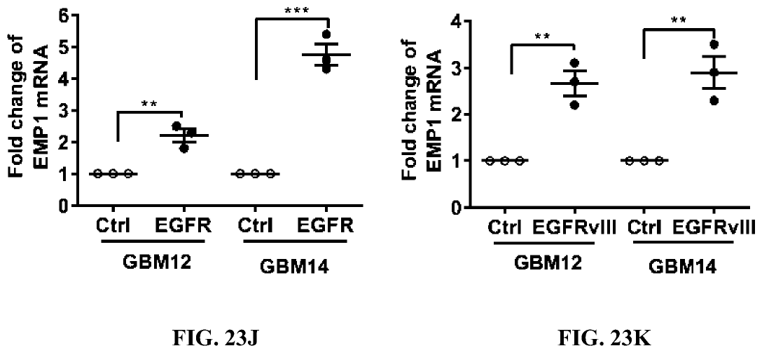

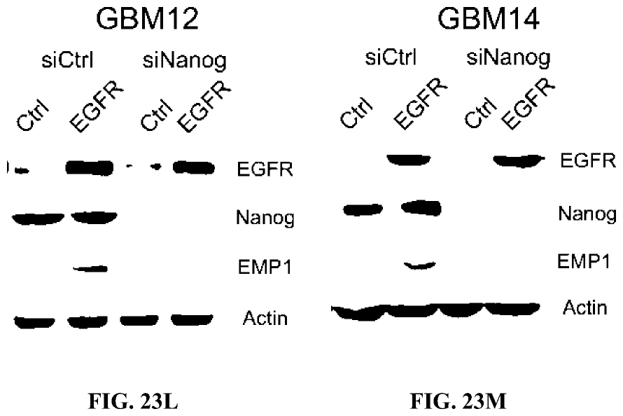

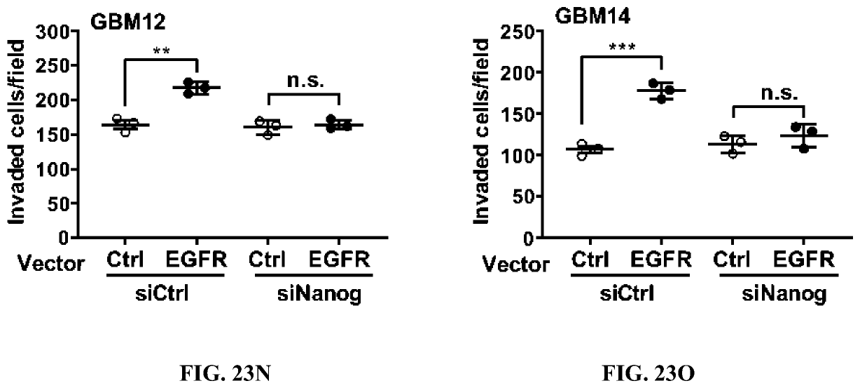

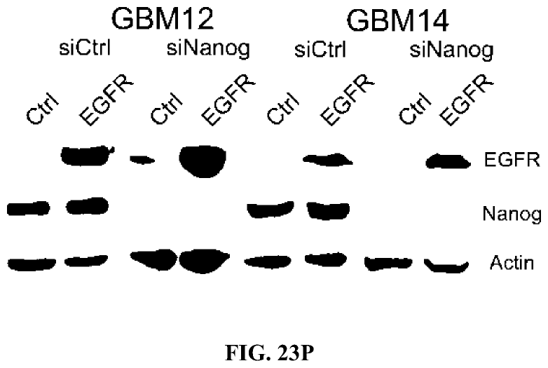

Studies indicate that ligand-mediated EGFR activation inhibits invasion by upregulation of BIN3. It was found that constitutive EGFR signaling does not significantly affect BIN3 levels (FIG. 23A and FIG. 23B). To understand the mechanism of constitutive EGFR signaling mediated invasion, the previous microarray analysis was reviewed to identify genes upregulated only by constitutive EGFR signaling (Ramnarain, et al. (2006) Differential gene expression analysis reveals generation of an autocrine loop by a mutant epidermal growth factor receptor in glioma cells, Cancer Res 66: 867-874). Epithelial membrane protein 1 (EMP1) was identified as a gene upregulated by constitutive EGFR signaling. Although in a previous study EMP1 was identified as a gene upregulated by EGFRvIII, it was found that EMP1 can be upregulated by constitutive EGFRwt or EGFRvIII expression (FIG. 23A and FIG. 23B). Addition of EGFR ligand does not affect EMP1 level (FIG. 23C). EMP1 was prioritized for further study because it has been reported to play a key role in cancer invasiveness and acts by increasing activity of RhoGTPases (Ahmat Amin, et al. (2019) The Pivotal Roles of the Epithelial Membrane Protein Family in Cancer Invasiveness and Metastasis, Cancers 11). It was found that EMP1 is essential for constitutive EGFR signaling driven invasiveness, since siRNA mediated knockdown of EMP1 resulted in a loss of invasiveness resulting from EGFR expression in GBM lines (FIG. 23D-G). It was confirmed that constitutive EGFR signaling resulted in RhoA activation and that siRNA knockdown of EMP1 resulted in a loss of EGFR mediated Rho activation (FIG. 23H and FIG. 23I).

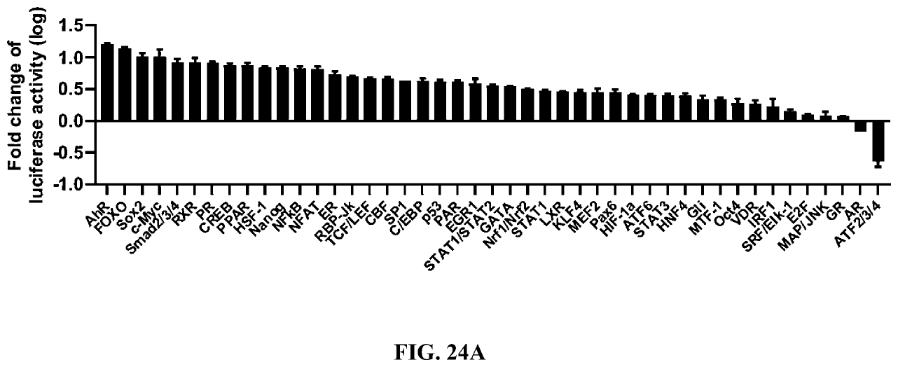

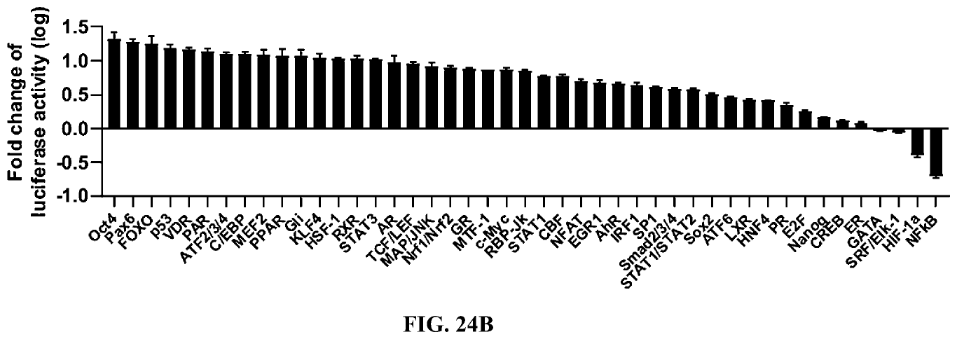

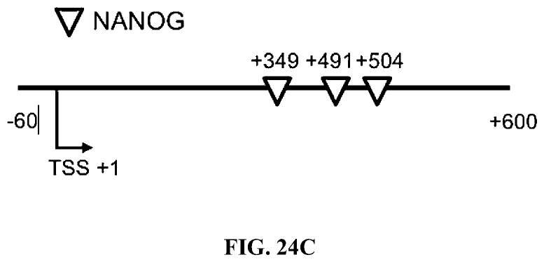

It was confirmed that EMP1 is upregulated at the mRNA level by constitutive EGFRwt or EGFRvIII signaling (FIG. 23J and FIG. 23K). Next, which transcription factors were upregulated by constitutive EGFR signaling was investigated by undertaking the CIGNAL reporter assay that assesses activity of 45 transcription factors (FIG. 24A and FIG. 24B). Nanog was identified as a putative candidate because its activity was upregulated by constitutive but not by ligand-activated EGFR signaling and Nanog binding sites were identified in the EMP1 promoter (FIG. 24C). Furthermore, siRNA knockdown of Nanog blocks EGFR mediated upregulation of EMP1 and also blocks EGFR mediated increase in invasiveness (FIG. 23L-P). Thus, a Nanog-EMP1 signaling axis drives constitutive EGFR mediated increased invasion.

Referring to FIG. 23A-P, EGFR induced EMP1 overexpression drives invasion and is mediated by Nanog. Specifically, cells were transiently transfected with empty or EGFRwt or EGFRvIII vector for 48 hours, and subjected to Western blot analysis for EGFR, pEGFR, BIN3 and EMP1 (FIG. 23A). Similar experiments were performed in GBM14 (FIG. 23B). FIG. 23C shows Western blot analysis of EMP1 in GBM12 and GBM6 treated with EGF (50 ng/ml) for 24 hours. FIG. 23D shows the results of Matrigel invasion of EMP1 siRNA knockdown GBM12 cells transfected with empty or EGFR vectors. Similar experiments were performed in GBM14 (FIG. 23E). Efficiency of EMP1 knockdown and EGFR overexpression was confirmed by Western blot analysis (FIG. 23F and FIG. 23G). Cells were transfected with control or EMP1 siRNA for 48 hours, followed by transfection with empty or EGFR expression vectors for 48 hours, and subjected to Western blot analysis of RhoA-GTP, Total RhoA, EGFR, and EMP1 (FIG. 23H). Similar experiments were performed in GBM14 (FIG. 23I). FIG. 23J and FIG. 23K show EMP1 mRNA levels in EGFR and EGFRvIII overexpressing GBM12 and GBM14, measured by RT-PCR. EMP1 protein levels induced by EGFR or EGFRvIII overexpression were described in FIG. 23A and FIG. 23B. Cells were transfected with control or Nanog siRNA for 48 hours, followed by transfection with empty or EGFR vectors for additional 48 hours, and subjected to Western blot analysis of EMP1, Nanog, EGFR, and Actin (FIG. 23L). Similar experiments were performed in GBM14 (FIG. 23M). FIG. 23N shows the results of Matrigel invasion of Nanog siRNA knockdown GBM12 transfected with empty or EGFR vectors, invasion assay was performed after 48 hours. Similar experiments were performed in GBM14 (FIG. 23O). Efficiency of Nanog siRNA knockdown and EGFR overexpression was confirmed by Western blot analysis is shown in FIG. 23P. The Western blot images are representative of three independent biological replicates. Actin served as loading control. Data are represented as mean±SEM from three independent experiments. ***P<0.001, ***P<0.0001, n.s. not significant, unpaired two-tailed t-test.

Referring to FIG. 24A-C, results of a Cignal 45 pathway reporter assay and schematic diagram of EMP-1 promoter are shown. Specifically, FIG. 24A shows the results of a Cignal 45-Pathway Reporter Array of EGFR overexpressing GBM12. Cells were transiently transfected with empty or EGFRwt vector for 48 hours before assessing transcription factor activation using the Cignal 45 assay. Fold change (log 2) was calculated based on normalized luciferase activity of the EGFR overexpressing cells relative to the control cells. FIG. 24B shows the results of a Cignal 45-Pathway Reporter Array of GBM12 treated with or without EGF (50 ng/ml) for 24 hours. Fold change (log 2) was calculated based on normalized luciferase activity of the EGF treated cells relative to the untreated. Data represent mean±SEM of duplicate wells. A schematic diagram of the putative NANOG binding sites in EMP1 promoter region is shown in FIG. 24C.

9. Single Cell Analysis Reveals that Ligand-Mediated Activation of EGFR Suppresses Invasion

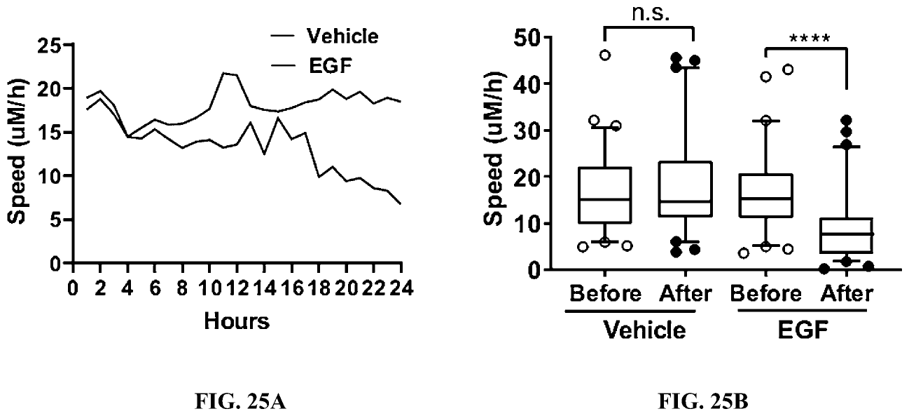

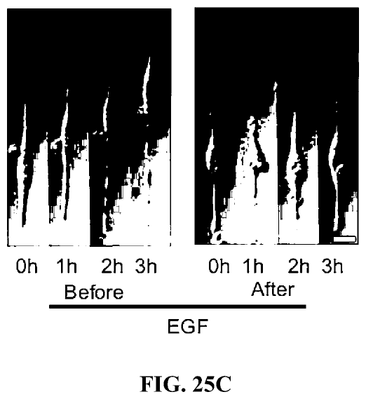

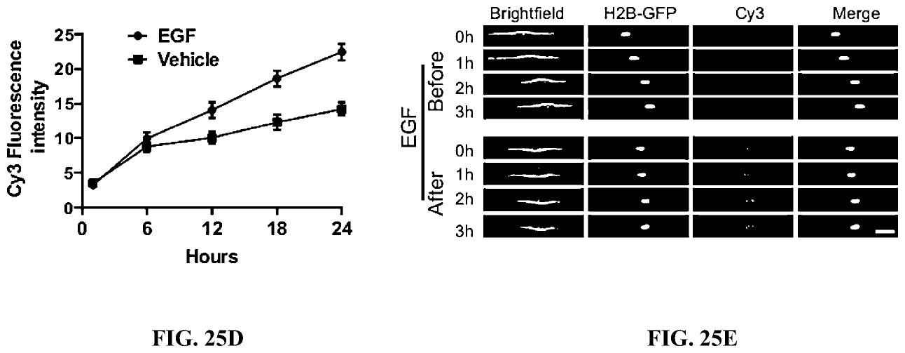

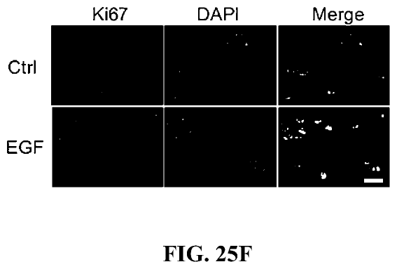

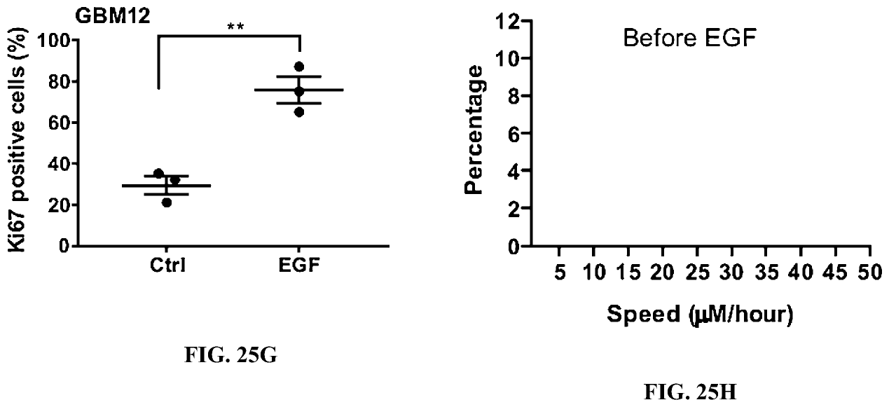

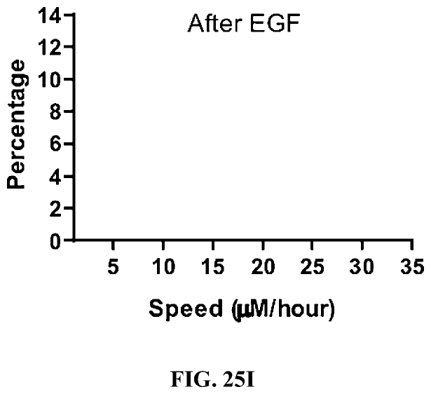

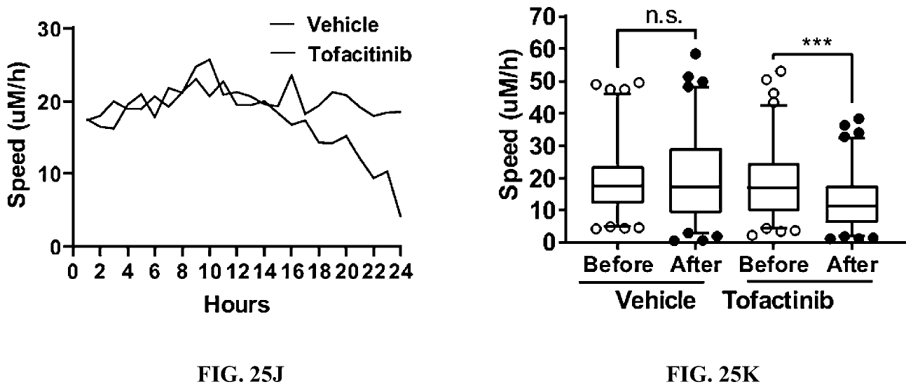

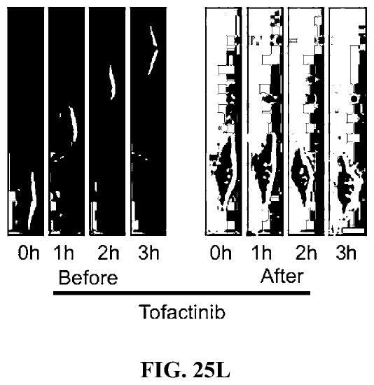

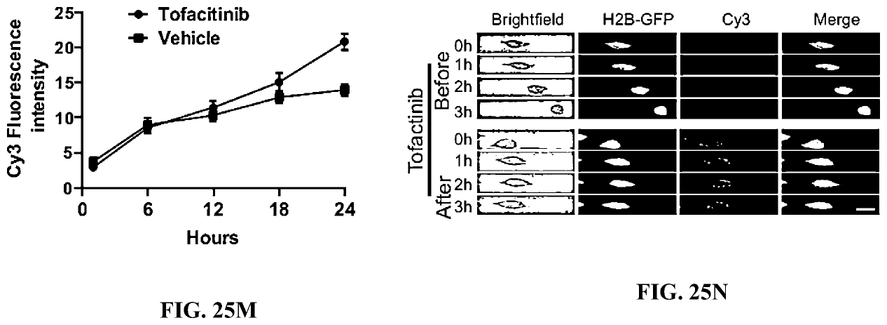

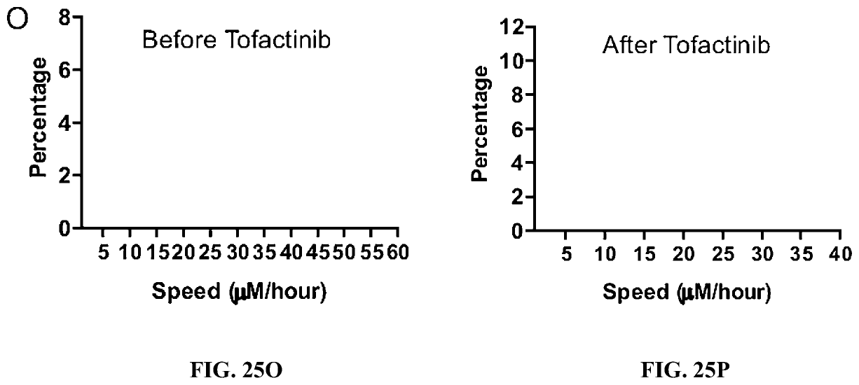

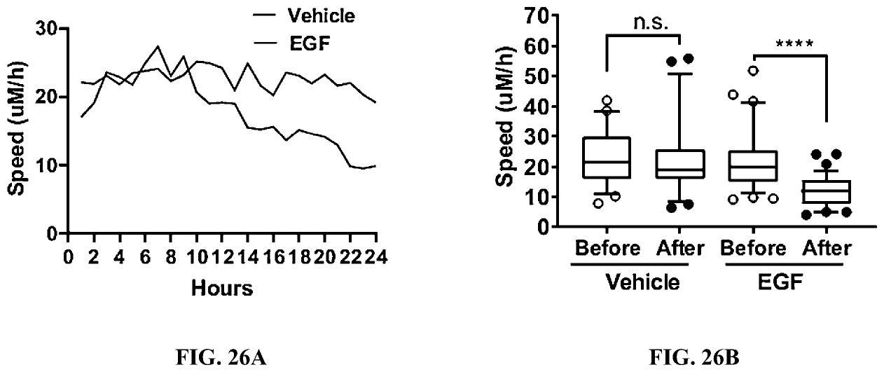

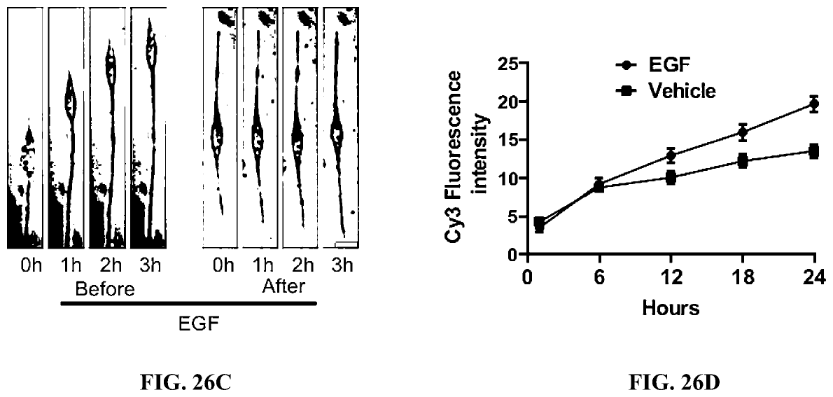

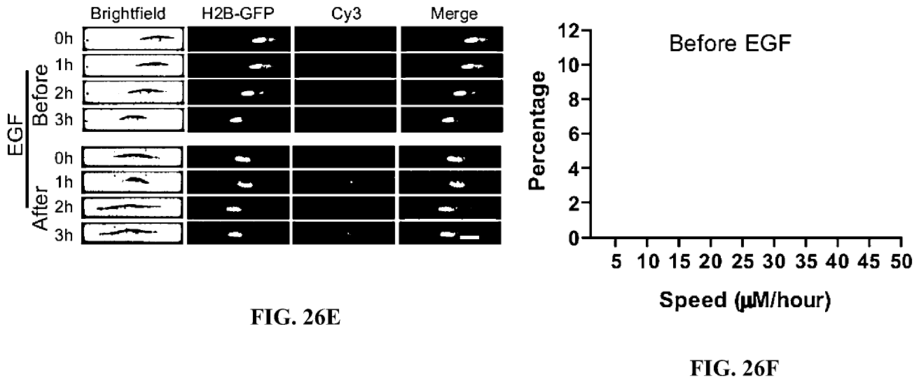

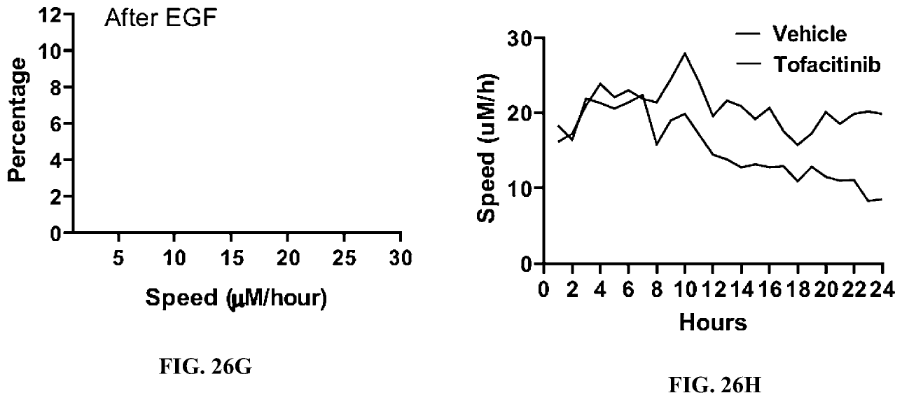

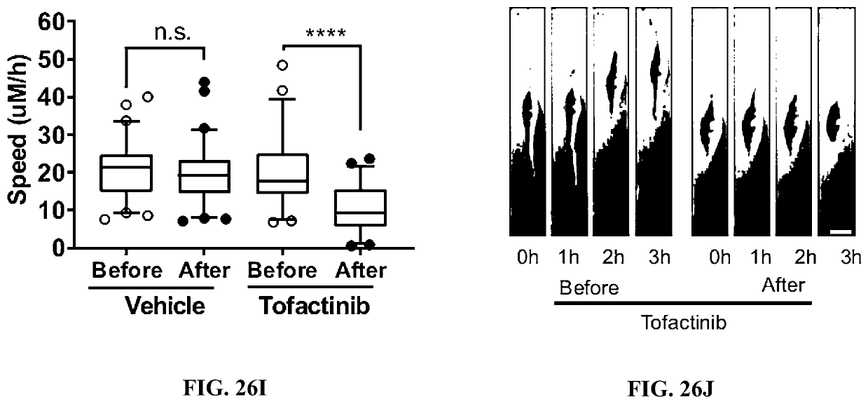

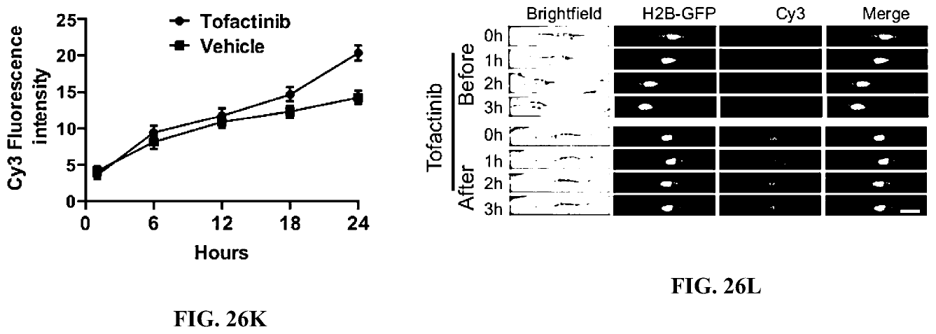

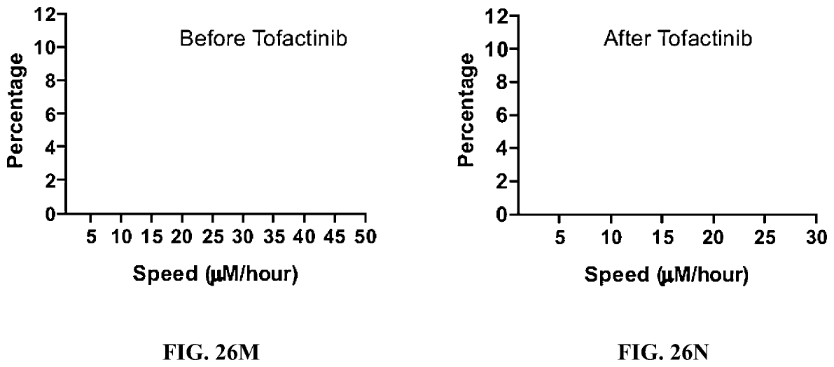

To further investigate the invasive behavior of individual glioma cells, a nanoplate fabricated with topographic patterns of regular parallel ridges that mimic the ECM environment in brain and has been used previously in invasion studies of GBM75 was used. Cells were incubated in nanoplates in the absence or presence of EGF and live cell imaging was conducted over time. It was first investigated whether individual glioma cells can be arrested in their invasion by addition of EGFR ligand. Indeed, it was found that exposure of glioma cells to EGF resulted in a significant slowing of migration of GBM12 cells (FIG. 25A-C). Tracking the movement of individual cells after the addition of EGF shows a suppression of cell motility (FIG. 25A-C). Monitoring movement and proliferation simultaneously in the same cell over time demonstrates that addition of EGF suppresses motility and induces proliferation as detected using a Cy3 linked marker (FIG. 25D and FIG. 25E). EGF-mediated proliferation was also confirmed by Ki-67 staining in the nanoplates (FIG. 25F and FIG. 25G). Similar results were obtained with a second PDX line GBM22 (FIG. 26A-G). GBMs exhibit a significant heterogeneity and have also been reported to exhibit significant heterogeneity even within the same clone in the response to PDGF in single cell motility analysis on nanoplates with similar topography (Smith, et al. (2016) Migration Phenotype of Brain-Cancer Cells Predicts Patient Outcomes, Cell Rep 15: 2616-2624). Not surprisingly, it was found that there was heterogeneity in migration speed both with and without EGF within the same PDX line (FIG. 25H, FIG. 25I, FIG. 26F, and FIG. 26G). Without wishing to be bound by theory, these data clearly demonstrate that EGF suppresses motility and induces proliferation in single cell analysis and also show that the expected heterogeneity in response to EGF. The effect of tofacitinib was also confirmed in the single cell analysis. The effect of tofacitinib is similar to EGF resulting in decreased invasion and increased proliferation (FIG. 25J-N). In addition, the expected heterogeneity was also found in suppression of motility in response to tofacitinib (FIG. 25O and FIG. 25P). The results with tofacitinib were confirmed in a second PDX line (FIG. 26H-N).

Referring to FIGS. 25A-P, EGF and tofacitinib result in reduced migration and enhanced proliferation in single cell analysis. Specifically, migration speed time-lapse in GBM12 before and after addition of EGF (50 ng/ml) is shown in FIG. 25A. Analysis of individual cellular movement was performed as described elsewhere herein. EGF (50 ng/ml) or vehicle was added 6 hours after the start of time-lapse imaging (n=63 cells). Vehicle was used as a negative control (n=67 cells). Single cell migration speed was calculated using Manual tracking Plugin in ImageJ. FIG. 25B shows the quantification of migration velocity of GBM12 before and after addition of EGF (n=63) or vehicle (n=67). Cell migration velocity was calculated over a period of 6 hours. Representative static time-lapse images of the same cell migration before and after addition of EGF are shown in FIG. 25C. FIG. 25D shows the quantification of Cy3 intensity in cells before and after addition of EGF (50 ng/ml) or vehicle, Cy3 intensity was evaluated every 6 hours up to 24 hours. Analysis of individual cell proliferation was performed as described elsewhere herein. EGF (50 ng/ml) was added 6 hours after the start of time-lapse imaging (n=56). Vehicle was used as a negative control (n=64). Representative static time-lapse Cy3 and H2B-GFP staining images of the same cell before and after addition of EGF are shown in FIG. 25E. FIG. 25F shows immunofluorescence of Ki67 in GBM12 treated with or without EGF (50 ng/ml). FIG. 25G shows the quantification of Ki67 staining. Each point represents the percentage of Ki positive cells in total 100 cells. The percentage of cell migration speed before and after addition of EGF (n=63) is shown in FIG. 25H and FIG. 25I. FIG. 25J shows migration speed time-lapse in GBM12 before and after addition of 1 μM of tofacitinib (Vehicle group, n=81, tofacitinib group, n=89). FIG. 25K shows the quantification of migration velocity of GBM12 before and after addition of tofacitinib (1 μM). Cell migration velocity was calculated over a period of 6 hours (n=81). Vehicle was used as a negative control (n=89). Representative static time-lapse images of the same cell migration before and after addition of tofacitinib are shown in FIG. 25L. FIG. 25M shows the quantification of Cy3 intensity cells before and after addition of tofacitinib (1 μM) (vehicle group, n=55, tofacitinib, n=63). Representative static time-lapse Cy3 and H2B-GFP staining images of the same cell before and after addition of tofacitinib are shown in FIG. 25N. The percentage of cell migration speed before and after addition of tofacitinib (1 μM) (n=89 cells) is shown in FIG. 25O and FIG. 25P. Scale bar=10 μM. Data are represented as media ±5-95% interquartile range (FIG. 25B and FIG. 25K) or mean±SEM (FIG. 25D and FIG. 25M). ***P<0.001, ***P<0.0001, n.s. not significant, Mann-Whitney test was used for pair comparison. Two-way ANOVA test was used to analyze Cy3 intensity data in FIG. 25D and FIG. 25M. The results are representative of two independently repeated experiments.

Referring to FIGS. 26A-N, EGF and tofacitinib result in reduced cell migration velocity and enhanced cell proliferation. Specifically, migration speed time-lapse microscopy in GBM22 before and after addition of EGF (50 ng/ml) is shown in FIG. 26A. Single-cell migration analysis was performed as described for FIG. 25A above (Vehicle group, n=57, EGF group, n=74). FIG. 26B shows the quantification of migration velocity of GBM22 before and after addition of EGF (n=74). Cell migration velocity was calculated over a period of 6 hours. Vehicle was used as a negative control (n=57). Representative static time-lapse images of the same cell migration before and after addition of EGF are shown in FIG. 26C. FIG. 26D shows the quantification of Cy3 intensity in GBM22 before and after addition of EGF (50 ng/ml), Cy3 intensity was evaluated every 6 hours up to 24 hours. Analysis of individual cell proliferation was performed as described elsewhere herein. EGF (50 ng/ml) was added 6 hours later (n=74). Vehicle was used as a negative control (n=57). Representative static time-lapse Cy3 and H2B-GFP staining images of the same cell before and after addition of EGF are shown in FIG. 26E. The percentage of cell migration speed before and after addition of EGF (n=74 cells) is shown in FIG. 26F and FIG. 26G. FIG. 26H shows the migration speed time-lapse in GBM22 before and after addition of tofacitinib (1 μM). Single-cell migration analysis was performed as described for FIG. 25J above (Vehicle group, n=61, tofacitinib group, n=53). FIG. 26I shows the quantification of migration velocity of GBM22 before and after addition of tofacitinib (1 μM) (n=53). Vehicle was used as a negative control (n=61). Cell migration velocity was calculated over a period of 6 hours. Representative static time-lapse images of the same cell migration before and after addition of tofacitinib are shown in FIG. 26J. FIG. 26K shows the quantification of Cy3 intensity in GBM22 before and after addition of tofacitinib (1 μM) (Vehicle group, n=50, tofacitinib group, n=54). Representative static time-lapse Cy3 and H2B-GFP staining images of the same cell before and after addition of tofacitinib are shown in FIG. 26L. The percentage of cell migration speed before and after addition of tofacitinib (1 μM) (n=53) is shown in FIG. 26M and FIG. 26N. Scale bar=10 μM. Data are represented as media ±5-95% interquartile range (FIG. 26B and FIG. 26I) or mean±SEM (FIG. 26D and FIG. 26K). ***P<0.001, ***P<0.0001, n.s. not significant, Mann-Whitney test was used for pair comparison. Two-way ANOVA test was used to analyze Cy3 intensity data in FIG. 26D and FIG. 26M. The results are representative from two independently repeated experiments.

10. EGFR Ligands and BIN3 in GBM

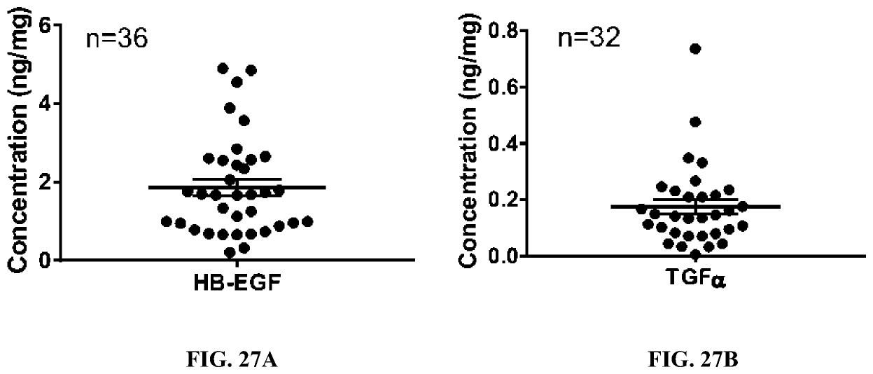

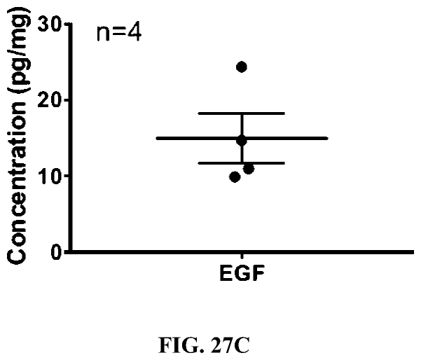

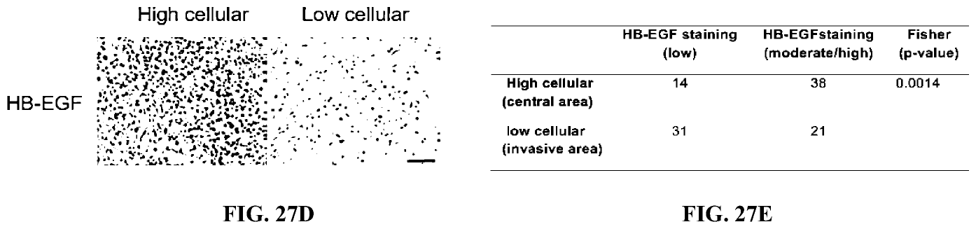

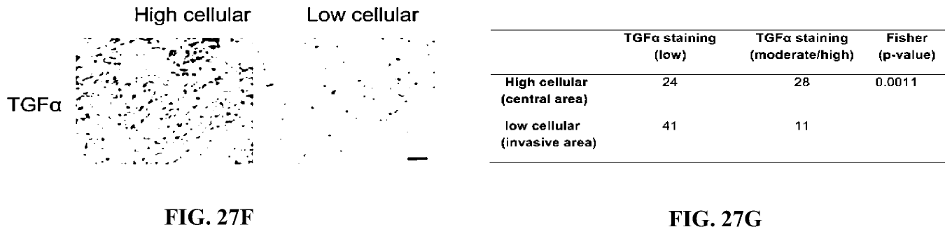

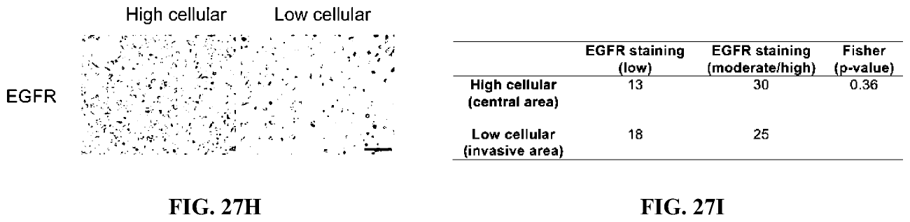

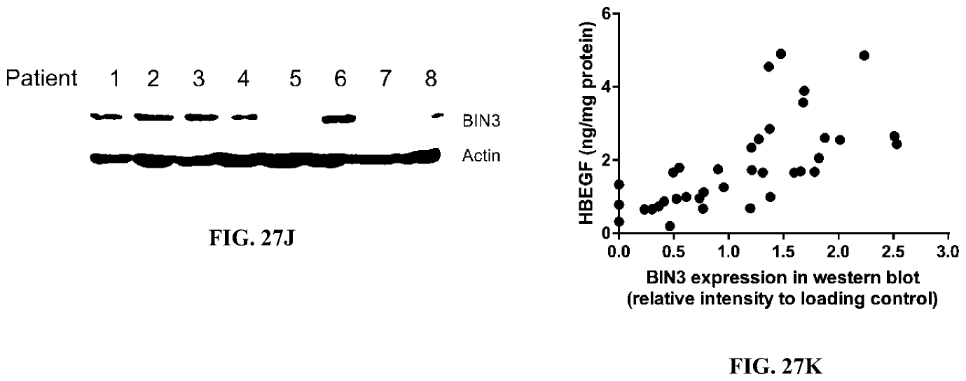

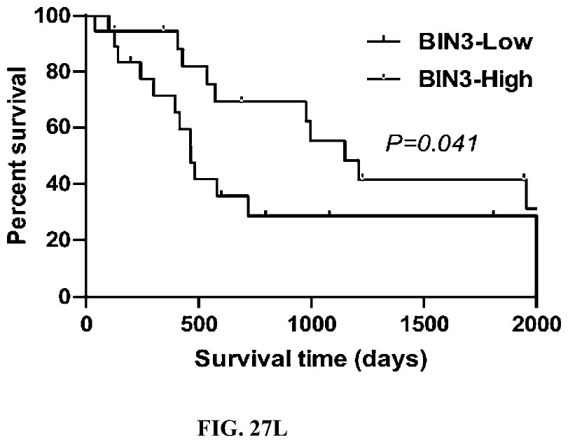

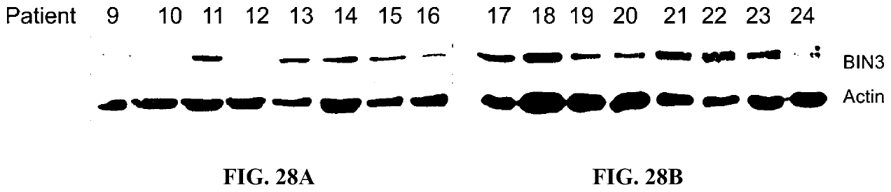

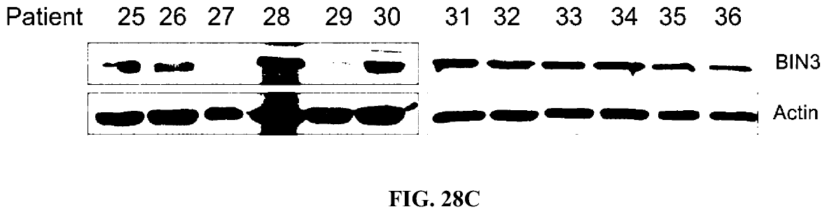

To validate these experimental findings in human GBM, three EGFR ligands were examined, TGFα, HBEGF, and EGF, in 30 resected GBM samples using ELISA. Expression of TGFα and HBEGF was detected, but EGF was very low or undetectable in most samples (FIG. 27A-C). It was also found that HBEGF is much more abundant compared to TGFα in most GBMs. Importantly, there is a large variation in ligand expression among different tumors, demonstrating that some GBMs are EGFR ligand-rich while others are ligand poor. Additionally, a greater expression of EGFR ligands was found in the central part of the tumor compared to the leading edge (FIG. 27D-G). However, the expression level of the EGFR does not vary and is similar in the center and the leading edge (FIG. 27H and FIG. 27I). The expression of BIN3 was also examined, and it was found that while BIN3 is expressed in most GBMs, the expression level varies, and BIN3 expression is undetectable in 10% of cases, consistent with a putative tumor suppressor role (FIG. 27J and FIG. 28A-C). Since HBEGF is the major ligand detected in the cohort of GBMs, whether HBEGF correlates with the expression of BIN3 was examined, and it was found that there is a significant correlation between level of HBEGF and BIN3 (FIG. 27K). Furthermore, a higher level of BIN3 confers an improved prognosis (FIG. 27L). Without wishing to be bound by theory, these data support the model disclosed herein, suggesting that invasion is driven by constitutive EGFR signaling while ligand induced EGFR activation suppresses invasion via upregulation of BIN3. Also, the correlation between HBEGF and BIN3 levels suggest that ligand-induced EGFR signaling is the major regulator of BIN3 level in GBM. The significant variation in EGFR ligand expression among GBMs suggests that it may be possible to stratify patients who are more likely to benefit from tofacitinib.

Referring to FIG. 27A-L, EGFR ligands and BIN3 expression in human glioblastoma is shown. FIG. 27A shows ELISA for HB-EGF in human glioblastoma lysates. FIG. 27B shows ELISA for TGFα in human glioblastoma lysates. FIG. 27C shows ELISA for EGF in human glioblastoma lysates. Representative immunohistochemical staining of HB-EGF in human glioblastoma is shown in FIG. 27D. A summary of HB-EGF staining in high and low cellular areas across the tissue sections from 24 samples is shown in FIG. 27E. Score 0 and 1 are defined as low, 2 and 3 are defined as moderate/high. Two to three random fields from high and low cellular areas per section were picked for evaluation. Staining intensity in the two areas were compared using Fisher's exact test. FIG. 27F shows representative immunohistochemical staining of TGFα in high cellular (central) and low cellular (invasive) areas of human glioblastoma. A summary of TGFα staining of the tissue sections from 24 samples is shown in FIG. 27G. FIG. 27H shows representative immunohistochemical staining of EGFR in high cellular (central) and low cellular (invasive) areas of human glioblastoma. A summary of EGFR staining of the tissue sections from 20 samples is shown in FIG. 27I. FIG. 27J shows a Western blot of BIN3 expression in human glioblastoma extracts. Actin served as a loading control. FIG. 27K shows a scatter plot of HB-EGF and BIN3 expression in human glioblastoma lysates (n=36). Y-axis represents HB-EGF concentration of human globlastoma extracts. X-axis represents the values of relative intensity obtained based on densitometry measurements of BIN3 and Actin control levels on the western blot. Kaplan-Meier curves of survival rates for high and low levels of BIN3 assessed by Western blot are shown in FIG. 27L. Scale bars: 50 μM. Data are represented as mean±SEM from three independent experiments.

Referring to FIG. 28A-C, Western blots of BIN3 expression in resected human glioblastoma samples are shown. Lysates were prepared from frozen tumor tissue. Actin served as a loading control.

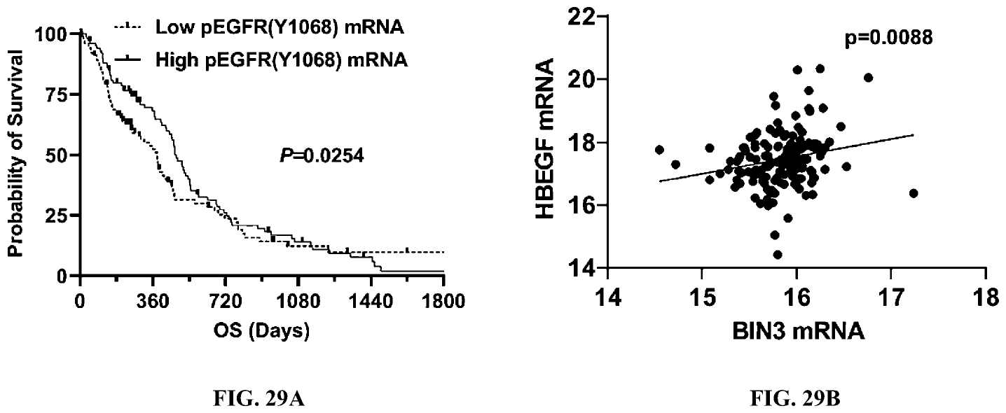

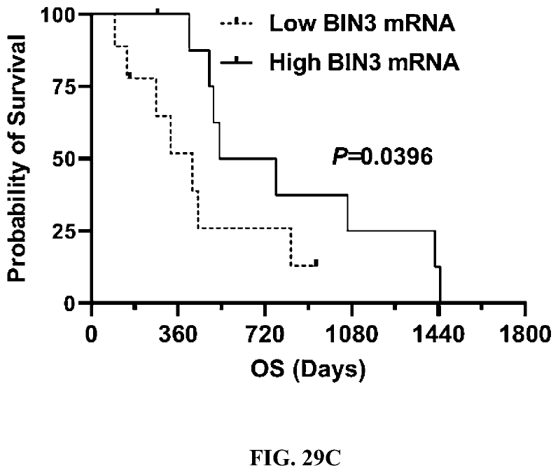

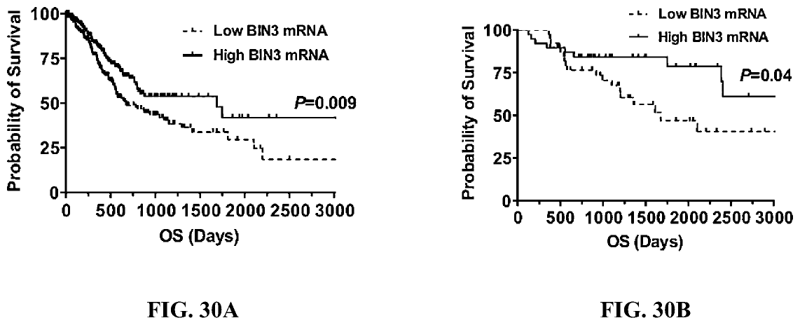

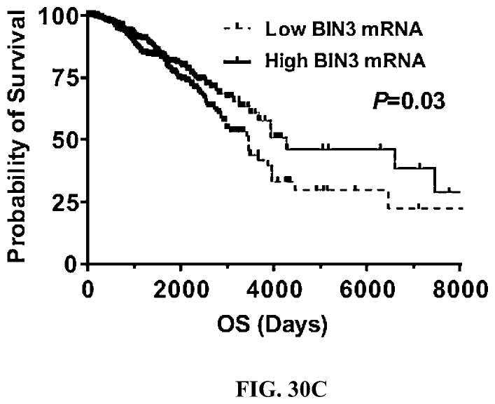

An examination of public databases revealed a number of interesting finding that support these data. Firstly, and importantly, phosphorylation of the EGFR, which correlates with ligand-mediated activation of EGFR, correlated with an improved prognosis (FIG. 29A). Additionally, the level of HB-EGF, which was found to be the major ligand for GBMs in this analysis correlates with BIN3 level (FIG. 29B). BIN3 maps to chromosome 8p21.3 a tumor suppressor region that is often deleted in non-Hodgkin's lymphoma and other epithelial cancers (Birnbaum, et al. (2003) Chromosome arm 8p and cancer: a fragile hypothesis, The lancet oncology 4: 639-642; Rubio-Moscardo, et al. (2005) Characterization of 8p21.3 chromosomal deletions in B-cell lymphoma: TRAIL-R1 and TRAIL-R2 as candidate dosage-dependent tumor suppressor genes, Blood 106: 3214-3222; Chang, et al. (2007) Integration of somatic deletion analysis of prostate cancers and germline linkage analysis of prostate cancer families reveals two small consensus regions for prostate cancer genes at 8p, Cancer Res 67: 4098-4103; Ye, et al. (2007) Genomic assessments of the frequent loss of heterozygosity region on 8p21.3-p22 in head and neck squamous cell carcinoma, Cancer Genet Cytogenet 176: 100-106). BIN3 deletion in mice results in increased susceptibility to lymphoma (Ramalingam, et al. (2008) Bin3 deletion causes cataracts and increased susceptibility to lymphoma during aging, Cancer Res 68: 1683-1690). Another BAR family member BIN1, functions as a tumor suppressor gene in multiple cancer types (Prendergast, et al. (2009) BAR the door: cancer suppression by amphiphysin-like genes, Biochim Biophys Acta 1795: 25-36). Consistent with a putative role of BAR domain porteins as tumor suppressors (Birnbaum, et al. (2003) Chromosome arm 8p and cancer: a fragile hypothesis, The lancet oncology 4: 639-642), an analysis of the TCGA data revealed a significant correlation for BIN3 as a potential tumor suppressor in three cancers (FIG. 30A-C). While, BIN3 does not appear to confer an improved prognosis in GBMs overall in the TCGA data, it did confer an improved prognosis in high EGFR overexpressing GBMs. Thus, when stratified by the level of EGFR expression, BIN3 conferred an improved prognosis in the top 50% of EGFR expressers in classical GBM (FIG. 29C). Without wishing to be bound by theory, these data support a model suggesting that invasion is driven by constitutive EGFR signaling while ligand induced EGFR activation suppresses invasion via upregulation of BIN3 and confers a better prognosis. Also, the correlation between HB-EGF and BIN3 levels suggest that ligand-induced EGFR signaling is the major regulator of BIN3 level in GBM. The significant variation in EGFR ligand expression among GBMs suggests that it may be possible to stratify patients who are more likely to benefit from tofactinib.

Referring to FIG. 29A-C, a survival and correlation analysis according to BIN3, HB-EGF, and EGFR expression is shown. Specifically, FIG. 29A shows an overall survival analysis according to pEGFR expression in patients with GBM. The 204 primary TCGA-GBM patients were divided into high-50% and low-50% groups by pEGFR (Y1068) expression level and the effect on survival was examined (N=204). The Pearson correlation coefficient between BIN3 and HBEGF mRNA was analyzed from 153 TCGA-GBM patients (FIG. 29B). FIG. 29C shows the overall survival analysis according to BIN3 mRNA levels in patients with classical GBMs with amplified EGFR. Among the primary TCGA-GBM patients, 18 classical GBM patients with EGFR top-50% highly amplification (log 2 copy number >3.24) were divided into high-50% and low-50% groups by BIN3 mRNA. The P-value was obtained by Gehan's test of the Kaplan-Meier curve. *: p<0.05.

Referring to FIG. 30A-C, an overall survival analysis according to BIN3 expression in three cancer types is shown. Specifically, FIG. 30A shows an overall survival analysis according to BIN3 mRNA levels in patients with gastric cancer (N=381). FIG. 30B shows an overall survival analysis according to BIN3 mRNA levels in patients with adrenocortical carcinoma (N=79). FIG. 30C shows an overall survival analysis according to BIN3 mRNA levels in patients with breast cancer (N=1195). In all panels patients were divided into high-50% and low-50% groups by BIN3 mRNA levels. The P-value was obtained by log rank test of the Kaplan-Meier curve.

11. Discussion

GBM is a devastating disease primarily because of its highly invasive nature. In this study, a pathway that actively suppresses GBM invasion was identified. This pathway is triggered by ligand-mediated activation of the EGFR that leads to upregulation of the N-BAR domain cytoskeletal protein BIN3. While a number of pathways have been identified that drive invasion in GBM, little is known about whether there are pathways that actively suppress invasion. It is proposed that the EGFR-BIN3 axis plays a key role in suppressing invasion in GBM, and that this pathway could be targeted in a novel therapeutic approach to activate a suppression of invasion. These studies indicate that the mode of EGFR activation is a switch that can promote invasion or proliferation in GBM depending on the presence of EGFR ligand. Since the EGFR is expressed in the majority of GBMs, this EGFR switch may be the key mechanism of spatiotemporal regulation of proliferation and invasion in these tumors. In the absence of ligand, the constitutively active EGFR drives invasion, an increase in tumor size, and a worse prognosis. When EGFR ligand is added, invasion is suppressed and glioma cells proliferate resulting in small tumors that are intensely proliferating but are noninvasive, unable to expand, and have a better prognosis. Thus, although both unrestrained proliferation and invasion are hallmarks of cancer, these data indicate that in GBMs invasion plays a more important role in the regulation of tumor size and prognosis. These data also provide support for the spatiotemporal regulation of proliferation and invasion, and it is proposed that an EGFR switch is a major contributor to this “grow or go” decision. These data are also consistent with a previous study reporting that EGFR signaling is bimodal and that constitutive and ligand-activated EGFR signaling trigger distinct signaling pathways (Chakraborty et al. (2014) Nat. Commun. 5: 5811; Guo et al. (2015) Cancer Res. 75: 3436-3441).

The mechanisms of GBM invasion have been intensely studied and signaling pathways that promote and execute GBM invasion have been identified (Armento et al. (2017) Molecular Mechanisms of Glioma Cell Motility, In Glioblastoma (De Vleeschouwer, S., Ed.), Brisbane (AU)). For example, the RhoGTPase pathway has a critical role in promoting invasiveness. However, it is unknown whether there are pathways that actively suppress invasion. Such a pathway would be of considerable interest, particularly if it could be therapeutically activated. It is proposed that the EGFR-BIN3 signaling pathway identified in this study is a major suppressor of invasiveness. Thus, ligand-induced EGFR activation results in upregulation of BIN3, which in turn suppresses invasion. This is demonstrated in invasion assays and in multiple animal experiments showing that ligand-activated EGFR results in a suppression of invasion, and this suppression is rescued if BIN3 is silenced. Conversely, BIN3 overexpression mimics the effects of ligand-induced EGFR activation in an orthotopic model resulting in small noninvasive tumors with a better prognosis. BIN3 is a member of the Bin-Ampiphysin-Rvs (BAR) domain family of proteins that regulate membrane and actin dynamics, and these data indicate that it is unregulated by ligand-dependent EGFR activation and plays a central role in suppressing invasion both in response to EGFR activation and also upon tofacitinib exposure. Ligand-induced EGFR activation leads to association of BIN3 with DOCK7. DOCK family members have a RhoGEF domain and function as GEFs for the Rho GTPase family. It is proposed that the BIN3-DOCK7 association inhibits the function of DOCK7 by demonstrating a role for DOCK7 in promoting invasion. Also, DOCK7 activity is suppressed by EGF and DOCK7 is required for the EGF medicated downregulation of Rho-GTPase activity. Thus, mechanistically, ligand-induced EGFR activation leads to decreased invasiveness by a BIN3 mediated inhibition of a DOCK7-RhoGTPase pathway. Here, it is demonstrated that ligand-mediated EGFR upregulation leads to induction of the transcription factor EGR1 which, in turn, drives increased transcription of BIN3.

Previous studies using primarily established glioblastoma cell lines have reported that EGF results in increased invasiveness of GBM tumor cells. It was found that while this is true for established GBM cell lines that have lost the EGFR amplification during repeated culture, in the more clinically relevant PDX samples, EGF consistently suppresses invasion. Multiple methods were used to demonstrate the effect of EGF in suppressing invasion. These include ex vivo methods such as transwell invasion assays and wound healing experiments. In addition, single cell analysis was used on surfaces designed to mimic GBM extracellular matrix, and it was again found that ligand-mediated EGFR expression suppresses invasion and promotes proliferation. The single cell analysis also indicate the when cells are stimulated with EGF, they stop invading and start to proliferate, indicating a dissociation between invasion and proliferation induced by the EGFR switch. In vivo, intravital microscopy was used to demonstrate that EGF suppresses invasion. In addition, immunohistochemical studies of tumors in mouse brain using H&E staining, mouse neurofilament staining, and human nuclear markers all unequivocally demonstrate that ligand-induced EGFR activation suppresses invasion. Although the emphasis of this study is on the EGFR-BIN3 pathway that suppresses invasion, a mechanism used by constitutive EGFR signaling to drive invasion has also been identified. Thus, it was found that while constitutive EGFR signaling does not alter BIN3 levels, it does activate a Nanog-EMP1 pathway that drives invasion. Evidence that constitutive EGFR signaling activates Nanog resulting in transcription of EMP1 is shown, and also evidence that loss of either Nanog or EMP1 blocks the ability of constitutive EGFR signaling to drive invasion.

It has been proposed that tumor invasion and proliferation are spatiotemporally distinct and may be mutually exclusive, the “go or grow hypothesis” (Newman, et al. (2017) Interleukin-13 receptor alpha 2 cooperates with EGFRvIII signaling to promote glioblastoma multiforme, Nat Commun 8: 1913; Xie, et al. (2014) Targeting adaptive glioblastoma: an overview of proliferation and invasion, Neuro Oncol 16: 1575-1584; Venere, et al. (2015) The mitotic kinesin KIF11 is a driver of invasion, proliferation, and self-renewal in glioblastoma, Sci Transl Med 7: 304ra143; Hatzikirou, et al. (2012) ‘Go or grow’: the key to the emergence of invasion in tumour progression?, Math Med Biol 29: 49-65; Horing, et al. (2012) The “go or grow” potential of gliomas is linked to the neuropeptide processing enzyme carboxypeptidase E and mediated by metabolic stress, Acta neuropathologica 124: 83-97; Dhruv, et al. (2013) Reciprocal activation of transcription factors underlies the dichotomy between proliferation and invasion of glioma cells, PLoS One 8: e72134; Gao, et al. (2005) Proliferation and invasion: plasticity in tumor cells, Proc Natl Acad Sci USA 102: 10528-10533; Matus, et al. (2015) Invasive Cell Fate Requires G1 Cell-Cycle Arrest and Histone Deacetylase-Mediated Changes in Gene Expression, Dev Cell 35: 162-174; Ewald, A. J. (2015) An Arresting Story about Basement Membrane Invasion, Dev Cell 35: 143-144). However, the molecular regulators of go or grow transitions have remained elusive (Ewald, A. J. (2015) An Arresting Story about Basement Membrane Invasion, Dev Cell 35: 143-144). A number of environmental and intracellular signaling pathways have been implicated in the invasion vs. proliferation decision, including chemoattractants, cytoskeletal dynamics, cell volume, and the extracellular composition (Qin, et al. (2017) Neural Precursor-Derived Pleiotrophin Mediates Subventricular Zone Invasion by Glioma, Cell 170: 845-859 e819; Hatzikirou, et al. (2012) ‘Go or grow’: the key to the emergence of invasion in tumour progression?, Math Med Biol 29: 49-65; Giese, et al. (1996) Dichotomy of astrocytoma migration and proliferation, Int J Cancer 67: 275-282). It has been reported that the pentose phosphate pathway (PPP) is used mainly during proliferation, while glycolysis is used as the energy source during invasion (Kathagen-Buhmann, et al. (2016) Glycolysis and the pentose phosphate pathway are differentially associated with the dichotomous regulation of glioblastoma cell migration versus proliferation, Neuro Oncol 18: 1219-1229). Increased c-Myc activity was reported in proliferating cells while increased NF-κB activation was found in invasive glioma cells (Dhruv, et al. (2013) Reciprocal activation of transcription factors underlies the dichotomy between proliferation and invasion of glioma cells, PLoS One 8: e72134). The molecular motor kinesin KIF11 has been reported to play a role in both proliferation and invasion (Venere, et al. (2015) The mitotic kinesin KIF11 is a driver of invasion, proliferation, and self-renewal in glioblastoma, Sci Transl Med 7: 304ra143). There are also studies that have argued against the grow or grow hypothesis. For example, in a study of medulloblastoma cell lines using time-lapse video-microscopy and Ki-67 found that migrating and invading cells continued to proliferate (Corcoran and Del Maestro (2003) Testing the “Go or Grow” hypothesis in human medulloblastoma cell lines in two and three dimensions, Neurosurgery 53: 174-184; discussion 184-175). Data from another study of lung, melanoma, and mesothelioma cell lines also failed to support the go or grow hypothesis (Garay, et al. (2013) Cell migration or cytokinesis and proliferation?—revisiting the “go or grow” hypothesis in cancer cells in vitro, Exp Cell Res 319, 3094-3103). Thus, it appears that the exclusivity of proliferation and proliferation may be cell type or context dependent, and is unlikely to be absolute. However, these data indicate that ligand activated EGFR switches the phenotype from invasion to proliferation with critical biological consequences, and is likely to be a useful tool to dissect the spatiotemporal regulation of invasion and proliferation.

This study of EGFR ligands uncovers a number of interesting findings. Firstly, there appears to be a wide variation in the level of EGFR ligands expressed in GBMs, with some GBMs expressing low levels of ligand compared to others. Secondly, HB-EGF (heparin binding epidermal growth factor) appears to be the major ligand expressed in GBMs, with TGFα also readily detectable at lesser level but low or undetectable levels of EGF. Thirdly, EGFR ligands are expressed at higher levels in the core of the tumor compared to the leading edge, while the expression of EGFR receptor does not exhibit this regional variation. These experimental findings indicate that a high expression of EGFR ligand results in increased proliferation, decreased invasion, smaller tumors, and an improved prognosis. These findings are consistent with a model in which ligand-independent constitutive EGFR signaling drives invasion, increases tumor size, and confers a worse prognosis, while ligand-induced activation of EGFR switches on proliferation and increases BIN3 to turn off invasion, resulting in smaller hyper-proliferating tumors with an improved prognosis. Thus, the local availability of EGFR ligand is likely to play a critical role in the regulating the spatiotemporal regulation of invasion and proliferation in GBMs. Interestingly, it has also been reported that constitutive EGFR signaling confers a worse prognosis in colorectal cancer (Yun, et al. (2018) Ligand-Independent Epidermal Growth Factor Receptor Overexpression Correlates with Poor Prognosis in Colorectal Cancer, Cancer Res Treat 50: 1351-1361). Consistent with these finding, analysis of TCGA/TCPA data reveals that increased phosphorylation of EGFR (indicative of ligand mediated activation) confers an improved prognosis. Furthermore HB-EGF, the most commonly expressed EGFR ligand in GBM, correlates with BIN3 in GBM (FIG. 29B). These studies indicate that increased BIN3 at a protein level leads to a favorable prognosis in GBM. While a favorable effect of BIN3 mRNA levels could not be detected overall in GBMs in the TCGA data, it was found that in a subset of GBMs with high EGFR levels, BIN3 correlates with an improved prognosis.

The invasiveness of GBMs presents a major barrier to treatment and renders complete surgical resection difficult. Thus, a drug that inhibits GBM invasion could be extremely helpful in the treatment of GBMs. Here, tofacitinib, a JAK1/JAK3 inhibitor, is identified as a drug that upregulates BIN3 and blocks GBM invasion in the experimental model. Interestingly, tofacitinib is unlikely to upregulate BIN3 via inhibition of JAK/STAT pathways since ligand activation of the EGFR activates JAK/STATs and also upregulates BIN3. It was found that tofacitinib upregulates multiple transcription factors presumably as an adaptive response to JAK/STAT inhibition. EGR1 is identified as a transcription factor activated by both ligand-mediated EGFR activation and tofacitinib, and demonstrate that it is required for upregulation of BIN3 by both stimuli. Tofacitinib is less effective in ligand-rich tumors with high BIN3 levels. For example, overexpression of EGFR ligand in PDX lines results in BIN3 upregulation, decreased invasion, and improved survival in an orthotopic model. However, treatment of such tumors with tofacitinib does not upregulate BIN3 further and has no further impact on invasion or prognosis, whereas in vector transfected control lines with no overexpression of ligand, tofacitinib results in upregulation of BIN3, a significant suppression of invasion and improved survival. Furthermore, in PDX lines with high endogenous EGFR ligand expression and high BIN3 levels, tofacitinib is ineffective in upregulating BIN3 or inhibiting invasion. siRNA knockdown of EGFR ligand in such PDX lines renders them responsive to tofacitinib, which now upregulates BIN3 and suppresses invasion in vitro and in vivo. It is proposed that these findings suggest a therapeutic opportunity for this devastating disease. Tofacitinib could be a unique and effective treatment for GBM that specifically targets invasion, and may be more helpful in EGFR ligand poor GBMs.

### G. REFERENCES

Altieri, R., Zenga, F., Fontanella, M. M., Cofano, F., Agnoletti, A., Spena, G., Crobeddu, E., Fornaro, R., Ducati, A., and Garbossa, D. (2015) Glioma Surgery: Technological Advances to Achieve a Maximal Safe Resection, Surg Technol Int 27, 297-302.
Armento, A., Ehlers, J., Schotterl, S., and Naumann, U. (2017) Molecular Mechanisms of Glioma Cell Motility, In Glioblastoma (De Vleeschouwer, S., Ed.), Brisbane (AU).
Xie, Q., Mittal, S., and Berens, M. E. (2014) Targeting adaptive glioblastoma: an overview of proliferation and invasion, Neuro Oncol 16, 1575-1584.
Venere, M., Horbinski, C., Crish, J. F., Jin, X., Vasanji, A., Major, J., Burrows, A. C., Chang, C., Prokop, J., Wu, Q., Sims, P. A., Canoll, P., Summers, M. K., Rosenfeld, S. S., and Rich, J. N. (2015) The mitotic kinesin KIF11 is a driver of invasion, proliferation, and self-renewal in glioblastoma, Sci Transl Med 7, 304ra143.
Hatzikirou, H., Basanta, D., Simon, M., Schaller, K., and Deutsch, A. (2012) ‘Go or grow’: the key to the emergence of invasion in tumour progression?, Math Med Biol 29, 49-65.
Horing, E., Harter, P. N., Seznec, J., Schittenhelm, J., Buhring, H. J., Bhattacharyya, S., von Hattingen, E., Zachskorn, C., Mittelbronn, M., and Naumann, U. (2012) The “go or grow” potential of gliomas is linked to the neuropeptide processing enzyme carboxypeptidase E and mediated by metabolic stress, Acta neuropathologica 124, 83-97.
Newman, J. P., Wang, G. Y., Arima, K., Guan, S. P., Waters, M. R., Cavenee, W. K., Pan, E., Aliwarga, E., Chong, S. T., Kok, C. Y. L., Endaya, B. B., Habib, A. A., Horibe, T., Ng, W. H., Ho, I. A. W., Hui, K. M., Kordula, T., and Lam, P. Y. P. (2017) Interleukin-13 receptor alpha 2 cooperates with EGFRvIII signaling to promote glioblastoma multiforme, Nat Commun 8, 1913.
Dhruv, H. D., McDonough Winslow, W. S., Armstrong, B., Tuncali, S., Eschbacher, J., Kislin, K., Loftus, J. C., Tran, N. L., and Berens, M. E. (2013) Reciprocal activation of transcription factors underlies the dichotomy between proliferation and invasion of glioma cells, PLoS One 8, e72134.
Gao, C. F., Xie, Q., Su, Y. L., Koeman, J., Khoo, S. K., Gustafson, M., Knudsen, B. S., Hay, R., Shinomiya, N., and Vande Woude, G. F. (2005) Proliferation and invasion: plasticity in tumor cells, Proc Natl Acad Sci USA 102, 10528-10533.
Giese, A., Loo, M. A., Tran, N., Haskett, D., Coons, S. W., and Berens, M. E. (1996) Dichotomy of astrocytoma migration and proliferation, Int J Cancer 67, 275-282.
Kathagen-Buhmann, A., Schulte, A., Weller, J., Holz, M., Herold-Mende, C., Glass, R., and Lamszus, K. (2016) Glycolysis and the pentose phosphate pathway are differentially associated with the dichotomous regulation of glioblastoma cell migration versus proliferation, Neuro Oncol 18, 1219-1229.
Talasila, K. M., Soentgerath, A., Euskirchen, P., Rosland, G. V., Wang, J., Huszthy, P. C., Prestegarden, L., Skaftnesmo, K. O., Sakariassen, P. O., Eskilsson, E., Stieber, D., Keunen, O., Brekka, N., Moen, I., Nigro, J. M., Vintermyr, O. K., Lund-Johansen, M., Niclou, S., Mork, S. J., Enger, P. O., Bjerkvig, R., and Miletic, H. (2013) EGFR wild-type amplification and activation promote invasion and development of glioblastoma independent of angiogenesis, Acta neuropathologica 125, 683-698.
Roos, A., Dhruv, H. D., Peng, S., Inge, L. J., Tuncali, S., Pineda, M., Millard, N., Mayo, Z., Eschbacher, J. M., Loftus, J. C., Winkles, J. A., and Tran, N. L. (2018) EGFRvIII-Stat5 Signaling Enhances Glioblastoma Cell Migration and Survival, Mol Cancer Res 16, 1185-1195.
Ding, Z., Roos, A., Kloss, J., Dhruv, H., Peng, S., Pirrone, P., Eschbacher, J. M., Tran, N. L., and Loftus, J. C. (2018) A Novel Signaling Complex between TROY and EGFR Mediates Glioblastoma Cell Invasion, Mol Cancer Res 16, 322-332.
Huang, P. H., Xu, A. M., and White, F. M. (2009) Oncogenic EGFR signaling networks in glioma, Sci Signal 2, re6.
Hatanpaa, K. J., Burma S., Zhao D., Habib, A. A. (2010) Epidermal growth factor receptor (EGFR) in glioma: Signal transduction, neuropathology, imaging and radioresistance Neoplasia 12, 675-684.
Habermann, B. (2004) The BAR-domain family of proteins: a case of bending and binding?, EMBO reports 5, 250-255.
de Kreuk, B. J., and Hordijk, P. L. (2012) Control of Rho GTPase function by BAR-domains, Small GTPases 3, 45-52.
Simionescu-Bankston, A., Leoni, G., Wang, Y., Pham, P. P., Ramalingam, A., DuHadaway, J. B., Faundez, V., Nusrat, A., Prendergast, G. C., and Pavlath, G. K. (2013) The N-BAR domain protein, Bin3, regulates Rac1- and Cdc42-dependent processes in myogenesis, Dev Biol 382, 160-171.
Fortin Ensign, S. P., Mathews, I. T., Symons, M. H., Berens, M. E., and Tran, N. L. (2013) Implications of Rho GTPase Signaling in Glioma Cell Invasion and Tumor Progression, Front Oncol 3, 241.
Birnbaum, D., Adelaide, J., Popovici, C., Charafe-Jauffret, E., Mozziconacci, M. J., and Chaffanet, M. (2003) Chromosome arm 8p and cancer: a fragile hypothesis, The lancet oncology 4, 639-642.
Rubio-Moscardo, F., Blesa, D., Mestre, C., Siebert, R., Balasas, T., Benito, A., Rosenwald, A., Climent, J., Martinez, J. I., Schilhabel, M., Karran, E. L., Gesk, S., Esteller, M., deLeeuw, R., Staudt, L. M., Fernandez-Luna, J. L., Pinkel, D., Dyer, M. J., and Martinez-Climent, J. A. (2005) Characterization of 8p21.3 chromosomal deletions in B-cell lymphoma: TRAIL-R1 and TRAIL-R2 as candidate dosage-dependent tumor suppressor genes, Blood 106, 3214-3222.
Chang, B. L., Liu, W., Sun, J., Dimitrov, L., Li, T., Turner, A. R., Zheng, S. L., Isaacs, W. B., and Xu, J. (2007) Integration of somatic deletion analysis of prostate cancers and germline linkage analysis of prostate cancer families reveals two small consensus regions for prostate cancer genes at 8p, Cancer Res 67, 4098-4103.
Ye, H., Pungpravat, N., Huang, B. L., Muzio, L. L., Mariggio, M. A., Chen, Z., Wong, D. T., and Zhou, X. (2007) Genomic assessments of the frequent loss of heterozygosity region on 8p21.3-p22 in head and neck squamous cell carcinoma, Cancer Genet Cytogenet 176, 100-106.
Ramalingam, A., Duhadaway, J. B., Sutanto-Ward, E., Wang, Y., Dinchuk, J., Huang, M., Donover, P. S., Boulden, J., McNally, L. M., Soler, A. P., Muller, A. J., Duncan, M. K., and Prendergast, G. C. (2008) Bin3 deletion causes cataracts and increased susceptibility to lymphoma during aging, Cancer Res 68, 1683-1690.
Prendergast, G. C., Muller, A. J., Ramalingam, A., and Chang, M. Y. (2009) BAR the door: cancer suppression by amphiphysin-like genes, Biochim Biophys Acta 1795, 25-36.
Verhaak, R. G., Hoadley, K. A., Purdom, E., Wang, V., Qi, Y., Wilkerson, M. D., Miller, C. R., Ding, L., Golub, T., Mesirov, J. P., Alexe, G., Lawrence, M., O'Kelly, M., Tamayo, P., Weir, B. A., Gabriel, S., Winckler, W., Gupta, S., Jakkula, L., Feiler, H. S., Hodgson, J. G., James, C. D., Sarkaria, J. N., Brennan, C., Kahn, A., Spellman, P. T., Wilson, R. K., Speed, T. P., Gray, J. W., Meyerson, M., Getz, G., Perou, C. M., and Hayes, D. N. (2010) Integrated genomic analysis identifies clinically relevant subtypes of glioblastoma characterized by abnormalities in PDGFRA, IDH1, EGFR, and NF1, Cancer Cell 17, 98-110.
Frederick, L., Wang, X. Y., Eley, G., and James, C. D. (2000) Diversity and frequency of epidermal growth factor receptor mutations in human glioblastomas, Cancer Res 60, 1383-1387.
Fan, Q. W., Cheng, C. K., Gustafson, W. C., Charron, E., Zipper, P., Wong, R. A., Chen, J., Lau, J., Knobbe-Thomsen, C., Weller, M., Jura, N., Reifenberger, G., Shokat, K. M., and Weiss, W. A. (2013) EGFR Phosphorylates Tumor-Derived EGFRvIII Driving STAT3/5 and Progression in Glioblastoma, Cancer Cell 24, 438-449.
Batra, S. K., Castelino-Prabhu, S., Wikstrand, C. J., Zhu, X., Humphrey, P. A., Friedman, H. S., and Bigner, D. D. (1995) Epidermal growth factor ligand-independent, unregulated, cell-transforming potential of a naturally occurring human mutant EGFRvIII gene, Cell Growth Differ 6, 1251-1259.
Acquaviva, J., Jun, H. J., Lessard, J., Ruiz, R., Zhu, H., Donovan, M., Woolfenden, S., Boskovitz, A., Raval, A., Bronson, R. T., Pfannl, R., Whittaker, C. A., Housman, D. E., and Charest, A. (2011) Chronic activation of wild-type epidermal growth factor receptor and loss of Cdkn2a cause mouse glioblastoma formation, Cancer Res 71, 7198-7206.
Di Fiore, P. P., Pierce, J. H., Fleming, T. P., Hazan, R., Ullrich, A., King, C. R., Schlessinger, J., and Aaronson, S. A. (1987) Overexpression of the human EGF receptor confers an EGF-dependent transformed phenotype to NIH 3T3 cells, Cell 51, 1063-1070.
Velu, T. J., Beguinot, L., Vass, W. C., Willingham, M. C., Merlino, G. T., Pastan, I., and Lowy, D. R. (1987) Epidermal-growth-factor-dependent transformation by a human EGF receptor proto-oncogene, Science 238, 1408-1410.
Nishikawa, R., Ji, X. D., Harmon, R. C., Lazar, C. S., Gill, G. N., Cavenee, W. K., and Huang, H. J. (1994) A mutant epidermal growth factor receptor common in human glioma confers enhanced tumorigenicity, Proc Natl Acad Sci USA 91, 7727-7731.
Chakraborty, S., Li, L., Puliyappadamba, V. T., Guo, G., Hatanpaa, K. J., Mickey, B., Souza, R. F., Vo, P., Herz, J., Chen, M. R., Boothman, D. A., Pandita, T. K., Wang, D. H., Sen, G. C., and Habib, A. A. (2014) Constitutive and ligand-induced EGFR signalling triggers distinct and mutually exclusive downstream signalling networks, Nat Commun 5, 5811.
Wong, A. J., Ruppert, J. M., Bigner, S. H., Grzeschik, C. H., Humphrey, P. A., Bigner, D. S., and Vogelstein, B. (1992) Structural alterations of the epidermal growth factor receptor gene in human gliomas, Proc Natl Acad Sci USA 89, 2965-2969.
Guo, G., Gong, K., Wohlfeld, B., Hatanpaa, K. J., Zhao, D., and Habib, A. A. (2015) Ligand-Independent EGFR Signaling, Cancer Res 75, 3436-3441.
Endres, N. F., Das, R., Smith, A. W., Arkhipov, A., Kovacs, E., Huang, Y., Pelton, J. G., Shan, Y., Shaw, D. E., Wemmer, D. E., Groves, J. T., and Kuriyan, J. (2013) Conformational coupling across the plasma membrane in activation of the EGF receptor, Cell 152, 543-556.
Tan, X., Lambert, P. F., Rapraeger, A. C., and Anderson, R. A. (2016) Stress-Induced EGFR Trafficking: Mechanisms, Functions, and Therapeutic Implications, Trends Cell Biol 26, 352-366.
Carlson, B. L., Pokorny, J. L., Schroeder, M. A., and Sarkaria, J. N. (2011) Establishment, maintenance and in vitro and in vivo applications of primary human glioblastoma multiforme (GBM) xenograft models for translational biology studies and drug discovery, Curr Protoc Pharmacol Chapter 14, Unit 14 16.

Guo, G., Gong, K., Ali, S., Ali, N., Shallwani, S., Hatanpaa, K. J., Pan, E., Mickey, B., Burma, S., Wang, D. H., Kesari, S., Sarkaria, J. N., Zhao, D., and Habib, A. A. (2017) A TNF-JNK-Axl-ERK signaling axis mediates primary resistance to EGFR inhibition in glioblastoma, Nat Neurosci 20, 1074-1084.
Sun, C., and Bernards, R. (2014) Feedback and redundancy in receptor tyrosine kinase signaling: relevance to cancer therapies, Trends in biochemical sciences 39, 465-474.
Misek, S. A., Chen, J., Schroeder, L., Rattanasinchai, C., Sample, A., Sarkaria, J. N., and Gallo, K. A. (2017) EGFR Signals through a DOCK180-MLK3 Axis to Drive Glioblastoma Cell Invasion, Mol Cancer Res 15, 1085-1095.
Jiang, Y., Wang, Y., Wang, T., Hawke, D. H., Zheng, Y., Li, X., Zhou, Q., Majumder, S., Bi, E., Liu, D. X., Huang, S., and Lu, Z. (2014) PKM2 phosphorylates MLC2 and regulates cytokinesis of tumour cells, Nat Commun 5, 5566.
Puliyappadamba, V. T., Chakraborty, S., Chauncey, S. S., Li, L., Hatanpaa, K. J., Mickey, B., Noorani, S., Shu, H. K., Burma, S., Boothman, D. A., and Habib, A. A. (2013) Opposing Effect of EGFRWT on EGFRvIII-Mediated NF-kappaB Activation with RIP1 as a Cell Death Switch, Cell Rep 4, 764-775.
Ramnarain, D. B., Park, S., Lee, D. Y., Hatanpaa, K. J., Scoggin, S. O., Otu, H., Libermann, T. A., Raisanen, J. M., Ashfaq, R., Wong, E. T., Wu, J., Elliott, R., and Habib, A. A. (2006) Differential gene expression analysis reveals generation of an autocrine loop by a mutant epidermal growth factor receptor in glioma cells, Cancer Res 66, 867-874.
Coll, P. M., Rincon, S. A., Izquierdo, R. A., and Perez, P. (2007) Hob3p, the fission yeast ortholog of human BIN3, localizes Cdc42p to the division site and regulates cytokinesis, EMBO J 26, 1865-1877.
Peter, B. J., Kent, H. M., Mills, I. G., Vallis, Y., Butler, P. J., Evans, P. R., and McMahon, H. T. (2004) BAR domains as sensors of membrane curvature: the amphiphysin BAR structure, Science 303, 495-499.
Gadea, G., and Blangy, A. (2014) Dock-family exchange factors in cell migration and disease, Eur J Cell Biol 93, 466-477.
Nakamuta, S., Yang, Y. T., Wang, C. L., Gallo, N. B., Yu, J. R., Tai, Y., and Van Aelst, L. (2017) Dual role for DOCK7 in tangential migration of interneuron precursors in the postnatal forebrain, J Cell Biol 216, 4313-4330.
Murray, D. W., Didier, S., Chan, A., Paulino, V., Van Aelst, L., Ruggieri, R., Tran, N. L., Byrne, A. T., and Symons, M. (2014) Guanine nucleotide exchange factor Dock? mediates HGF-induced glioblastoma cell invasion via Rac activation, Br J Cancer 110, 1307-1315.
Yamamoto, K., Murata, H., Putranto, E. W., Kataoka, K., Motoyama, A., Hibino, T., Inoue, Y., Sakaguchi, M., and Huh, N. H. (2013) DOCK7 is a critical regulator of the RAGE-Cdc42 signaling axis that induces formation of dendritic pseudopodia in human cancer cells, Oncology reports 29, 1073-1079.
Tang, P., Steck, P. A., and Yung, W. K. (1997) The autocrine loop of TGF-alpha/EGFR and brain tumors, J Neurooncol 35, 303-314.
Bachoo, R. M., Maher, E. A., Ligon, K. L., Sharpless, N. E., Chan, S. S., You, M. J., Tang, Y., DeFrances, J., Stover, E., Weissleder, R., Rowitch, D. H., Louis, D. N., and DePinho, R. A. (2002) Epidermal growth factor receptor and Ink4a/Arf: convergent mechanisms governing terminal differentiation and transformation along the neural stem cell to astrocyte axis, Cancer Cell 1, 269-277.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for treating a subject for glioma, the method comprising administering to the subject an effective amount of an agent that modulates bridging integrator 3 (BIN3) signaling, or a pharmaceutically acceptable salt thereof.

2. The method of claim 1, wherein the agent that modulates BIN3 signaling is a BIN3 activator having an EC₅₀ of less than about 100 nM.

3. (canceled)

4. The method of claim 2, wherein the BIN3 activator inhibits janus kinase 3 (JAK3) signaling with an IC₅₀ of less than about 1 nM.

5. (canceled)

6. The method of claim 2, wherein the BIN3 activator inhibits janus kinase 1 (JAK1) signaling with an IC₅₀ of less than about 130 nM.

7. (canceled)

8. The method of claim 2, wherein the BIN3 activator inhibits JAK1 and JAK3 signaling.

9. The method of claim 2, wherein the BIN3 activator inhibits janus kinase 2 (JAK2) signaling with an IC₅₀ of about 20 nM or more.

10. (canceled)

11. The method of claim 1, wherein the agent that modulates BIN3 signaling is tofacitinib.

12. The method of claim 1, further comprising administering to the subject an effective amount of an agent that modulates epidermal growth factor receptor (EGFR) signaling, or a pharmaceutically acceptable salt thereof.

13-18. (canceled)

19. The method of claim 12, wherein the agent that modulates BIN3 signaling is tofacitinib and wherein the agent that modulates EGFR signaling is erlotinib.

20-23. (canceled)

24. The method of claim 1, further comprising administering to the subject an effective amount of an agent associated with the treatment of glioma.

25. (canceled)

26. The method of claim 24, wherein the agent that modulates BIN3 signaling is tofacitinib and wherein the agent associated with the treatment of glioma is temozolomide.

26-29. (canceled)

30. The method of claim 1, further comprising administering to the subject an effective amount of a glucocorticoid.

31-38. (canceled)

39. The method of claim 1, further comprising administering to the subject an effective amount of an EGFR ligand.

40. The method of claim 40, wherein the EGFR ligand is selected from EGF, TGFA, HB-EGF, AR, EREG, BTC, and EPGN.

41. The method of claim 40, wherein the EGFR ligand is EGF.

42-47. (canceled)

48. The method of claim 1, wherein the glioma is a glioblastoma.

49-51. (canceled)

52. A method for treating a subject for glioma, the method comprising administering to the subject an effective amount of an agent that modulates JAK3 signaling, or a pharmaceutically acceptable salt thereof.

53. The method of claim 52, wherein the agent that modulates JAK3 signaling is a JAK3 inhibitor selected from AZD1480, tofacitinib, tofacitinib citrate, WHI-P154, ZM 39923 HCl, PF-06651600, JANEX-1, FM-381, decernotinib, WHI-P258, and WHI-P97.

54-108. (canceled)

109. A method for treating a glioma in a subject in need thereof, the method comprising administering to the subject an agent that increases EGFR ligand.

110-111. (canceled)

112. The method of claim 109, wherein the agent that increases EGFR ligand is an EGFR ligand selected from EGF, TGFA, HB-EGF, AR, EREG, BTC, and EPGN.

113-122. (canceled)
